(19) United States
(12) Patent Application Publication
(10) Pub. No.: US 2007/0196395 A1
(43) Pub. Date: Aug. 23, 2007

(54) Immunomodulatory compounds that target and inhibit the py'binding site of tyrosene kinase p56 lck sh2 domain

(76) Inventors: Alexander Mackerell, Baltimore, MD (US); Jun Hayashi, Ellicott City, MD (US); Ashish Nagarsekar, Gaithersburg, MD (US); Niu Huang, San Francisco, CA (US); Alba Macias, Cambridge (GB)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/582,640

(22) PCT Filed: Dec. 12, 2003
(86) PCT No.: PCT/US03/39501

§ 371 Date: Apr. 20, 2007

(51) Int. Cl. A61K 31/33

(52) U.S. Cl. 424/277.1; 424/184.1

(57) ABSTRACT

Small molecular-weight non-peptidic compounds block Lck SH2 domain-dependent interactions. The inhibitors omit phosphotyrosine (pY) or related moieties.

The protein p56 Lck (Lymphoid T cell tyrosine kinase) is a member of the Src family of tyrosine kinases and is predominantly expressed in T lymphocytes and natural killer cells where it plays a critical role in T-cell-mediated immune responses.^{1,2} p56 Lck is responsible for the phosphorylation of conserved tyrosine residues of CD3 chains, called immunoreceptor tyrosine-based activation motifs (ITAMs), the first step required for T cell activation signaling cascades.^{3,4} Failure of the p56 Lck SH2 domain to bind to ITAMs of CD3 will hamper the T cell receptor (TCR) proximal activation process and suppress the downstream T cell activation signaling cascades.^{3,5} Lck participates in phosphotyrosine (pY)-dependent protein-protein interactions through its modular binding units, called src Homology-2 (SH2) domains.⁶ Accordingly, ligands that are able to block Lck SH2 domain-dependent protein-protein interactions will ultimately find therapeutic utility as immunosuppressants and in the treatment of T cell leukemias, lymphomas and autoimmune diseases such as rheumatoid arthritis.^{2,7}

A phosphopeptide library screen has identified a preferred pY containing peptide binding sequence Ac-pY-E-E-I for the Lck SH2 domain.⁸ This tetrapeptide is an attractive lead structure for the rational design of agents to compete with the SH2 domain's natural ligands. Unfortunately, the tetrapeptide Ac-pY-E-E-I has several undesirable features that hinder its ability to elicit a response in cell-based assays of T-cell activation. First, the phosphate group, an essential element for peptide binding to the SH2 domain, is metabolically unstable to phosphatases present in cells and, secondly, the five negative charges at physiological pH and the high peptidic character may limit its ability to reach efficacious concentrations inside the cell. Due to the conservation of the pY binding site, a pY or similar functional group is strictly required to maintain the peptide binding.¹ Attempts to design SH2 inhibitors with high receptor binding affinity, chemical stability and minimally charged phosphate group replacements have met with limited success.^{9,11} Accordingly, novel approaches towards the identification of p56 Lck SH2 domain inhibitors that avoid the problems associated with the strategies applied to date are required.

High resolution X-ray structures of the Lck SH2 domain complexed with the pY-E-E-I type peptide have provided a 3D molecular map revealing that the pY and Ile residues of the peptide are bound to two well-defined cavities, referred to as the pY and pY+3 binding sites, where the interaction resembles a two-pronged plug engaging a two-holed socket.¹ This binding mode is consistent with experimental observation that SH2 affinity is strongly dependent on the pY and Ile side chains.¹² Moreover, site mutations of amino acid residues in pY+3 binding site switched the binding specificity ¹³⁻¹⁵, which has led to the proposal that the pY+3 binding pocket is also important for specific binding.¹² Thus, the pY+3 site represents a novel target site for the application of rational drug design approaches to identify non-peptidic, specific inhibitors of the p56 Lck SH2 domain.

By using virtual screening methods one can provide an indication as to whether an inventive compound has the proper “fit” to, and is complementary to, a region of the protein which is important for specificity of binding, e.g., a p56^{lck} SH2 domain, as opposed to, e.g., Hck, Fyn, Src, Shc or ZAP-70 SH2 domains. In particular, such methods can indicate whether a compound is complementary to the pY+3 binding site of p56^{lck}. The terms “specific binding” or “specificity of binding” as used herein mean that an inventive compound interacts with, or forms or undergoes a physical association with, a particular SH2 domain (e.g., a p56^{lck} SH2 domain) with a higher affinity, e.g., a higher degree of selectivity, than for other protein moieties (e.g., SH2 domains of other protein kinases).

Virtual screening techniques followed by experimental assays have been used to identify small molecular-weight (MW) non-peptidic compounds targeting the pY+3 binding site that are potent inhibitors of the Lck SH2 domain.

In one embodiment, the invention relates to a method of achieving an immunomodulatory effect in a patient in need thereof, comprising administering an effective amount of one or more of the compounds of one or more of the formulae I to XVII, preferably of formulae I to IX, or a salt thereof.

In another embodiment, the invention relates to a method for achieving an antineoplastic effect in a patient in need thereof, comprising administering an effective amount of a compound of formulae I to XVII, preferably of formulae I to IX, or a salt thereof.

In another embodiment, the invention relates to a method of modulating the binding of a p56^{lck} molecule via an SH2 domain thereof to a corresponding cellular binding protein, and/or modulating the activity of a p56^{lck} molecule via binding to an SH2 domain thereof, comprising binding to an SH2 domain of said p56^{lck} molecule to a compound of formulae I to XVII, preferably of formulae I to IX, or a salt thereof.

In another embodiment, the invention relates to a method of inhibiting hyperproliferative cell growth in a patient in need thereof, comprising administering an effective amount of a compound of formulae I to XVII, preferably of formulae I to IX, or a salt thereof.

In the following formulae, compounds that are not possible are not intended to be included in the definition of the invention. For example, in case of a substituted group ═O on a heterocyclic ring that is defined to be saturated or partially or fully unsaturated, it is readily understood, without stating it each time, that such heterocyclic ring can not be fully unsaturated for such an embodiment, or that with an ═O group present the ring will have a degree of “full” unsaturation only to the extend chemically permitted. Additionally, when ring structures are identified as fully unsaturated, an aromatic ring structure is meant, when identified as saturated, a fully substituted ring structure is meant, and when identified as partially unsaturated, it is between being fully unsaturated and saturated.

Compounds of formula I have the following formula 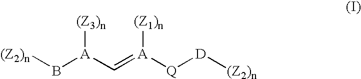
wherein,
B is a phenyl ring,
D is a phenyl ring or a 5-membered saturated or partially or fully unsaturated heterocyclic ring containing 1, 2 or 3, preferably, 1 or 2, heteroatoms selected from O, S, and N,
A is, in each case independently of each other, a 5-membered saturated or partially or fully unsaturated heterocyclic ring containing 1, 2 or 3, preferably, 1 or 2, heteroatoms selected from O, S, and N,
Q is a bond or an alkylene or alkenylene group containing 1-5 carbon atoms, which is optionally substituted with ═O, and in which optionally a carbon atom is replaced with an N atom, and is preferably a bond, ═N— or —C—C(O)—N—,
Z₁ is, in each case independently, —NH₂, ═O, ═NH, or ═N-phenyl, -phenyl, or alkyl containing 1 to 5 carbon atoms, and is preferably —CH₃ when alkyl,
Z₂ is, in each case independently, —OH, halogen, preferably, Cl, F, or Br when halogen, alkyl containing 1-5 carbon atoms, which is optionally substituted with halogen, preferably up to three times with F when substituted with halogen, and/or substituted with ═O and/or —OH, preferably forming a carboxylic acid group when substituted, and in which one C atom is optionally replaced with and O atom, and is preferably, —CH₃ or —COOH,
Z₃ is, in each case independently, alkyl containing 1-5 carbon atoms, preferably, —CH₃, and
n is, in each case independently, 0, 1, 2, or 3.

Preferred compounds of formula I are compounds 73, 276, and 285.

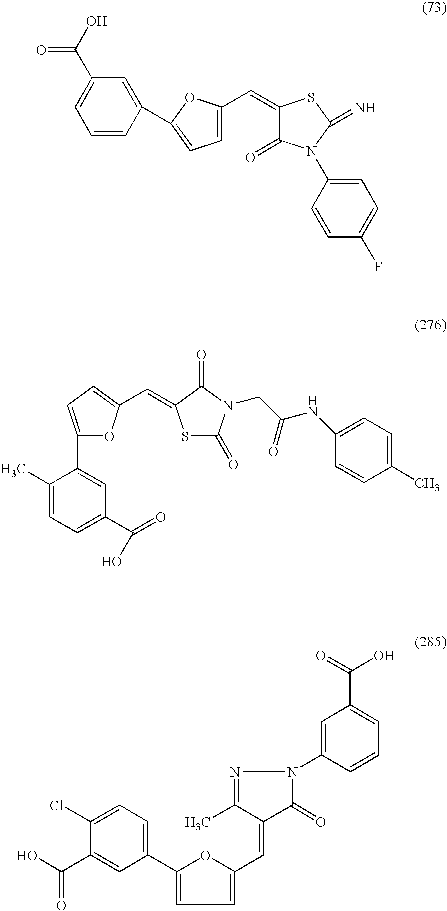

Compounds of formula II have the following formula 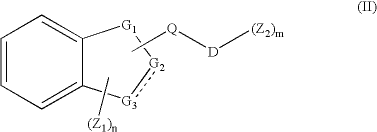
wherein,
G₁, G₂, and G₃ are, in each case independently, C, O, S, or N,
D is a phenyl ring or a 5- or 6-membered, preferably 5-membered, saturated or partially or fully unsaturated heterocyclic ring containing 1, 2 or 3, preferably, 1 or 2, heteroatoms selected from O, S, and N,
Q is a straight chain or branched alkylene or alkenylene group containing 1-5 carbon atoms, which is optionally substituted with ═O and/or —OH, preferably forming a carboxylic acid group when substituted, and in which optionally 1 or 2 carbon atoms, independently of each other, are replaced with an N or S atom,
Z₁ is, in each case independently, ═O, halogen, preferably Cl when halogen, or an alkyl group containing 1-5 carbon atoms, which is optionally substituted with ═O and/or —OH, preferably forming a carboxylic acid group when substituted,
Z₂ is, in each case independently, ═O, or an alkyl group containing 1-5 carbon atoms, which is optionally substituted with ═O and/or —OH, preferably forming a carboxylic acid group when substituted, and preferably is —CH₃, or —COOH,
n is 0, 1, or 2, and
m is 0, or 1.

Preferred compounds of formula II are 201, 213, 220, and 249.

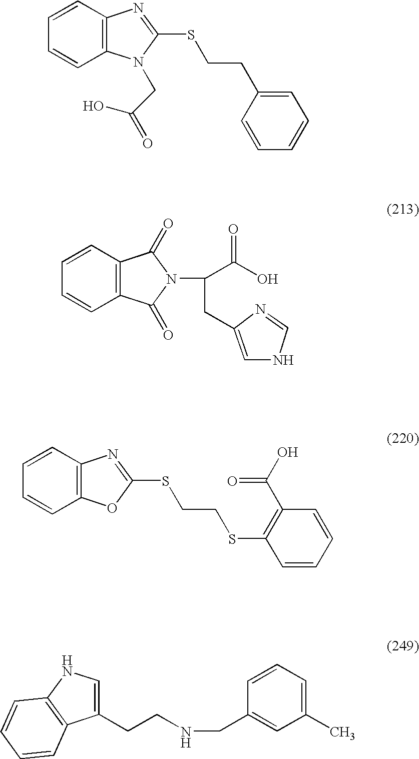

Compounds of formula III have the following formula 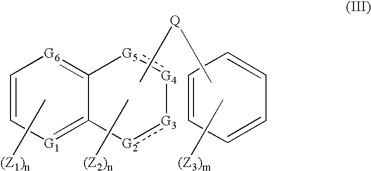
wherein,
G₁, G₂, G₃, G₄, G₅ and G₆ are, in each case independently, C, O, S, or N, such that four or five of G₁, G₂, G₃, G₄, G₅ and G₆ are C atoms and the remaining G₁, G₂, G₃, G₄, G₅ and G₆ are O, S, or N, preferably, N or O,
Q is a bond or a straight chain or branched alkylene or alkenylene group containing 1-10 carbon atoms which is optionally substituted with ═O in one or two places, and in which optionally 1 or 2 carbon atoms, independently of each other, are replaced with an N or O atom, and in which optionally a carbon atom is replaced with a 6-membered heterocyclic group containing 1 or 2 nitrogen atoms when the alkylene or alkenylene group is a straight chain group,
Z₁ is, in each case independently, —OH, halogen, preferably, Cl or Br when halogen, or an alkyl group containing 1-5 carbon atoms, preferably —CH₃ when alkyl,
Z₂ is, in each case independently, ═O, halogen, preferably, Br when halogen, or an alkyl group containing 1-10 carbon atoms which is optionally substituted with ═O in one or two places and/or —OH, preferably forming a carboxylic acid when substituted, and in which optionally 1 or 2 carbon atoms, independently of each other, are replaced with an N and/or S atom,
Z₃ is, in each case independently, —OH, halogen, preferably Br or Cl when halogen, —NO₂, an alkyl group containing 1-5 carbon atoms, in which optionally a carbon atom is replaced with an O atom, and which is optionally substituted with ═O in one or two places, preferably —O—C, —O—C—C, or —C(O)—O—C when such an alkyl, or is —O-phenyl, wherein the phenyl group in the —O-phenyl is optionally 'substituted with an —NO₂ group,
n is 0, 1, or 2, and
m is 0, 1, 2, or 3.

Preferred compounds of formula III are 92, 139, 195, and 264.

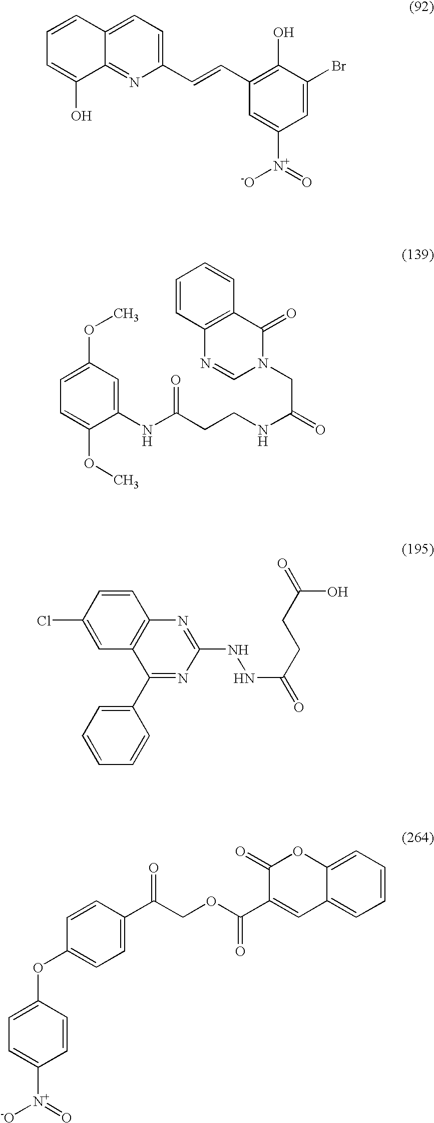

Compounds of formula IV have the following formula 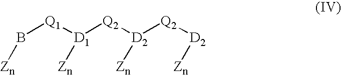
wherein,
B is a phenyl ring,
D₁ is a phenylene ring or a 6-membered saturated or partially or fully unsaturated heterocyclic ring containing 1, 2, or 3 heteroatoms selected from O, S, and N, preferably N,
D₂ and D₂′ are, each independently of each other, absent or a phenyl or phenylene ring or a 5-or 6-membered, preferably 5-membered, saturated or partially or fully unsaturated heterocyclic ring containing 1, 2 or 3, preferably, 1 or 2, heteroatoms selected from O, S, and N, preferably S, and D₂′ is preferably absent,
Q₁ is a bond or a branched or straight chain alkylene or alkenylene group containing 1-10 carbon atoms, which is optionally substituted with 1 to 5 ═O and/or OH groups, in which optionally 1, 2, or 3 carbon atoms are, in each case independently, replaced with an N, O or S atom, wherein S is optionally substituted with 1 or 2 ═O groups,
Q₂ and Q₂′ are, each independently of each other, a bond or a branched or straight chain alkylene group containing 1-5 carbon atoms, which is optionally substituted with an ═O group, in which optionally a carbon atom is replaced with an N, S, or O atom, preferably with an N atom, and preferably contains a —C(O)NH— group, wherein Q₂ is absent when D₂ is absent and Q₂′ is absent when D₂′ is absent,
Z is, in each case independently, ═O, ═S, —OH, —NH₂, —NO₂, —C N, —SO₃H, is halogen, preferably F, Cl or Br when halogen, or a straight chain or branched alkyl or alkenyl group containing 1 to 10, preferably 1 to 6 carbon atoms, which is optionally substituted with 1 to 3 ═O and/or OH groups, preferably forming a carboxylic acid group when substituted, and in which optionally a carbon atom is replaced with an N, O or S atom, preferably with an N or S atom, and is preferably, —N—C(O)—C═C—COOH, —N—C(O)—C—C—COOH, —S—C—COOH, —N—C(O)—C—, —CH₃, —COOH or —OCH₃, more preferably, —CH₃ or —COOH, when such an alkyl group, or is a cyclic alkyl group containing 3 carbon atoms,
n is, in each case independently, 0, 1, 2, 3, 4 or 5, preferably, 0, 1, 2 or 3, or preferably up to 4 or 5 when some of the S groups are halogen.

Preferred compounds of formula IV are 88, 99, 146, 160, 172, 196, 248, 259, 261, and 262.

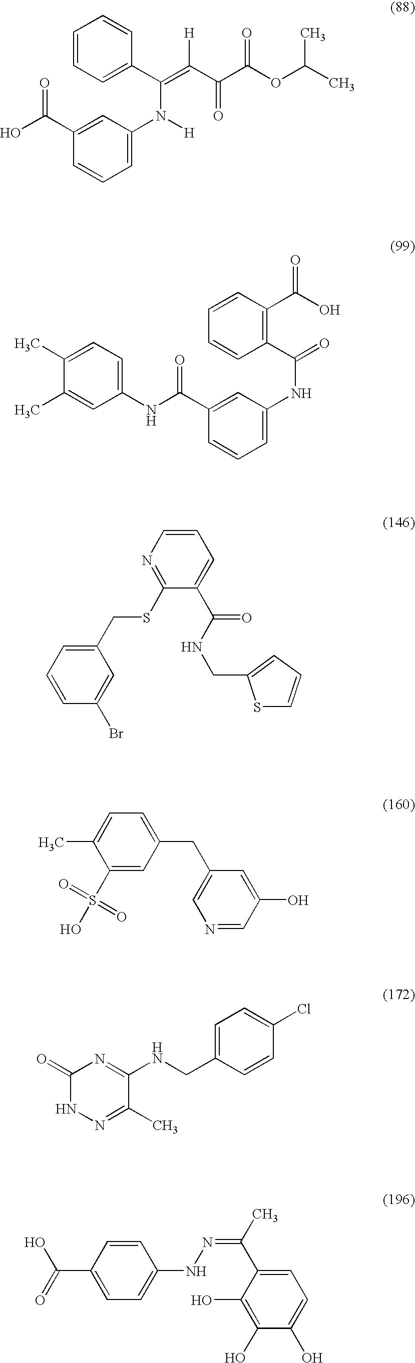

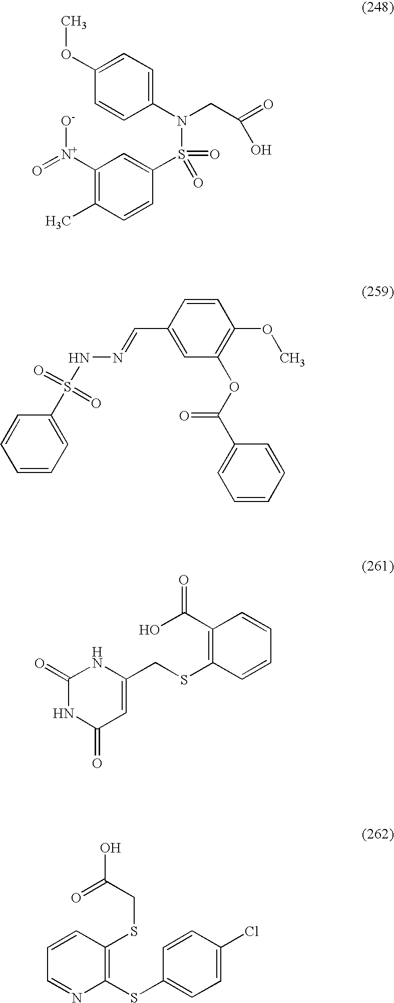

Compounds of formula V have the following formula 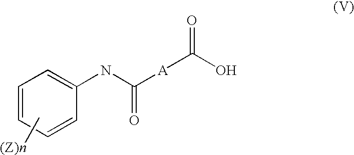
wherein,
Z is, in each case independently, —NO₂, an alkyl containing 1 to 5 carbon atoms, in which optionally a carbon atom is replaced with an O atom, and which is optionally substituted with an ═O group, and is preferably CH₃, —C(O)—O—C or —COOH when such an alkyl,
A is a straight chain alkylene group containing 1 to 5 carbon atoms, preferably 2 or 3 carbon atoms, and
n is 1, 2 or 3.

Preferred compounds of formula V are 162 and 199.

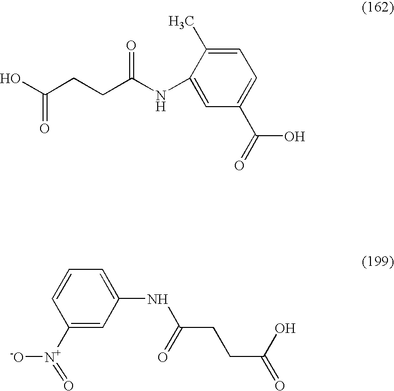

Compounds of formula VI have the following formula 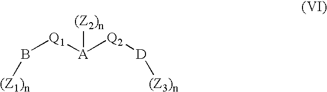
wherein,
B is a phenyl ring,
D is absent, or is a phenyl ring or a 6-membered saturated or partially or fully unsaturated heterocyclic ring containing 1, 2, or 3 heteroatoms selected from O, S, and N, preferably N,
A is a 5-membered saturated or partially or fully unsaturated heterocyclic ring containing 1, 2, 3 or 4 heteroatoms selected from O, S, and N,
Q₁ and Q₂ are, in each case independently of each other, a bond or a straight chain or branched alkylene group containing 1-5 carbon atoms, in which optionally a carbon atom is replaced with an O, N or S atom, and in which optionally 1 or 2 —C— groups are replaced with —C═ or ═C— groups (said —C═ and/or ═C— group still subject to the previously mentioned replacement with and N or S atom, for example), and which is optionally substituted with an ═O group, wherein Q₂ is absent when D is absent,
Z₁ is, in each case independently, —NO₂, —OH, halogen, preferably Cl, Br or F when halogen, or an alkyl group containing 1-5 carbon atoms, which is optionally substituted with up to 3 ═O and/or OH groups, preferably forming a carboxylic acid group when substituted, and in which optionally a carbon atom is replaced with and N, S or O atom, and is preferably —CH₃, —OCH₃ or —COOH when such an alkyl group,
Z₂ is, in each case independently, —NH₂, —OH, ═NH, ═O, ═S, phenyl, halogen, preferably Cl when halogen, or an alkyl group containing 1-5 carbon atoms, which is optionally substituted with up to 3 ═O and/or OH groups, preferably forming a carboxylic acid group when substituted, and in which optionally a carbon atom is replaced with an S atom, and is preferably —SCH₂COOH or —CH₂COOH when such an alkyl group,
Z₃ is, in each case independently, ═O, —OH, NO₂, NH₂, halogen, preferably F or Br when halogen, or an alkyl group containing 1-5 carbon atoms, which is optionally substituted with up to 3 ═O and/or OH groups, preferably forming a carboxylic acid group when substituted, and in which optionally a carbon atom is replaced with an O atom, and
n is, in each case independently, 0, 1, 2 or 3.

Preferred compounds of formula VI are 103, 222, 239, 254, 275 and 287.

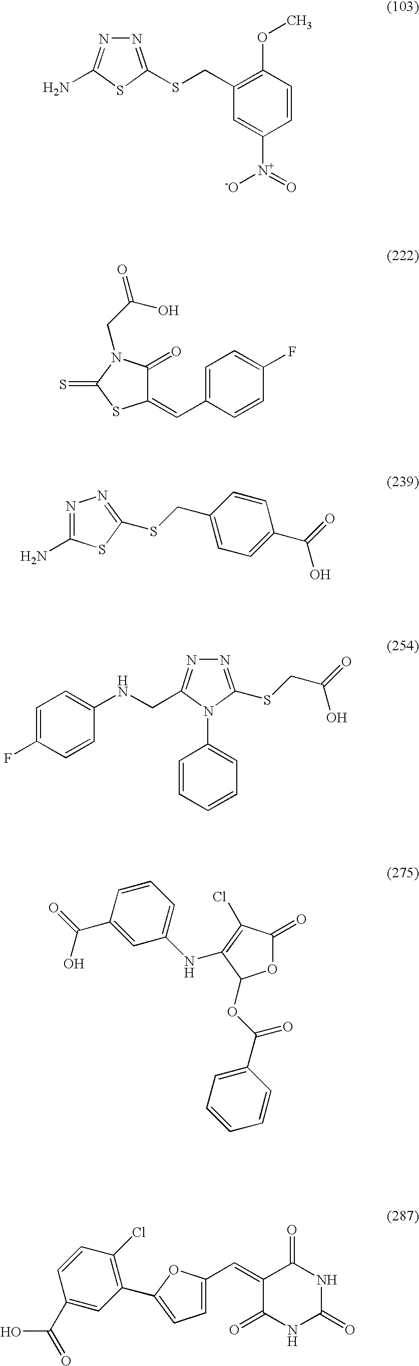

Compotnds of formula VII have the following formula 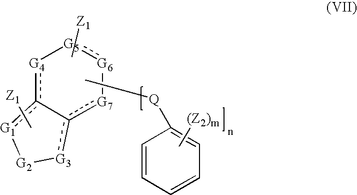
wherein,
G₁ to G₇ are, in each case independently, C, O, S, or N, preferably, C, N or S, wherein at least 3 of G₁ to G₇ are C atoms,
Z₁ is, in each case independently, absent, or ═O, ═NH or an alkyl group containing 1 to 5 carbon atoms, and is preferably —CH₃ when an alkyl group,
Z₂ is, in each case independently, a straight chain or branched, preferably straight chain, allyl group containing 1 to 5 carbon atoms, in which optionally a carbon atom is replaced with an O atom, and which is optionally substituted with 1 or 2 ═O and/or —OH groups, preferably forming a carboxylic acid group when substituted, and is preferably —CH₃, —OCH₃, —OCC or —COOH when such an alkyl,
Q is, in each case independently, a bond or an alkylene group containing 1-5 carbon atoms, which is optionally substituted with ═O, in which optionally 1, 2, or 3 carbon atoms are, in each case independently, replaced with an N or S atom, wherein S is optionally substituted with 1 or 2 ═O groups, and
n is 0, 1 or 2, and
m is 1 or 2.

Preferred compounds of formula VII are 105 and 149.

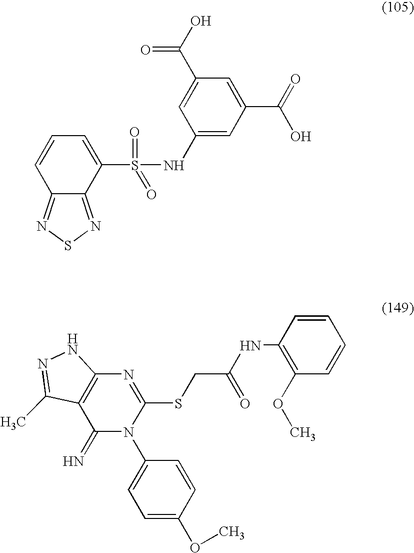

Compounds of formula VIII have the following formula 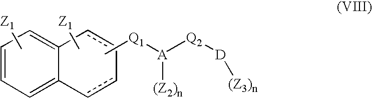
wherein,
A is a 5-membered saturated or partially or fully unsaturated heterocyclic ring containing 1, 2 or 3, preferably 2, heteroatoms selected from O, S, and N, preferably selected from S and N, or is a C₁₀ aromatic bi-cyclic ring containing 1, 2 or 3, preferably, 1, heteroatoms selected from O, S, and N, preferably N,
D is absent or is a fully or partially saturated or unsaturated cyclic ring containing 6 or 7 carbon atoms, and is preferably phenylene,
Q₁ and Q₂ are, each independently of each other, a bond or a straight chain or branched alkylene group containing 1-10, preferably 1-5, carbon atoms, which is optionally substituted with an ═O group, and in which optionally 1, 2 or 3, preferably 2, carbon atoms, independently of each other, are replaced with an N or O atom, and wherein optionally 1-3 carbon atoms are replaced with a —C═ and/or ═C— (said —C═ and/or ═C—group still subject to the previously mentioned replacement with and N atom, for example), and/or when the alkylene group is straight chain with a phenyl group, preferably with a —C═ or ═C— group, wherein Q₂ is absent when D is absent,
Z₁ is, in each case independently, absent or an alkyl group containing 1 to 5 carbon atoms, in which optionally a carbon atom is replaced with an —O— group, and which is optionally substituted with one or two ═O or —OH groups, preferably forming a carboxylic acid group when substituted,
Z₂ is, in each case independently, ═O or an alkyl group containing 1-5 carbon atoms, which is optionally substituted with ═O in one or two places and/or —OH, preferably forming a carboxylic acid when substituted,
Z₃ is halogen, preferably Cl or F when halogen, or an alkyl group containing 1 to 5 carbon atoms, which is optionally halogenated, preferably is —CF₃ when such a halogenated alkyl group, and
n is 1 or 2.

Preferred compound of formula VIII is 245.

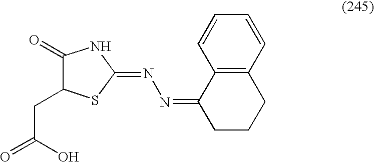

Compounds of formula IX have the following formula 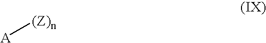
wherein,
A is a 5- or 6-membered saturated or partially or fully unsaturated heterocyclic ring containing 2 or 3 heteroatoms selected from S and N,
Z is, in each case independently, a straight chain or branched alkyl group containing 3-5 carbon atoms, which is substituted with ═O and/or OH groups, preferably forming a carboxylic acid group when substituted, and in which a carbon atom is replaced with an S atom, and is preferably —SCH₂COOH, and
n is 1, 2, or 3, preferably 2.

Preferred compound of formula IX is 232.

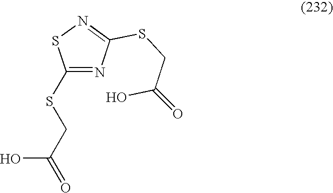

Compounds of formula X have the following formula 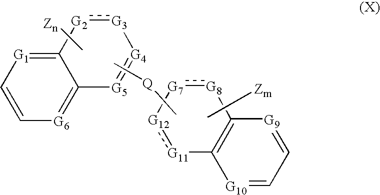
wherein,
G₁ to G₁₂ are, each independently of each other, C, N, S or O, preferably 8 or 10 of G₁ to G₁₂ are carbon atoms and the remaining of G₁ to G ₁₂ are N atoms,
Z is, in each case independently, an allyl containing 1 to 5 carbon atoms, preferably 1 carbon atom, which is optionally substituted with 1 to 2 ═O and/or —OH groups, preferably forming a carboxylic acid group when substituted,
Q is a bond or an alkylene group containing 1 to 5 carbon atoms,
m 0, 1, 2or 3,
n 0, 1, 2 or 3, such that
m+n 1.

Preferred compound of formula X is 197.

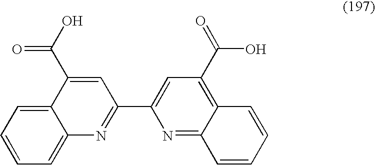

Compounds of formula XI have the following formula 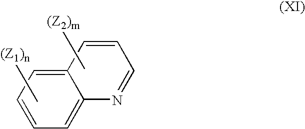
wherein,
Z₁ is, in each case independently, halogen, preferably Cl when halogen, —NO₂ or —OH,
Z₂ is, in each case independently, an alkyl group containing 1-5 carbon atoms, which is optionally substituted with an ═O and/or —OH group, preferably forming a carboxylic acid when substituted, and in which optionally a carbon atom is replaced with an S atom, and is preferably is an —S—C—COOH group,
n is 0, 1, 2, or 3,
m is 0, 1, 2, or 3, and
n+m is 3 or more.

Compounds of formula XII have the following formula 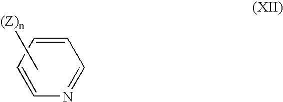
wherein,
Z is, in each case independently, —C N, halogen, preferably Cl when halogen, or an alkyl group containing 1-5 carbon atoms, which is optionally substituted with halogen, preferably up to three times with F when substituted with halogen, and/or is substituted with one or more ═O and/or —OH groups, preferably forming a carboxylic acid when substituted, and in which optionally a carbon atom is replaced with an S atom, preferably is an —S—C—COOH group, and
n is 2, 3, 4 or 5.

Compounds of formula XIII have the following formula 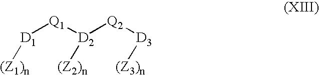
wherein,
D₁ is a 5- or 6-membered saturated or partially or fully unsaturated heterocyclic ring containing 1, 2, or 3 heteroatoms selected from O, S, and N, preferably containing 1 or 2 N,
D₂ is a 6-membered saturated or partially or fully unsaturated heterocyclic ring containing 1, 2, or 3 heteroatoms selected from O, S, and N, preferably containing 1 or 2 N, or is optionally a phenylene group when D₃ is present,
D₃ is absent or a 5- or 6-membered saturated or partially or fully unsaturated heterocyclic ring containing 1, 2, or 3 heteroatoms selected from O, S, and N, preferably containing 1 or 2 N and/or S,
Q₁ is —O—, or a straight chain alkylene group containing 1-5 carbon atoms, in which optionally a carbon atom is replaced with an N, O or S atom, preferably with S, and which is optionally substituted with an ═O atom,
Q₂ is absent when D₃ is absent or is a bond or an —O— group,
Z₁ is, in each case independently, ═O or halogen, preferably Cl or F when halogen,
Z₂ is, in each case independently, ═O, —C N, —COOH, —NO₂ or halogen, preferably Br when halogen,
Z₃ is, in each case independently, halogen, preferably Cl when halogen, and is absent when D₃ is absent, and
n is, in each case independently, 0, 1, 2, or 3.

Compounds of formula XIV have the following formula 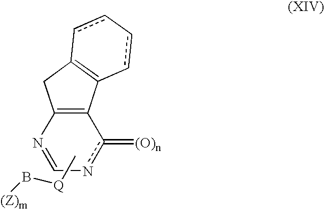
wherein,
B is a phenylene group,
Q is a straight chain alkylene group containing 1-10 carbon atoms, in which optionally up to three carbon atoms are replaced with an N, O or S atom, preferably with and N and/or S, and which is optionally substituted with 1 or 2 ═O groups,
Z is, in each case independently, halogen, preferably Cl, or an alkyl group containing 1-5 carbon atoms, in which optionally a carbon atom is replaced with an O atom,
n is 0 or 1, and
m is 1 or 2.

Compounds of formula XV have the following formula 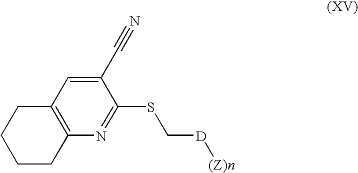
wherein,
D is, a 6-membered saturated or partially or fully unsaturated heterocyclic ring containing 1, 2, or 3 heteroatoms selected from O, S, and N, preferably containing 2 N,
Z is ═O
n is 1, or 2, preferably 2.

Compounds of formula XVI have the following formula 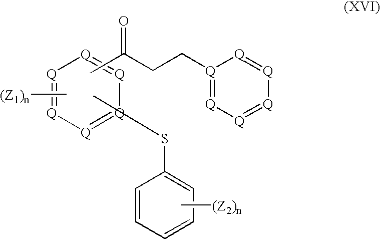
wherein,
Q is, each independently, C or N, wherein, preferably 2 of Q are N groups,
Z₁ is a phenyl group, or 2 of Z₁ together form with the Q atoms to which they are bound a 6-membered aromatic ring containing only C atoms,
Z₂ is halogen, preferably Cl, and
n is 1, or 2.

Compounds of formula XVII have the following formula 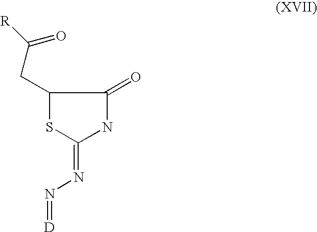
wherein,
D is, a carbocyclic group containing 8 to 10 carbon atoms, and is preferably an unsaturated 8-membered monocycle or is adamantine, and
R is —OH or an alkyl group containing 1 to 5 carbon atoms, in which optionally a carbon atom is replaced with an N or O atom or with a phenyl group, and which is optionally substituted with 1 to 2 ═O and/or —OH groups, preferably forming a carboxylic acid group when substituted.

Preference is given to compounds in all the formulae herein whose molecular weight is less than 500 Daltons.

The following known compounds, by way of example, without limiting the invention are included.

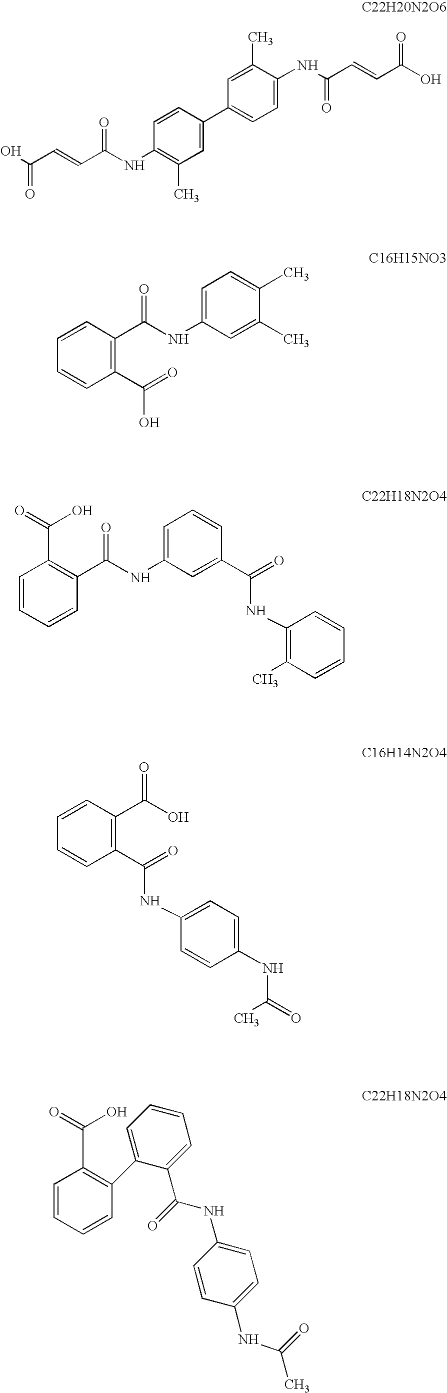

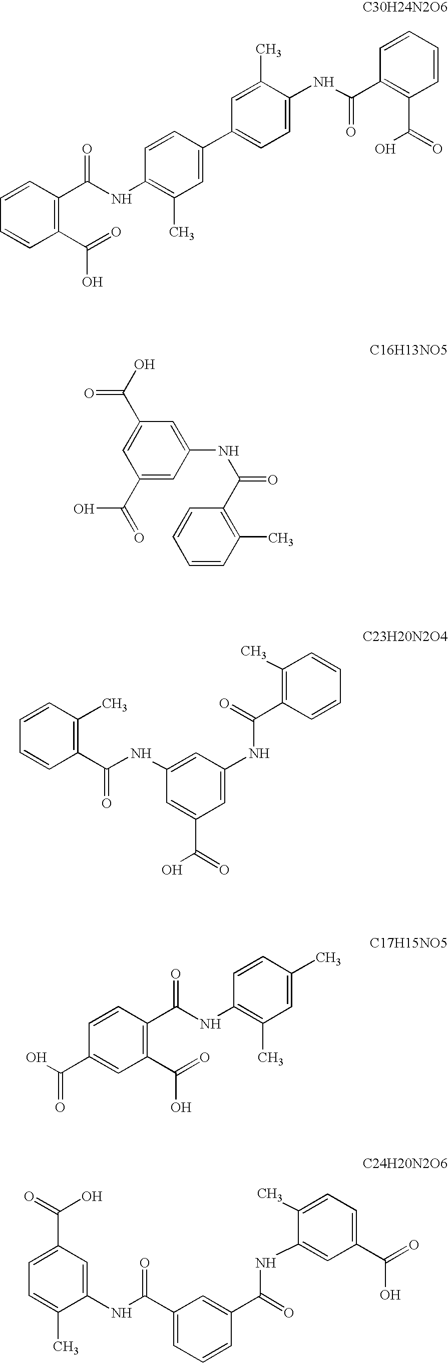

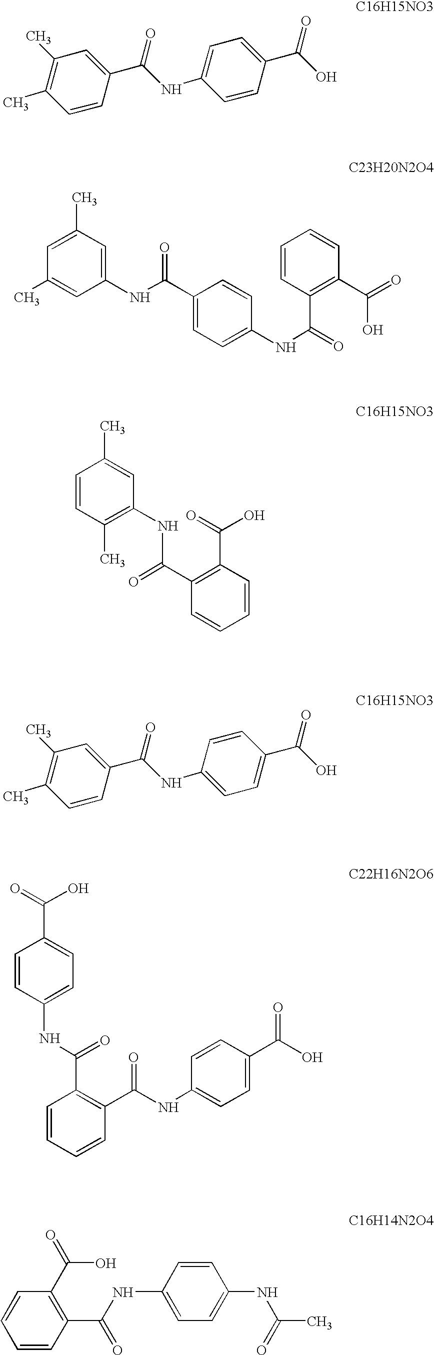

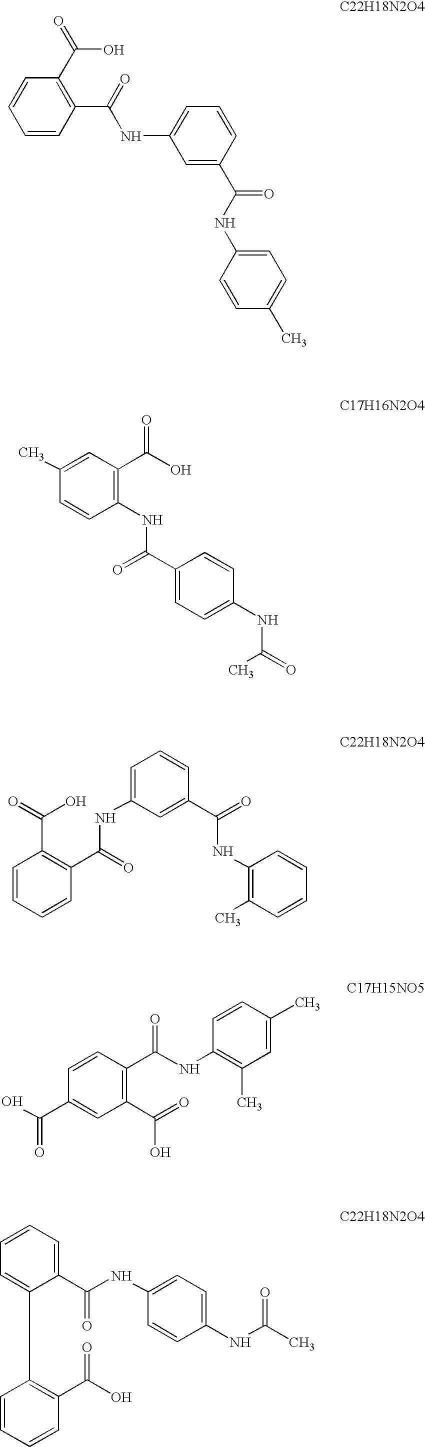

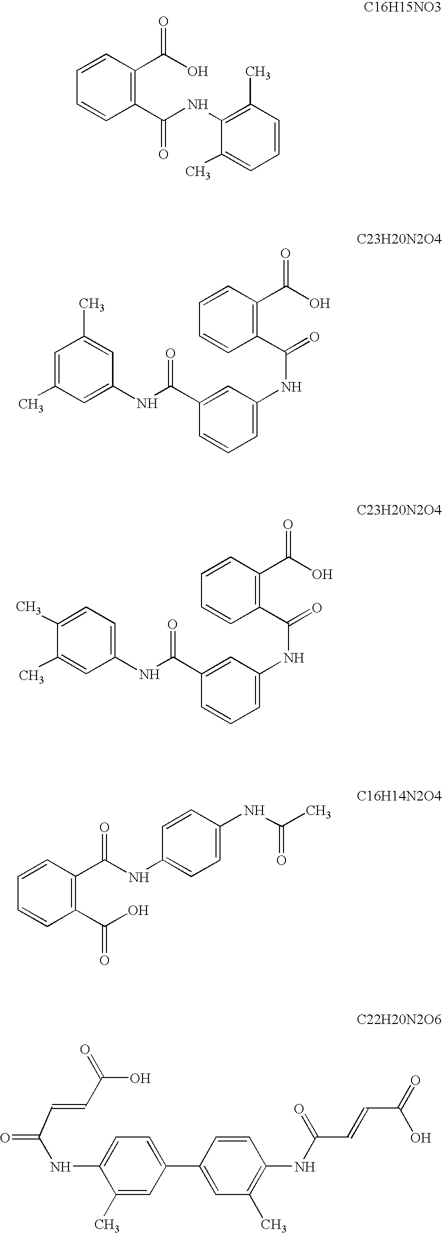

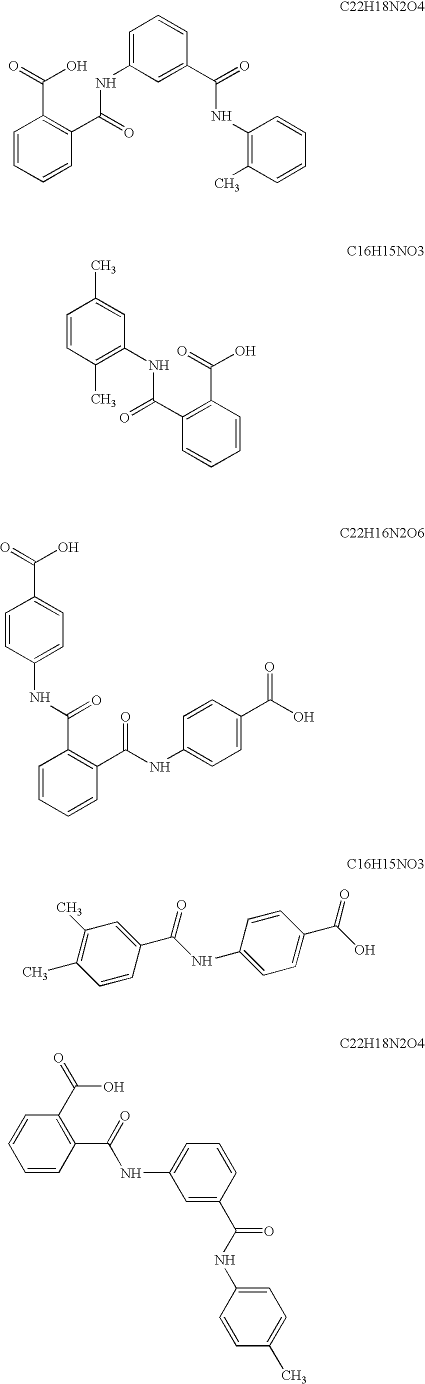

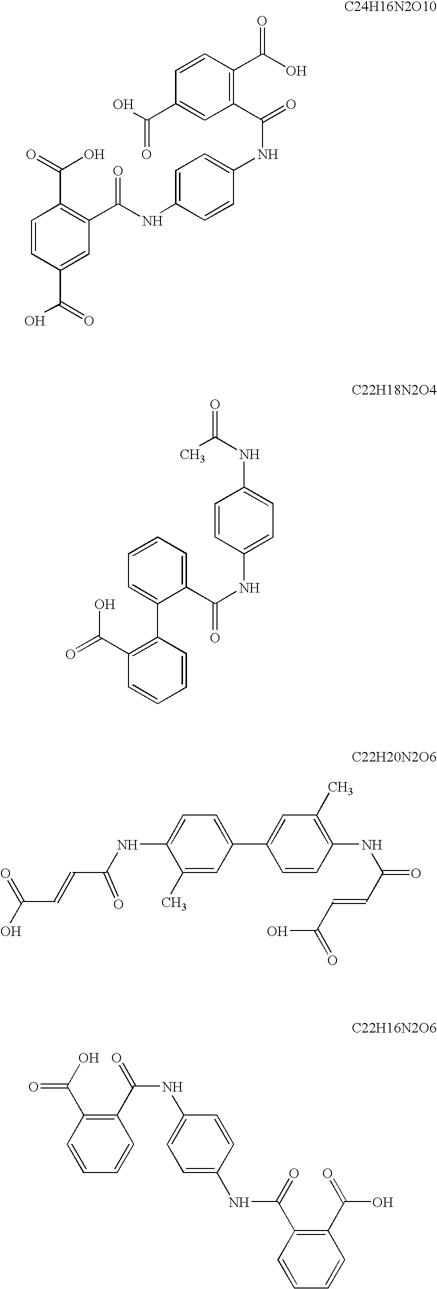

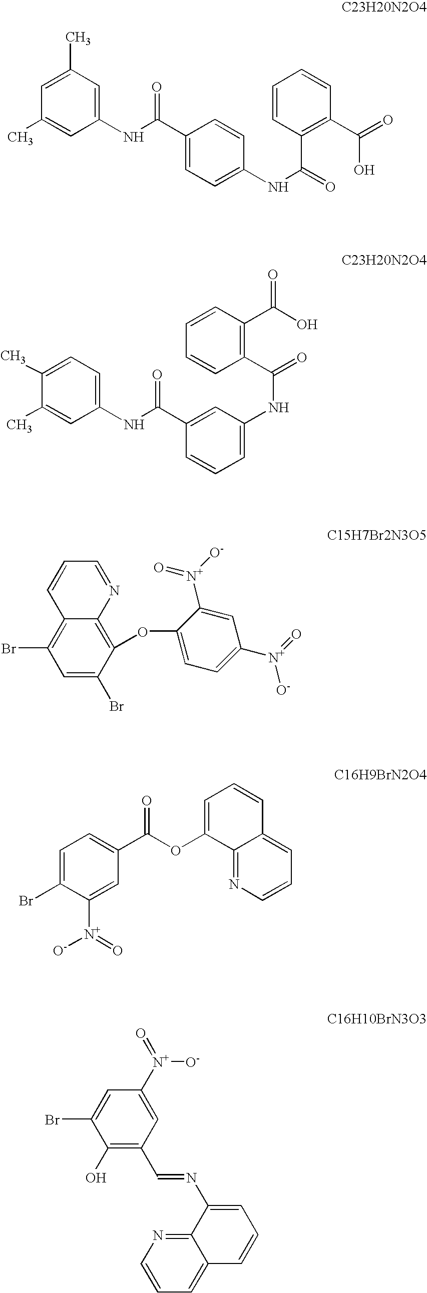

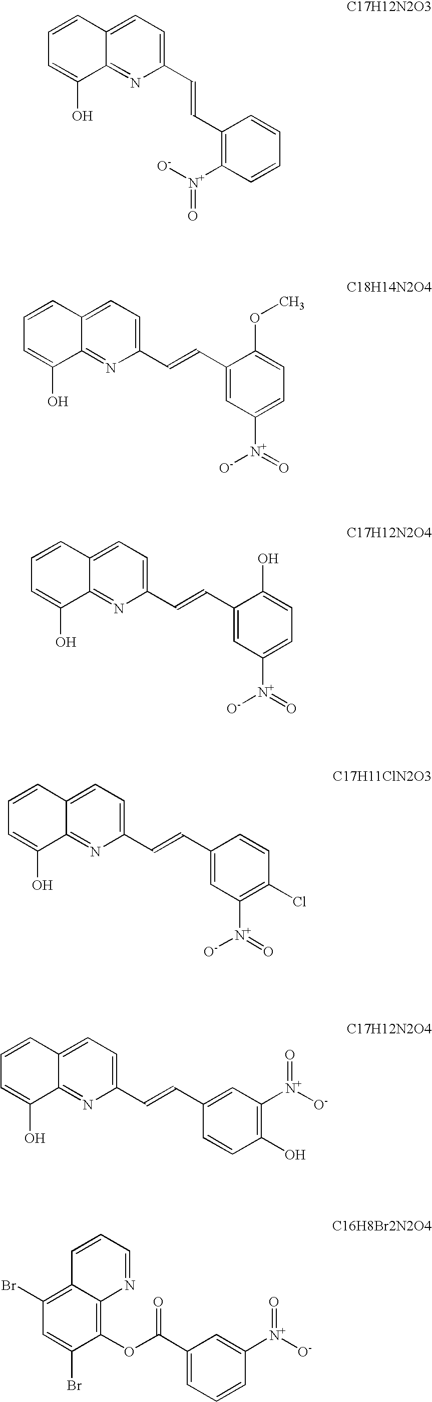

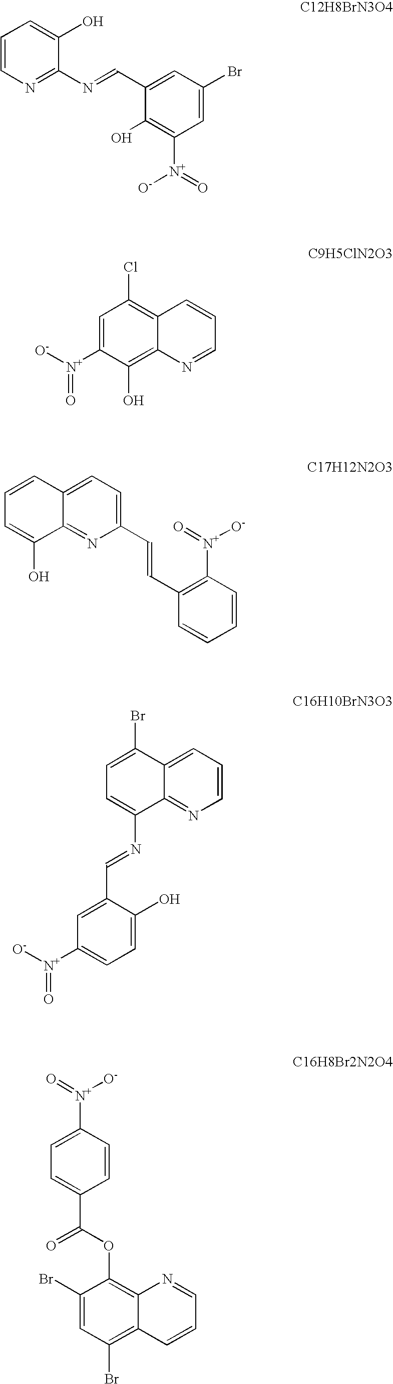

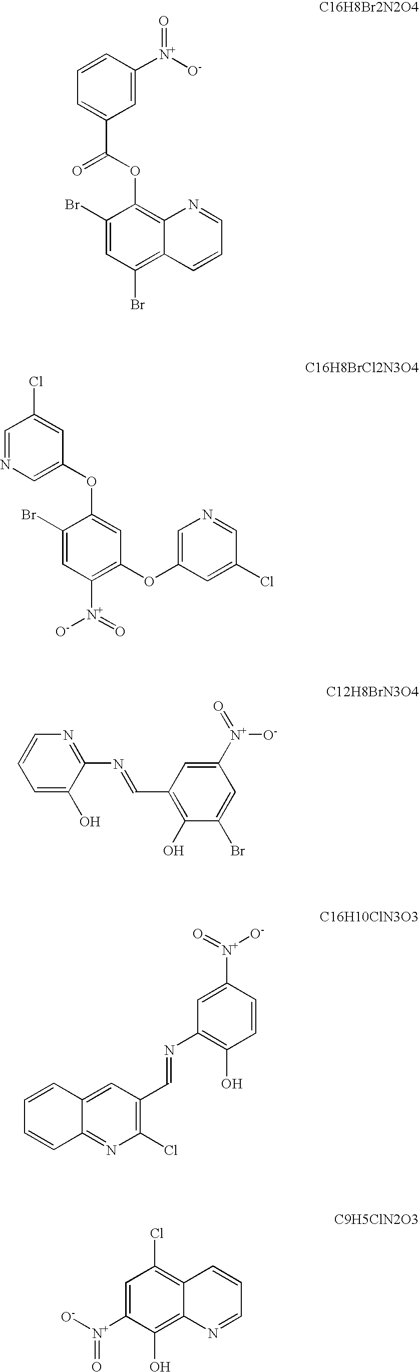

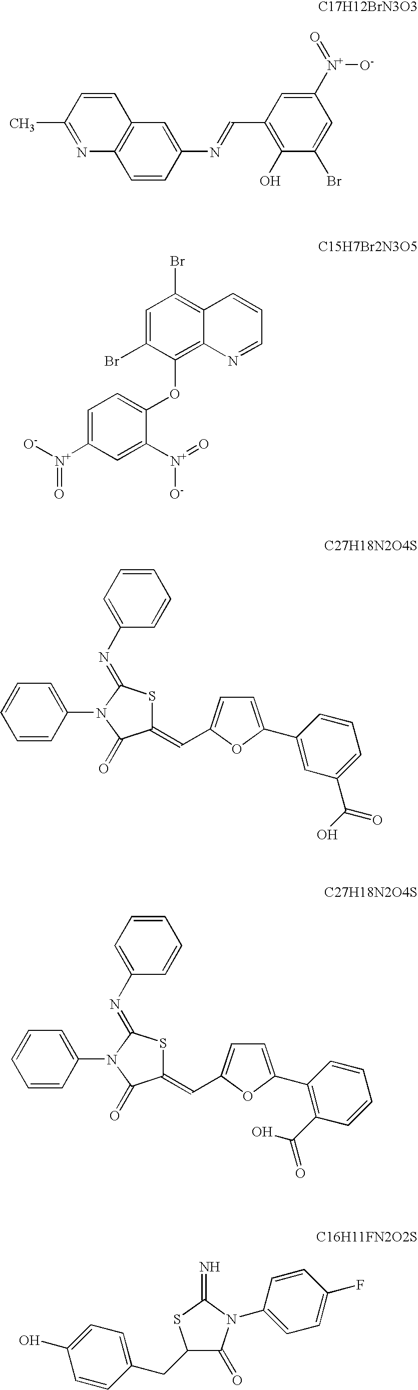

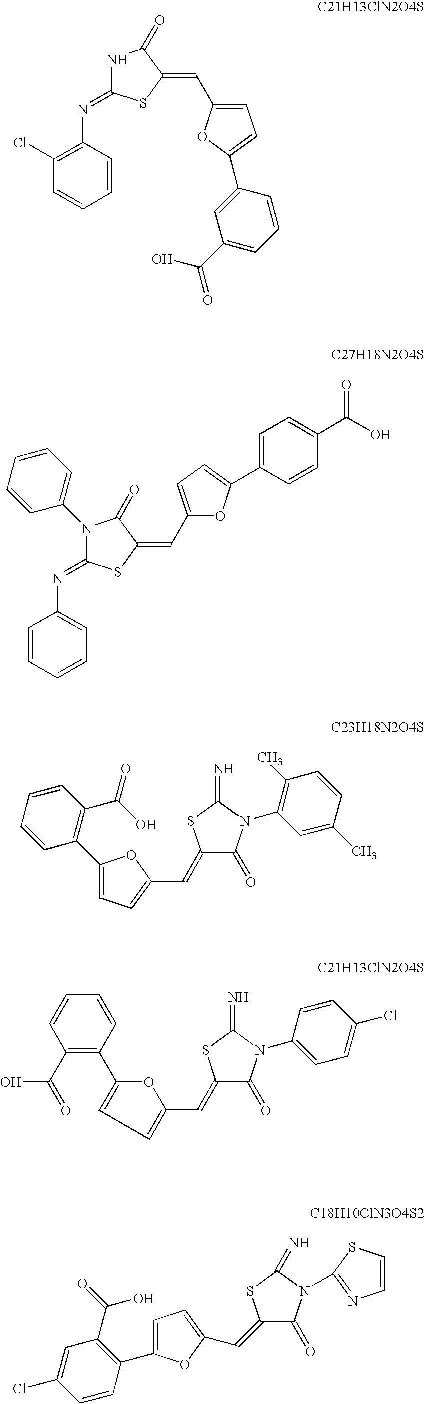

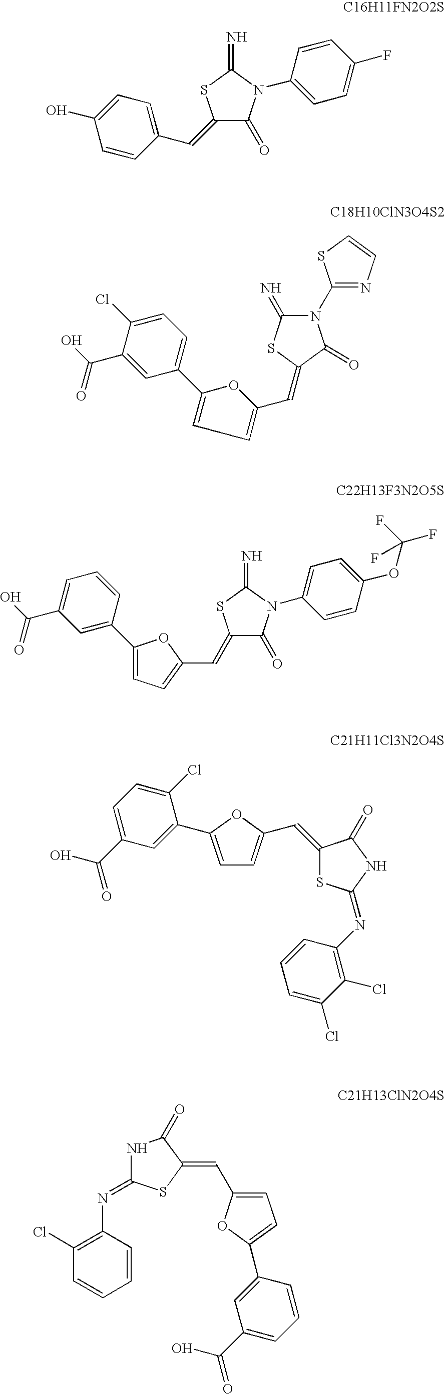

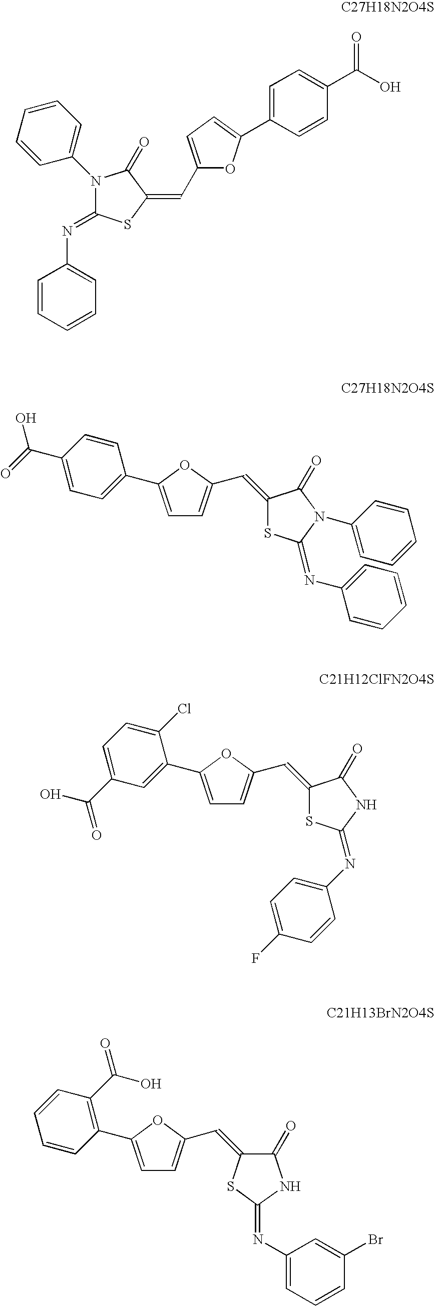

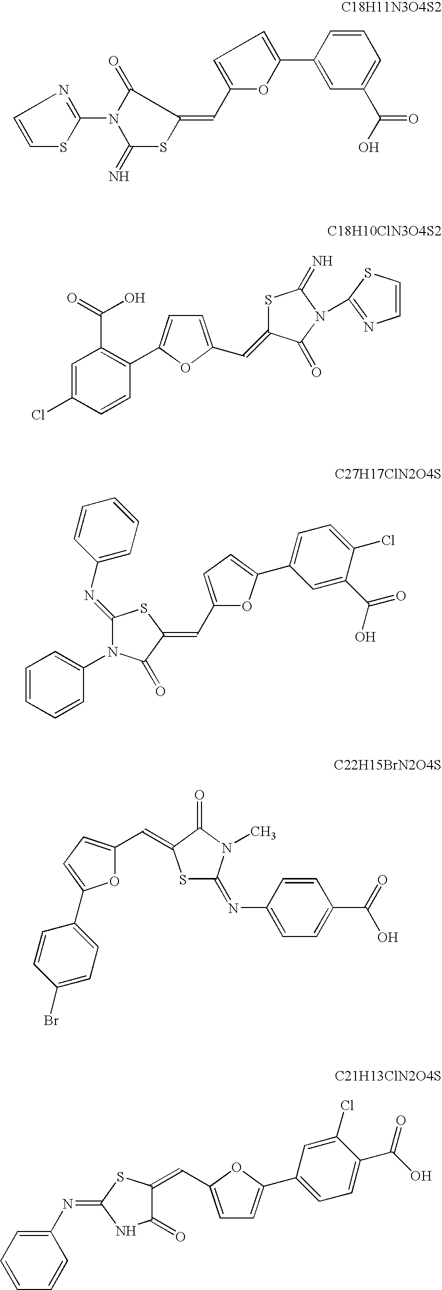

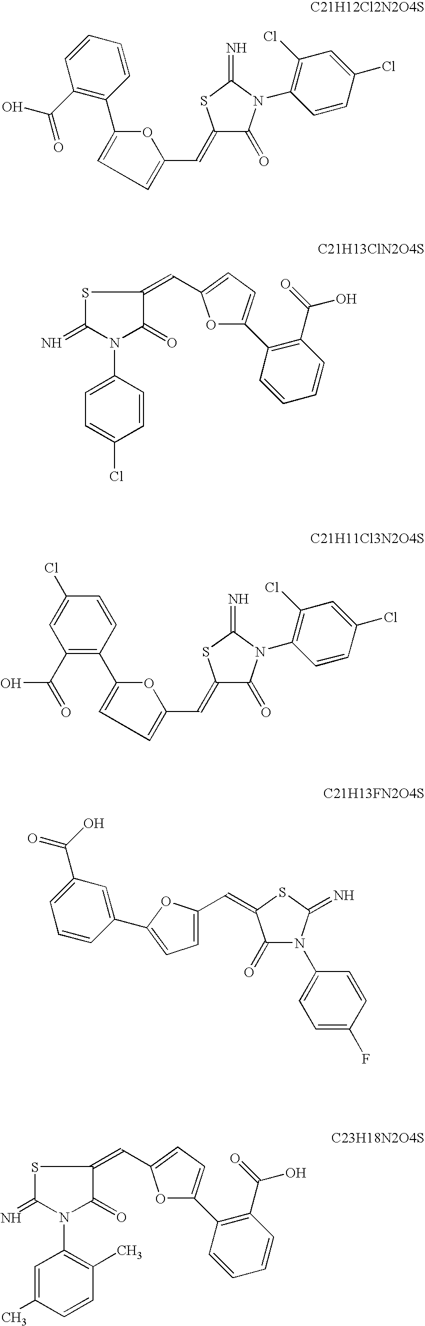

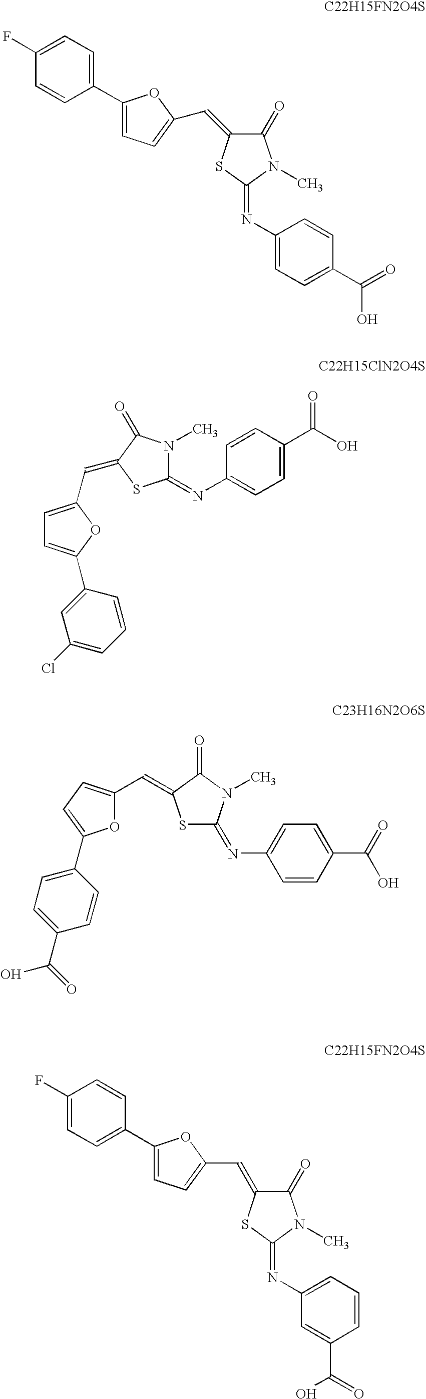

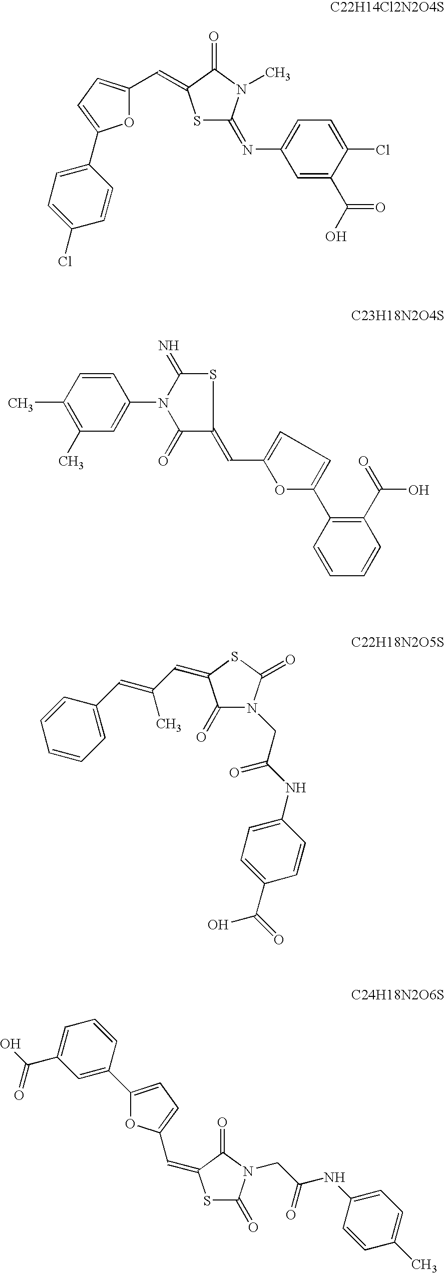

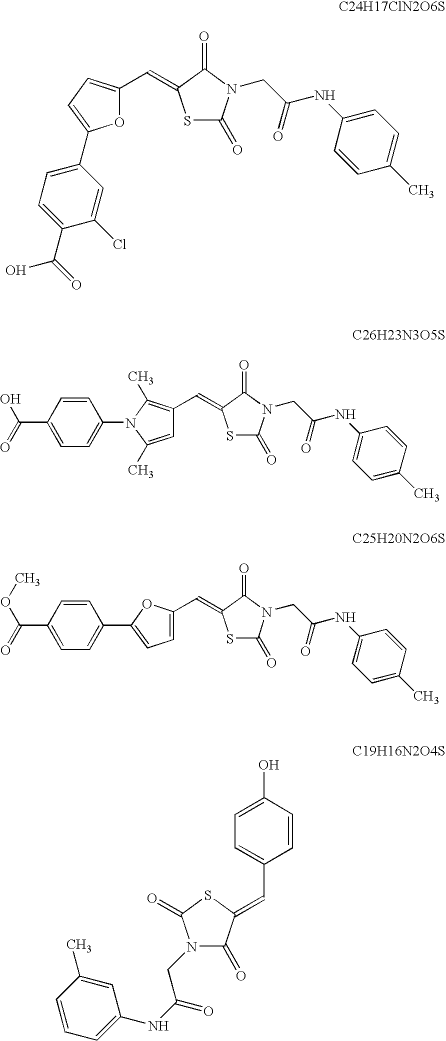

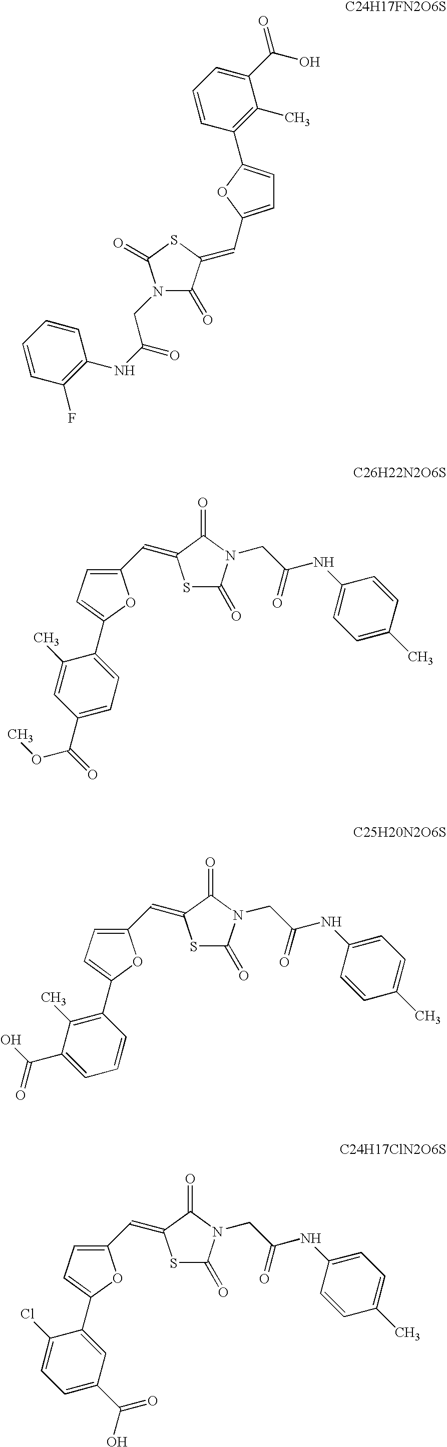

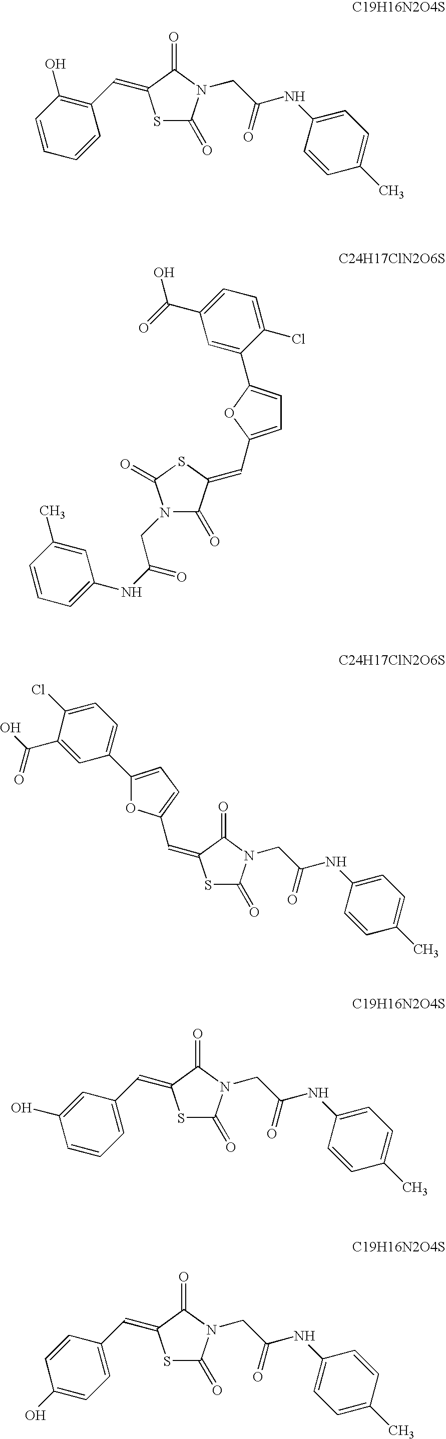

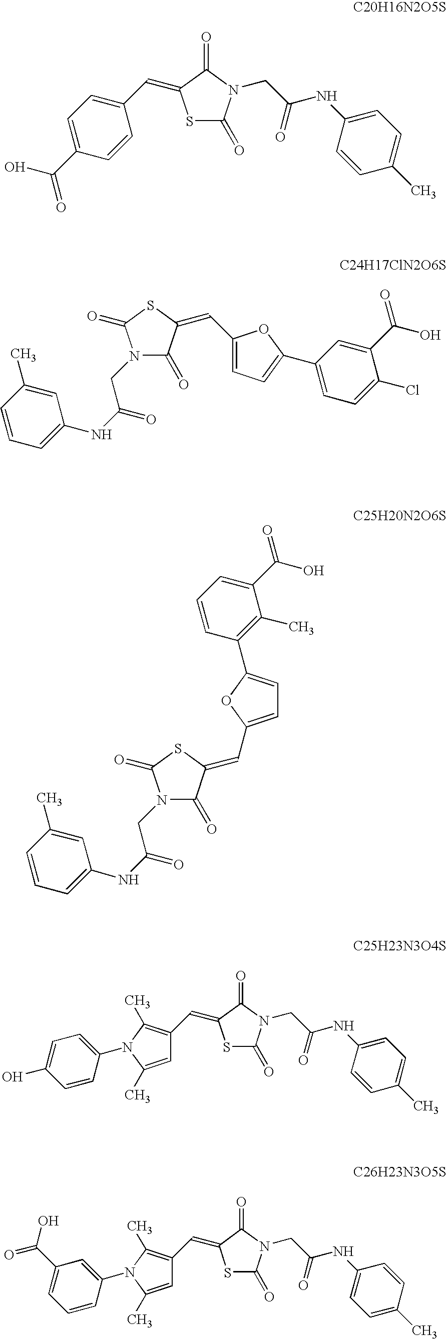

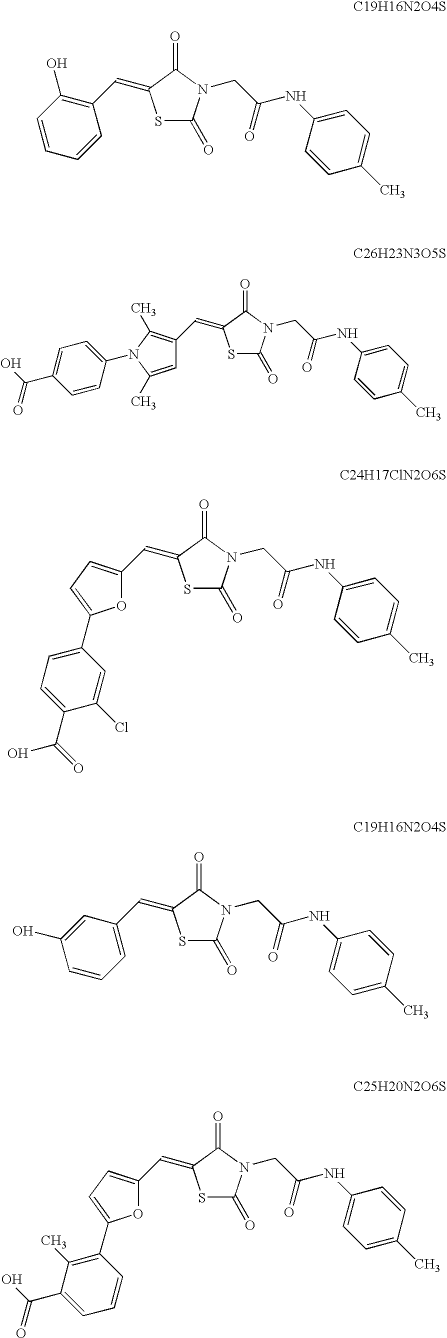

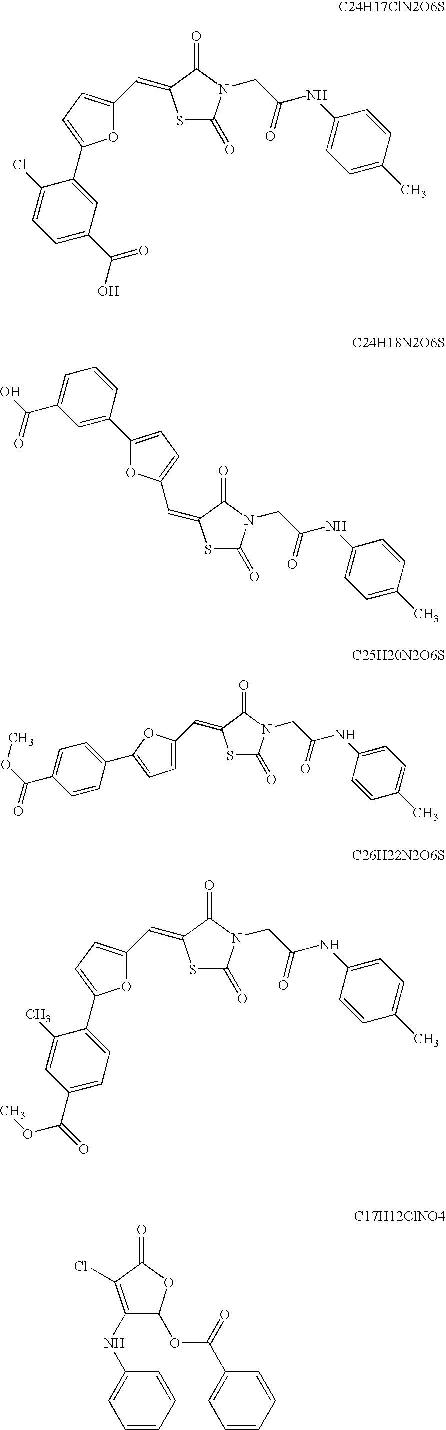

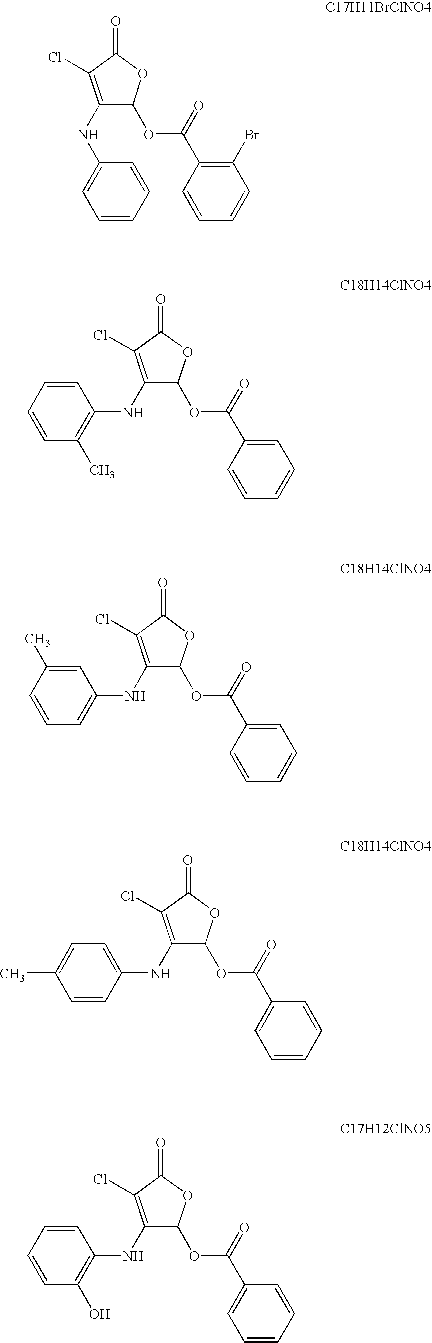

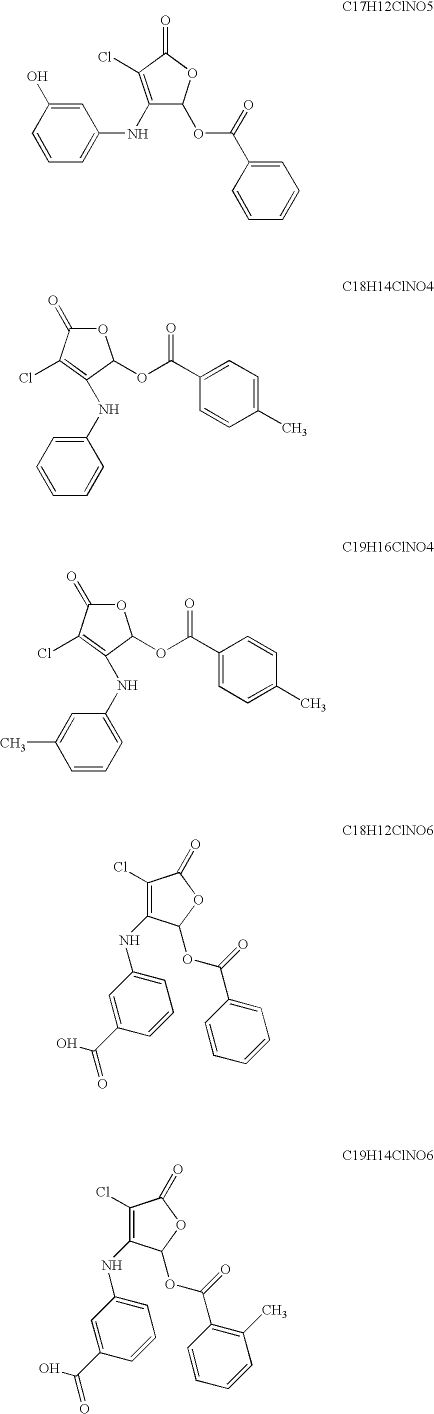

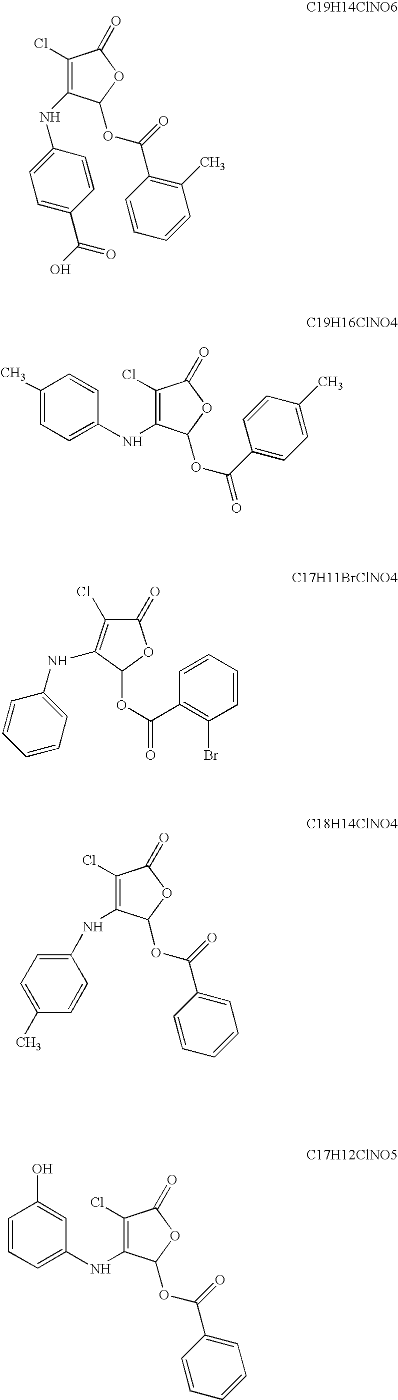

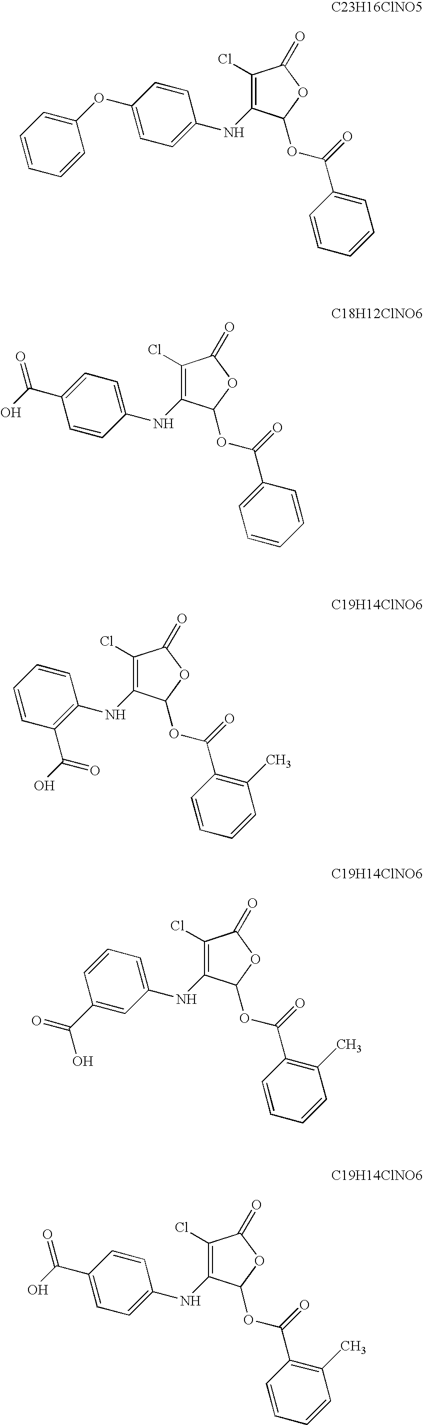

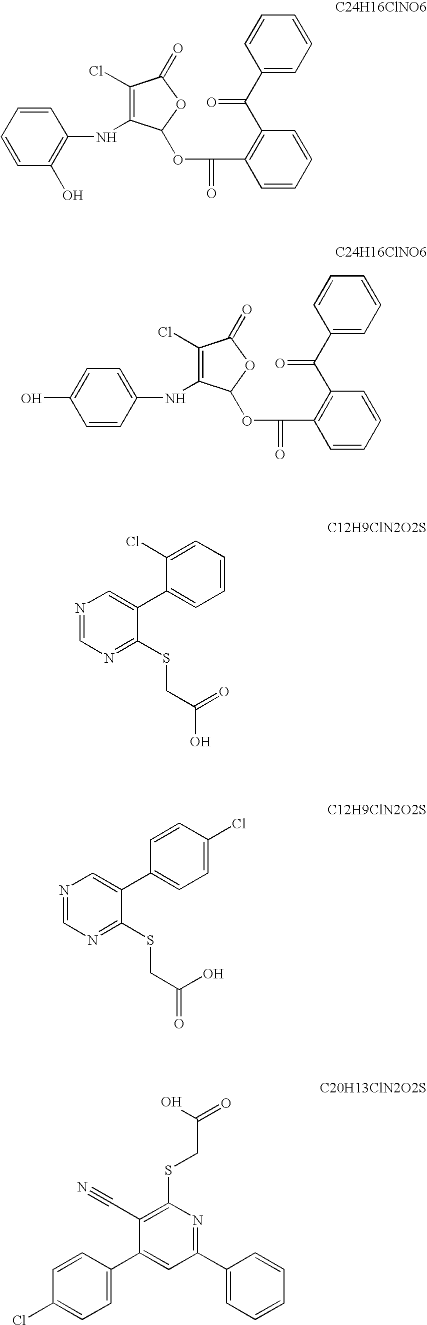

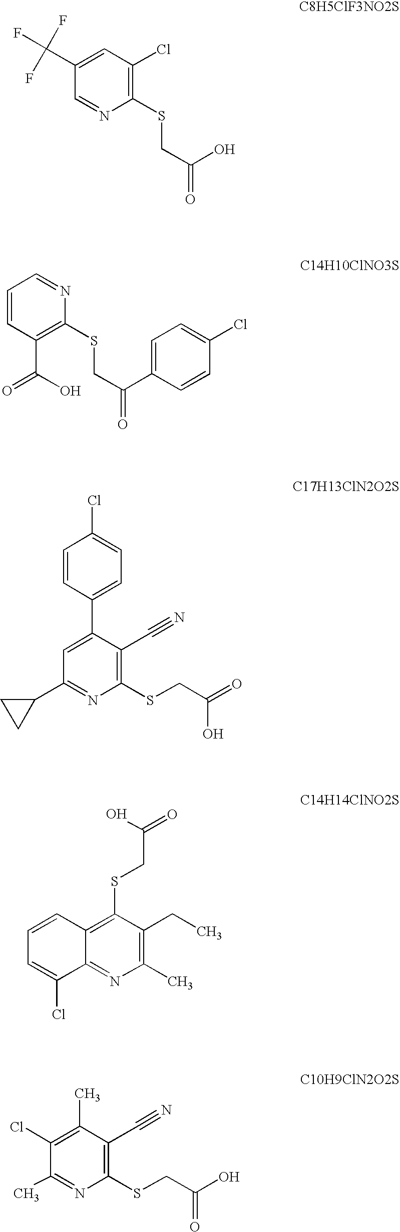

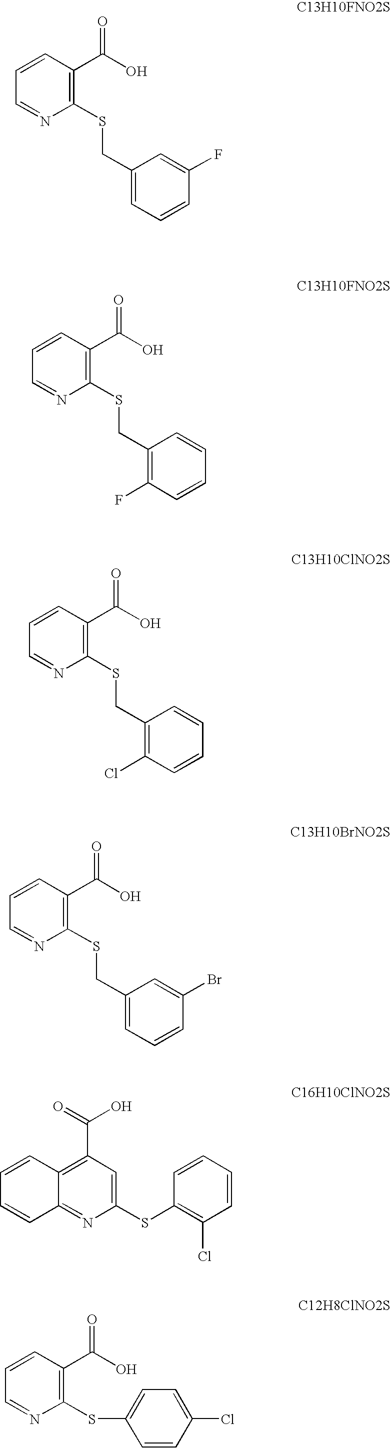

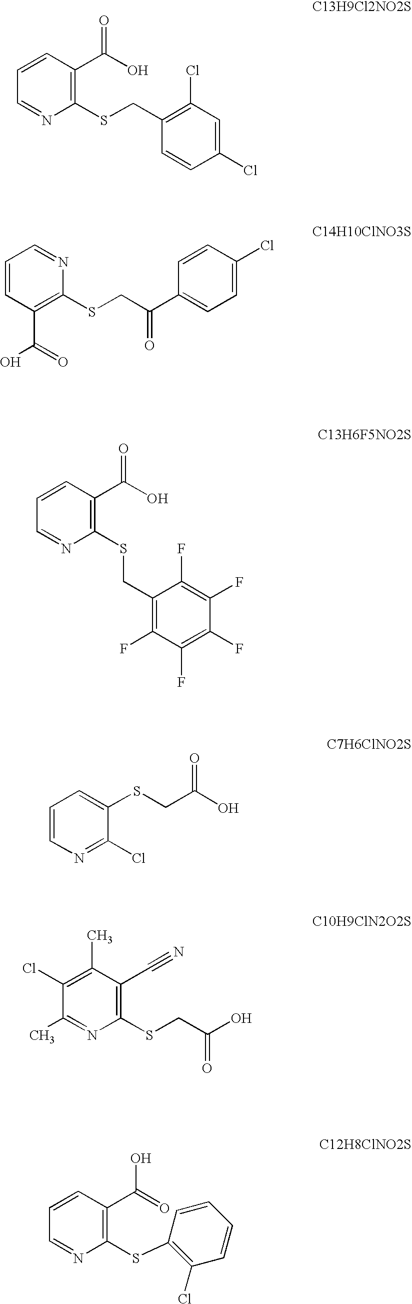

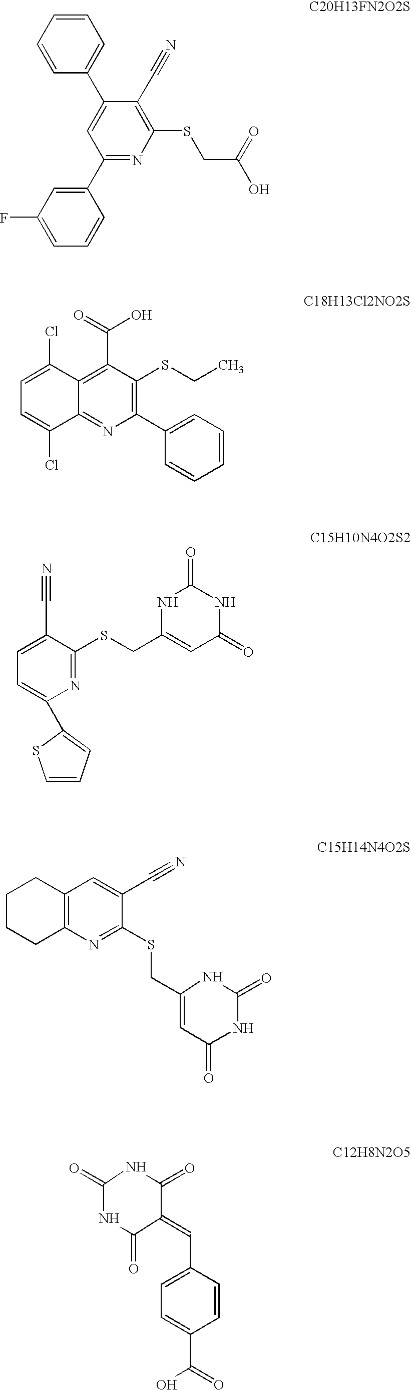

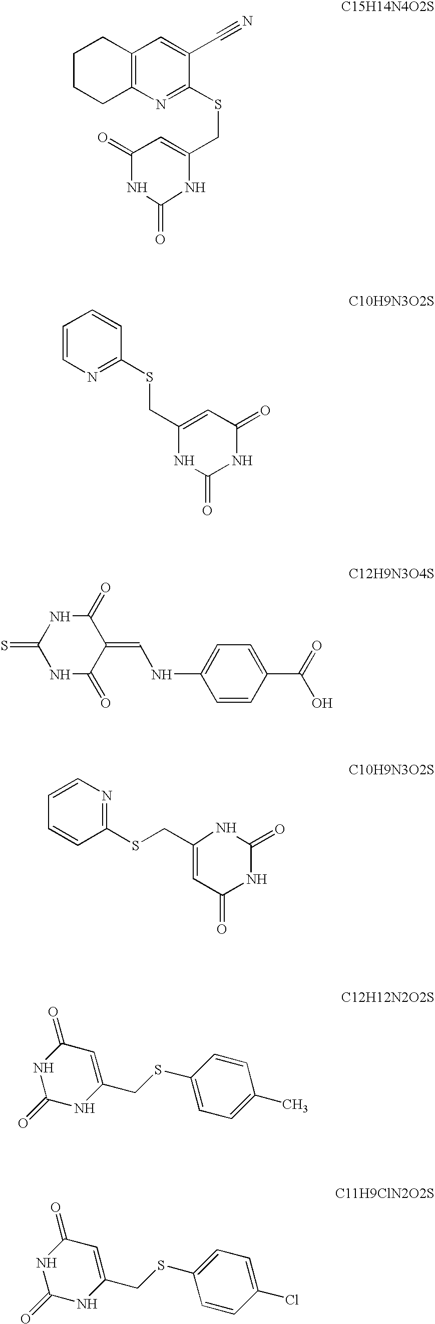

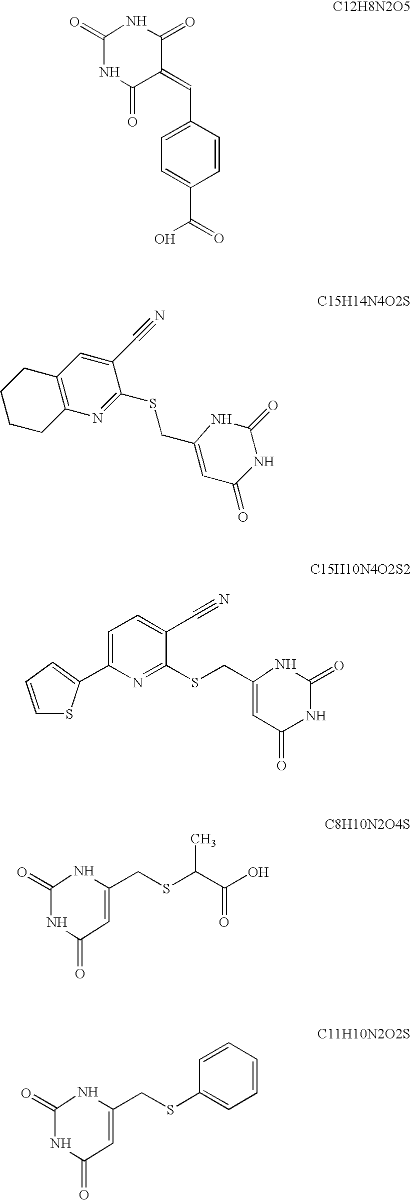

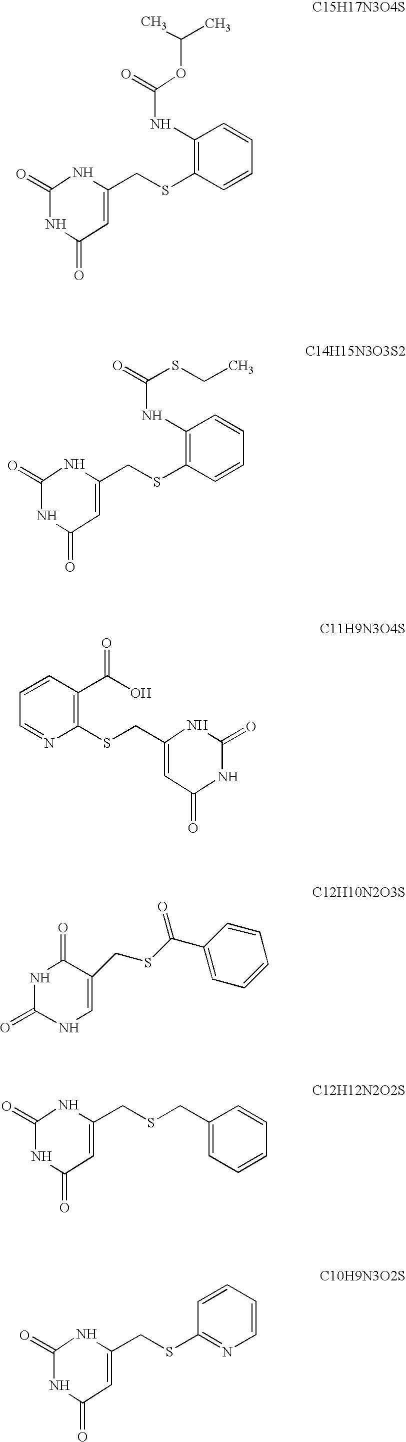

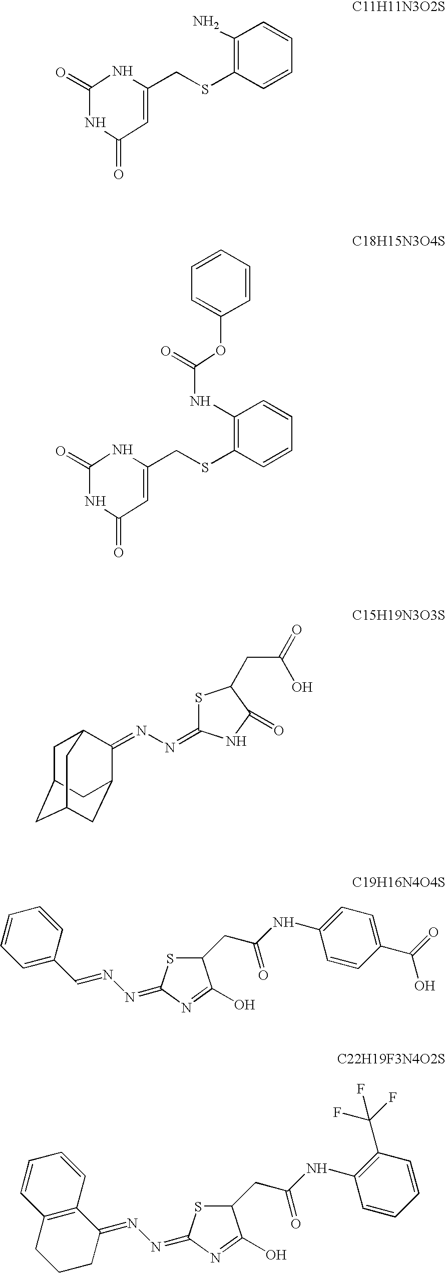

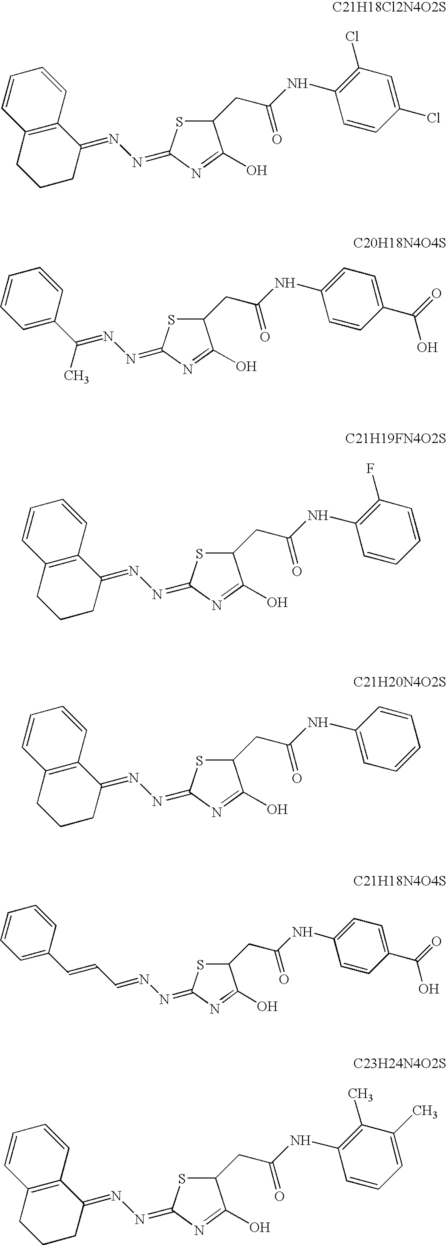

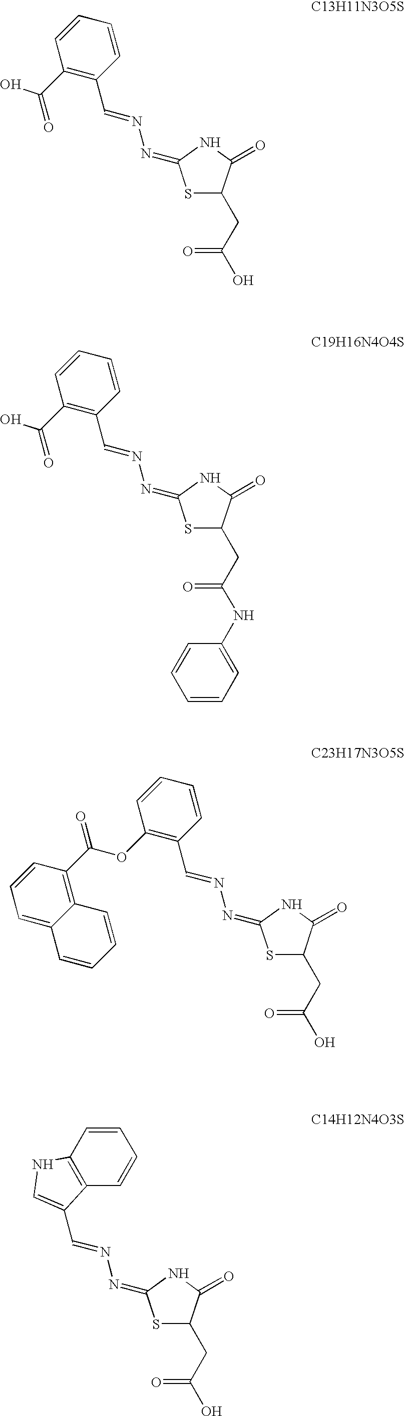

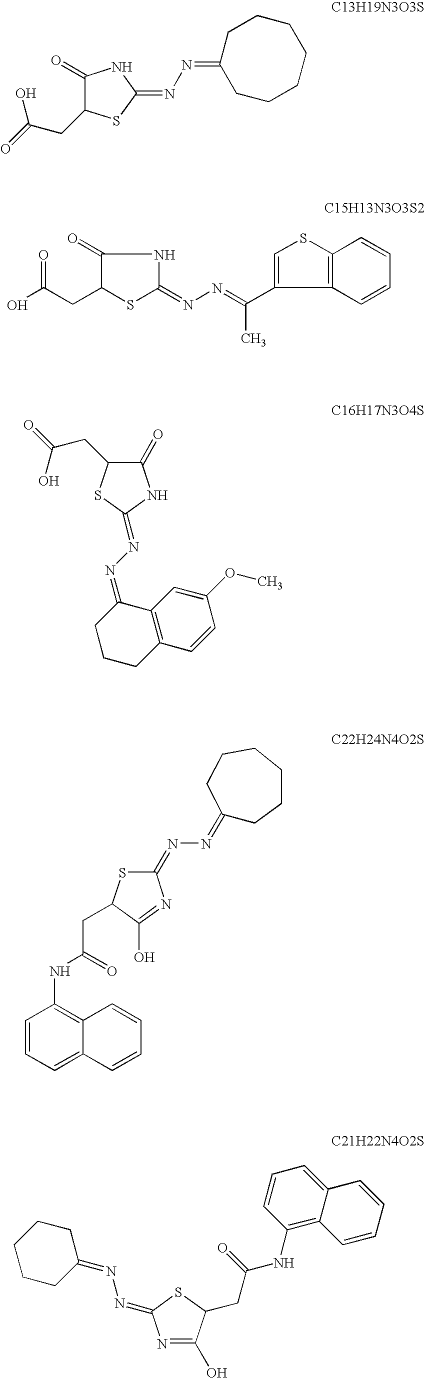

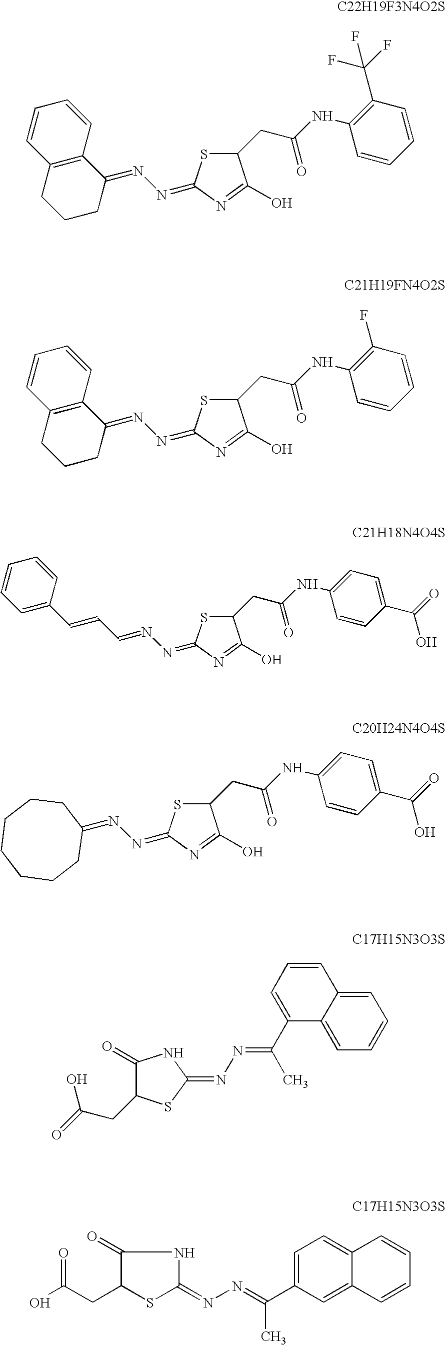

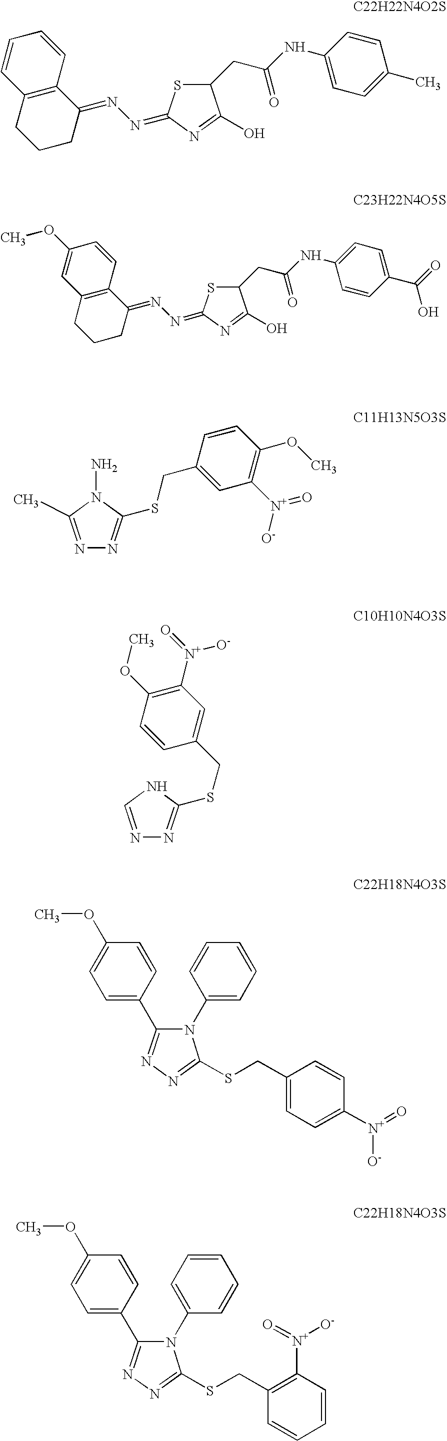

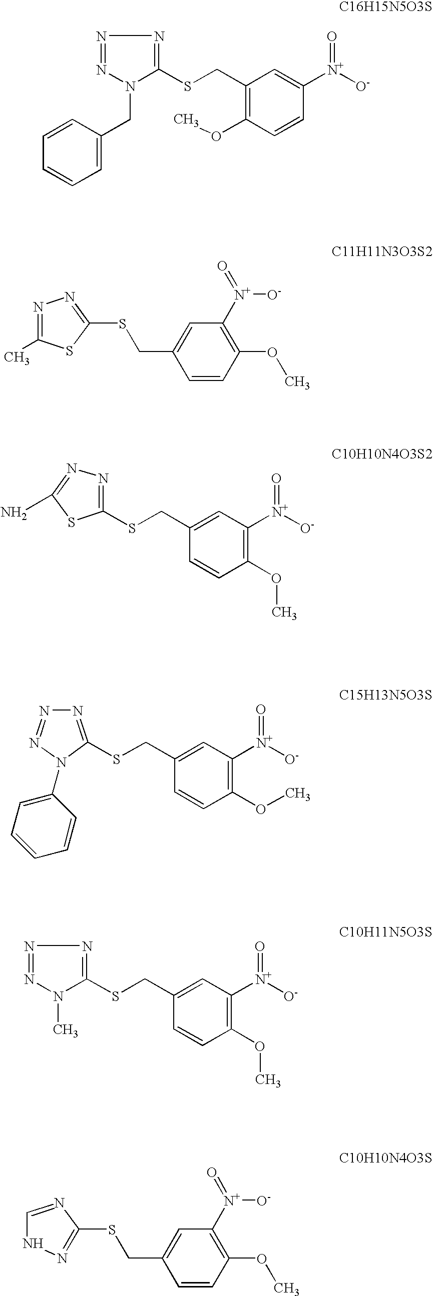

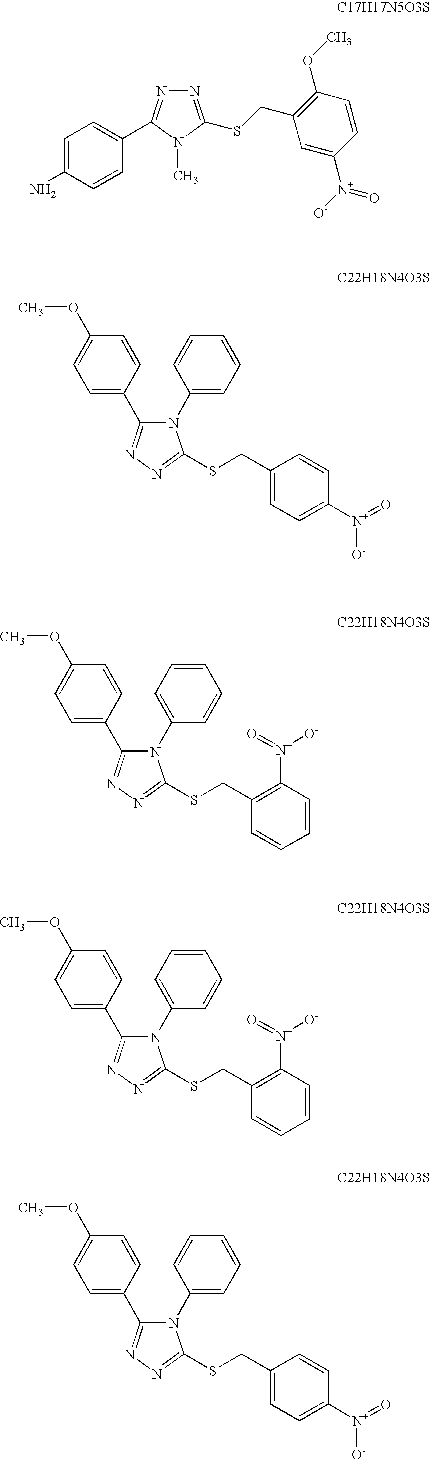

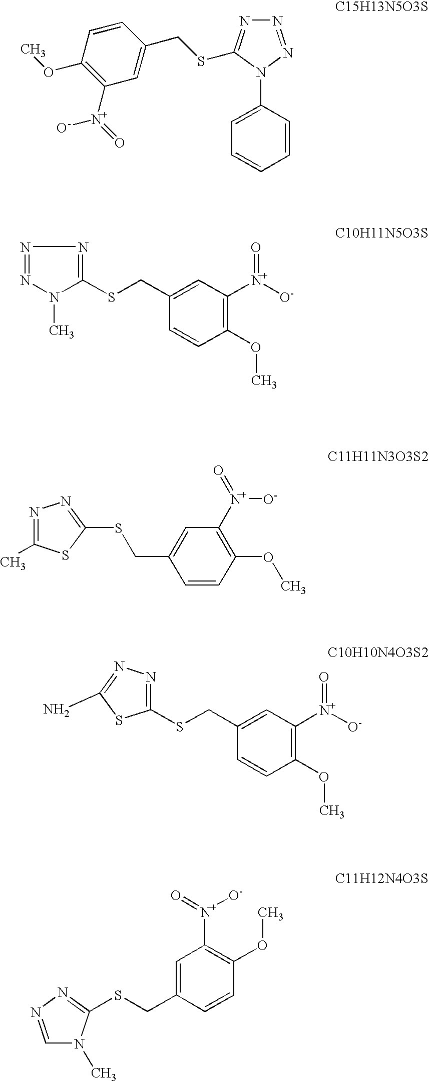

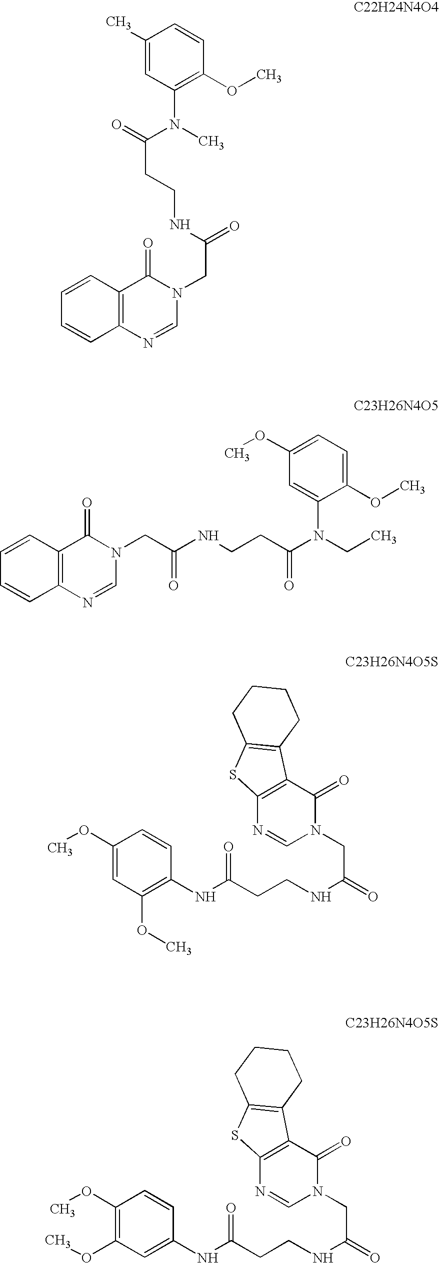

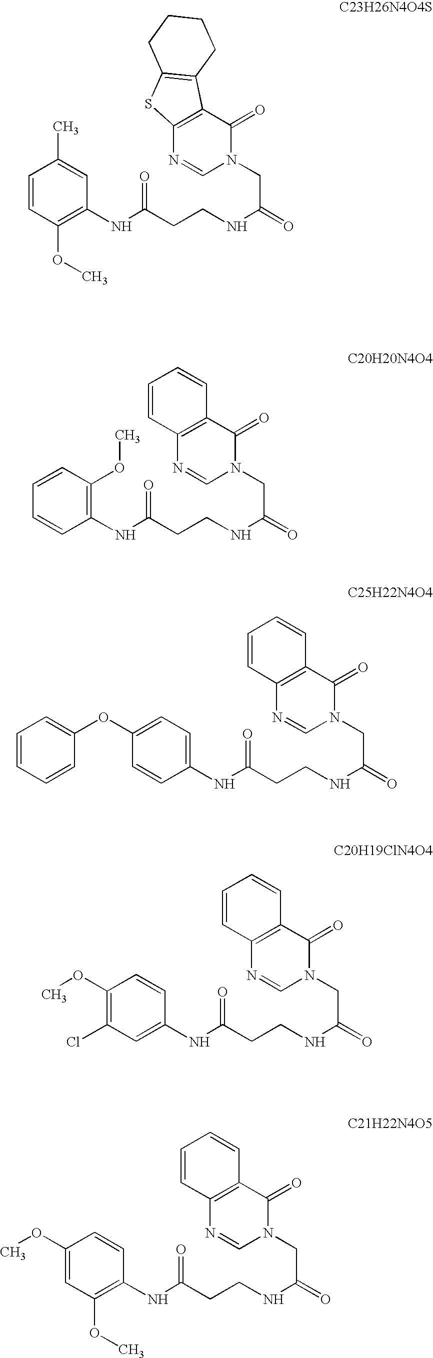

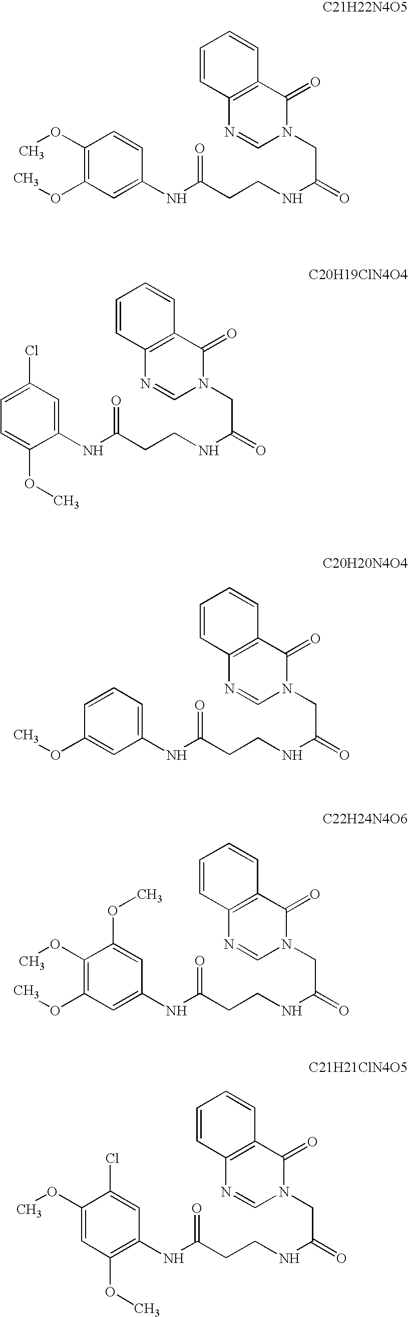

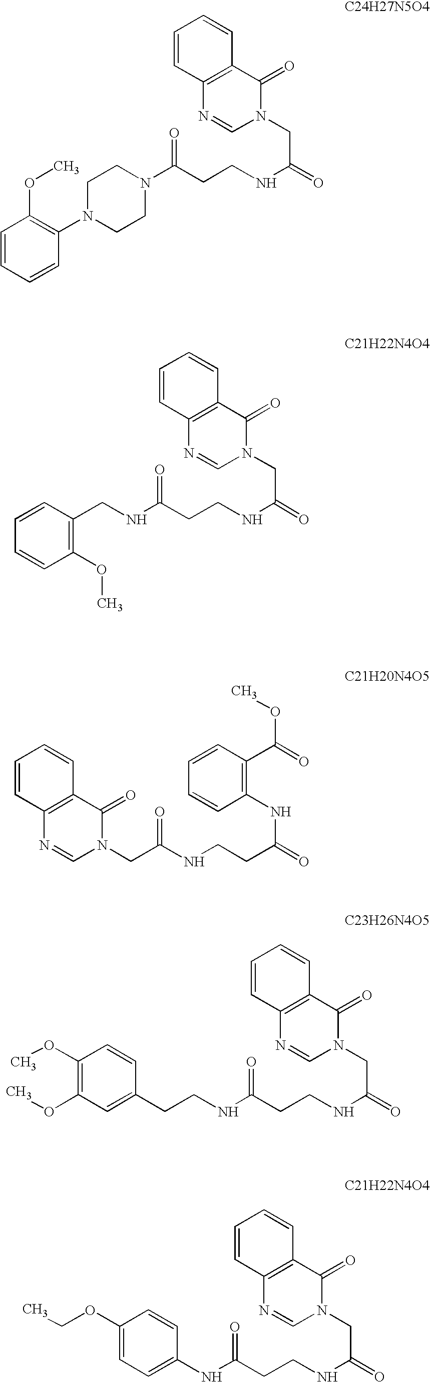

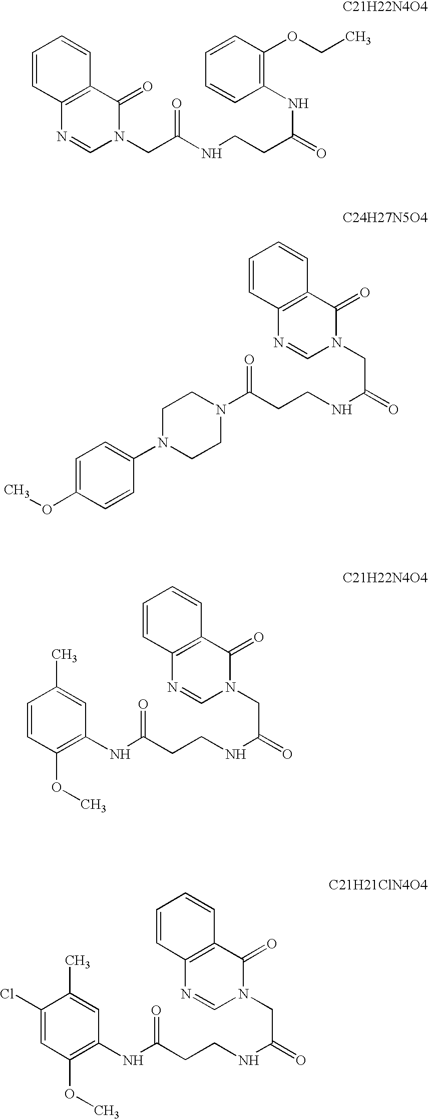

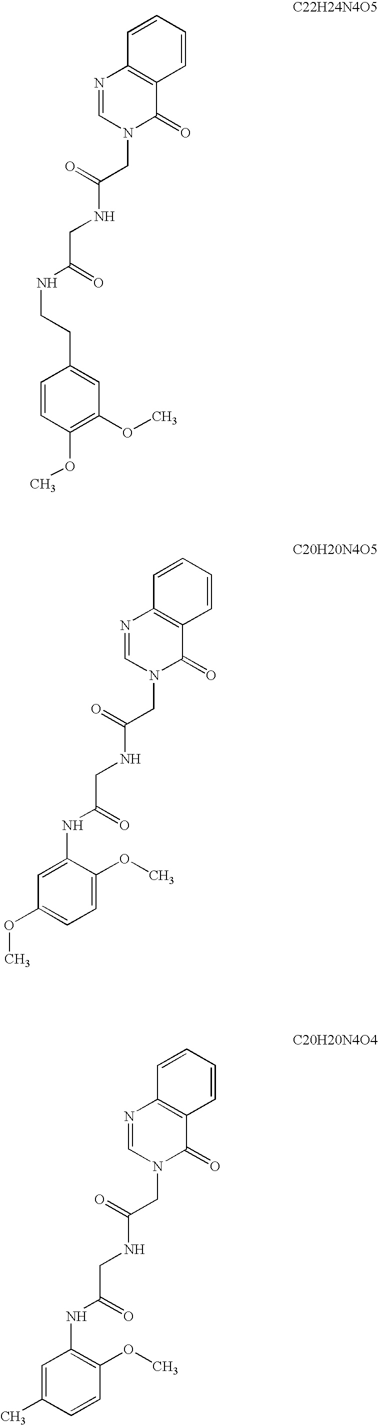

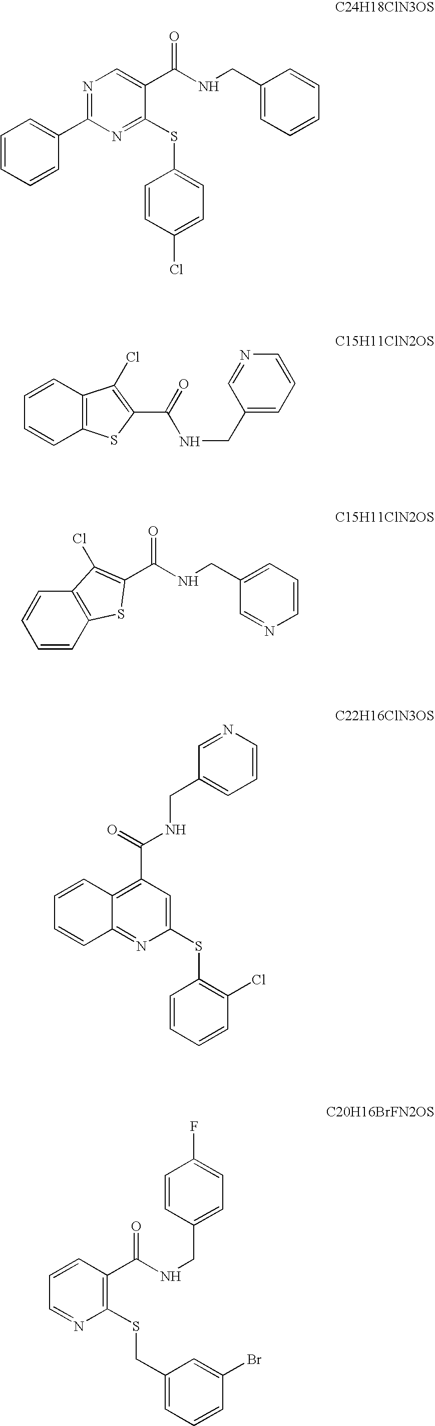

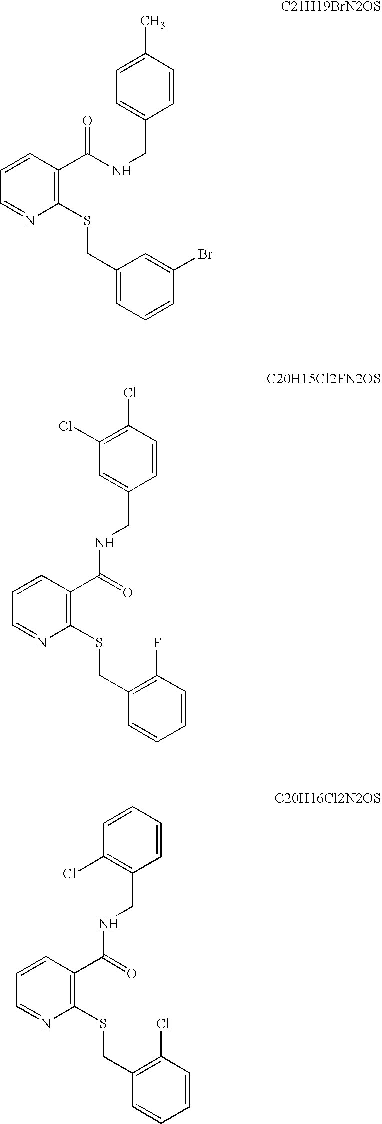

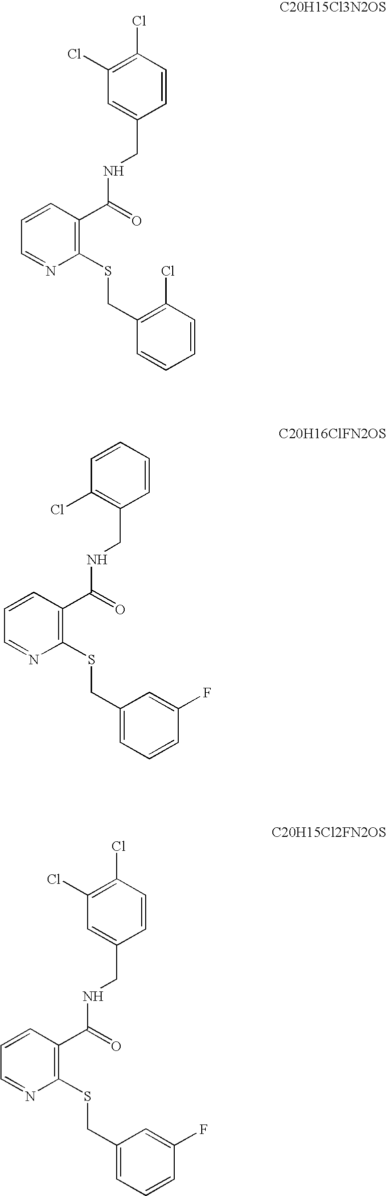

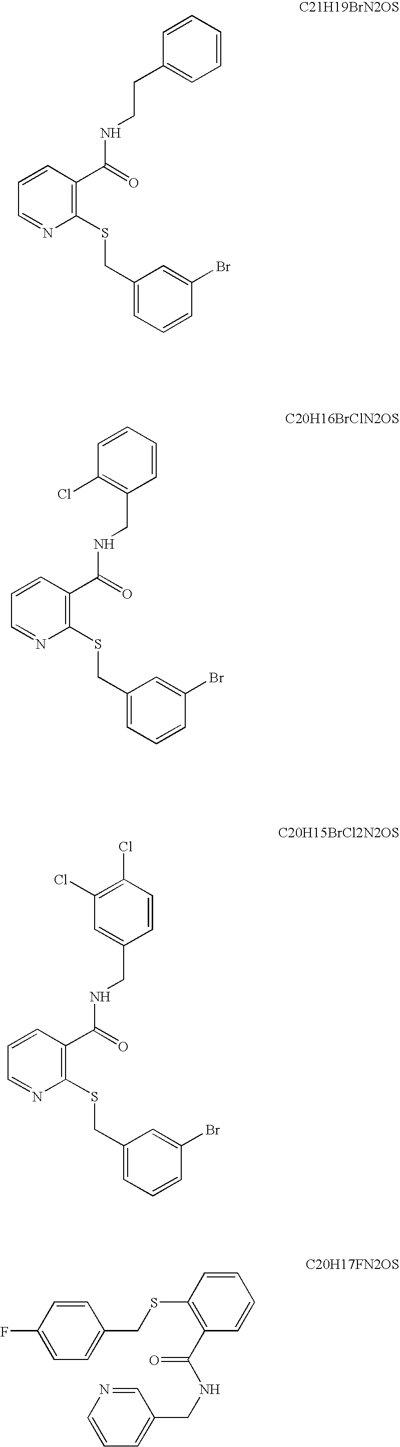

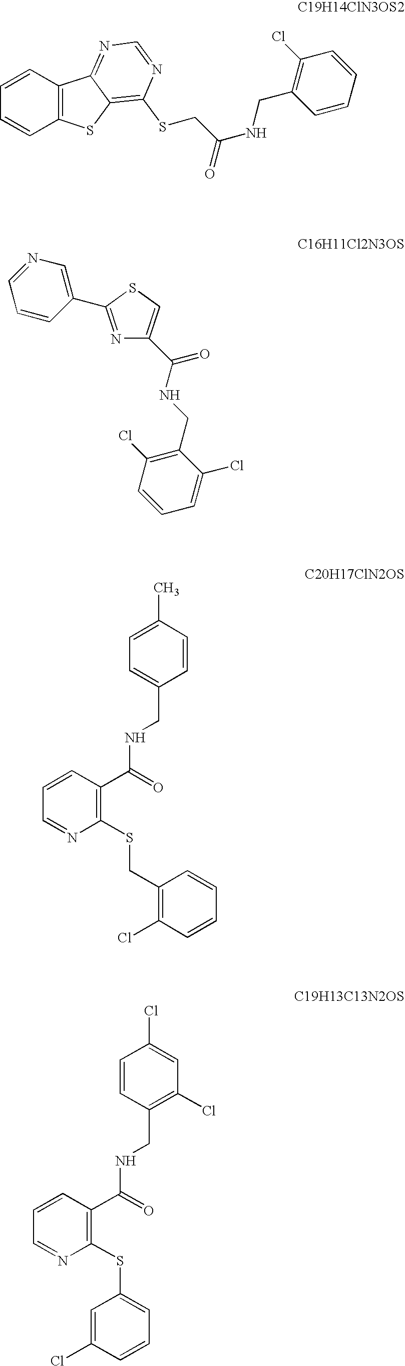

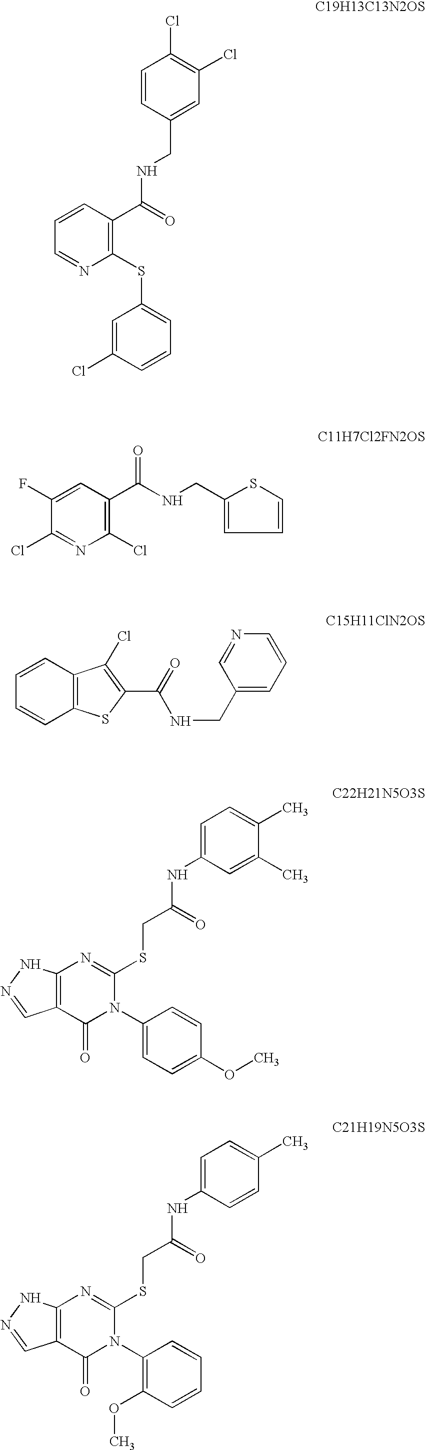

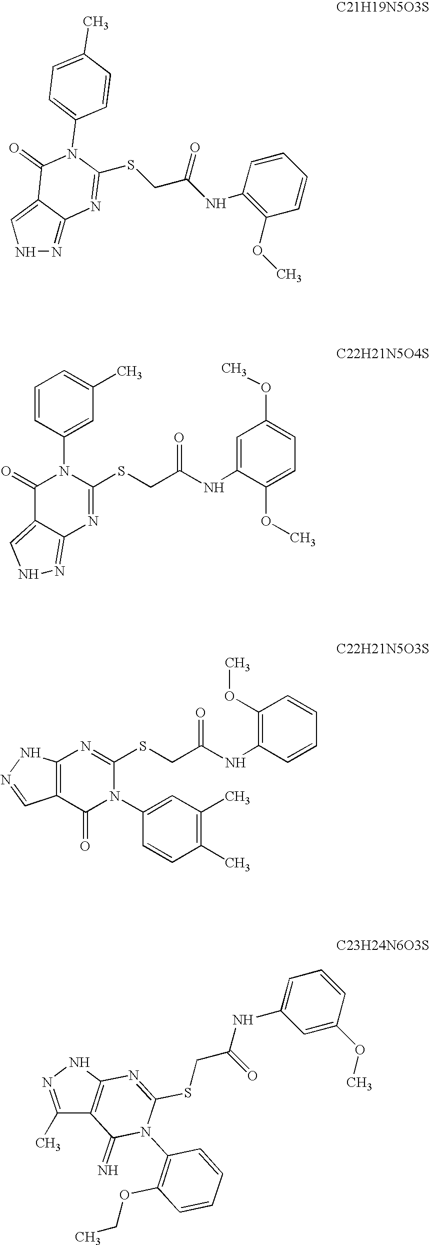

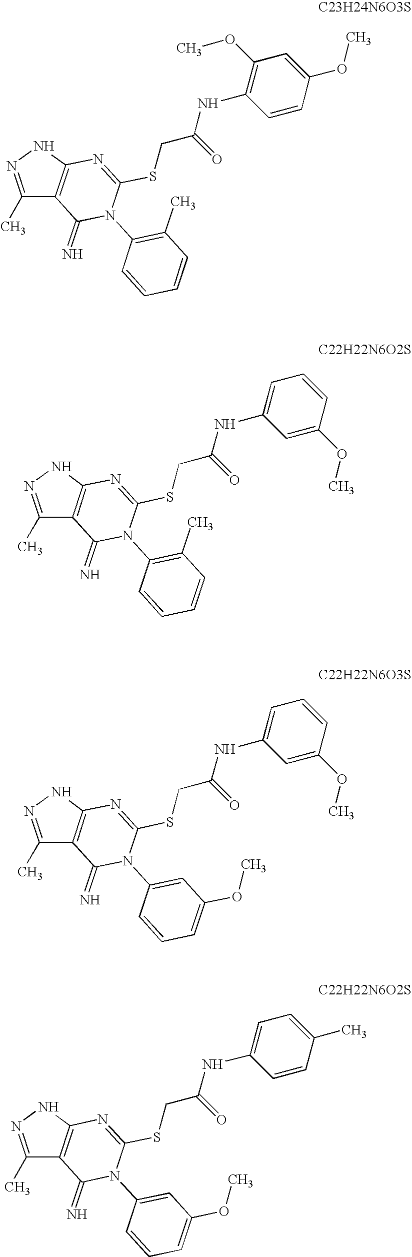

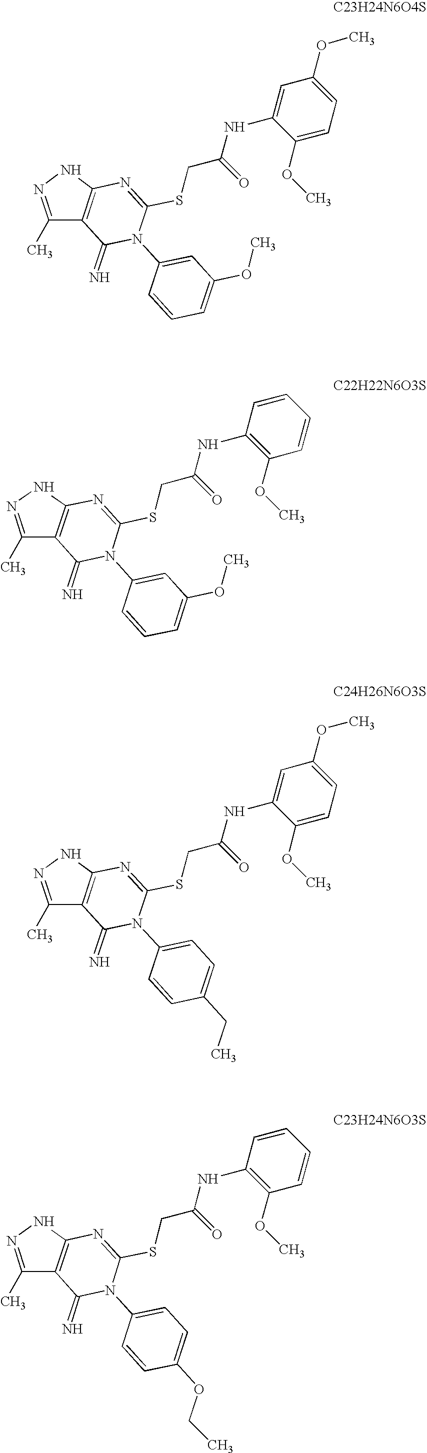

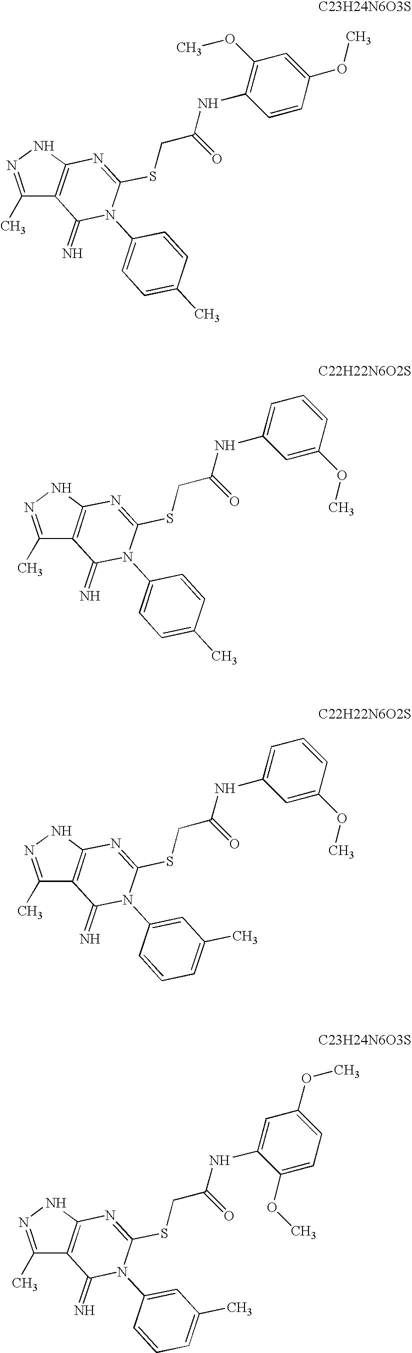

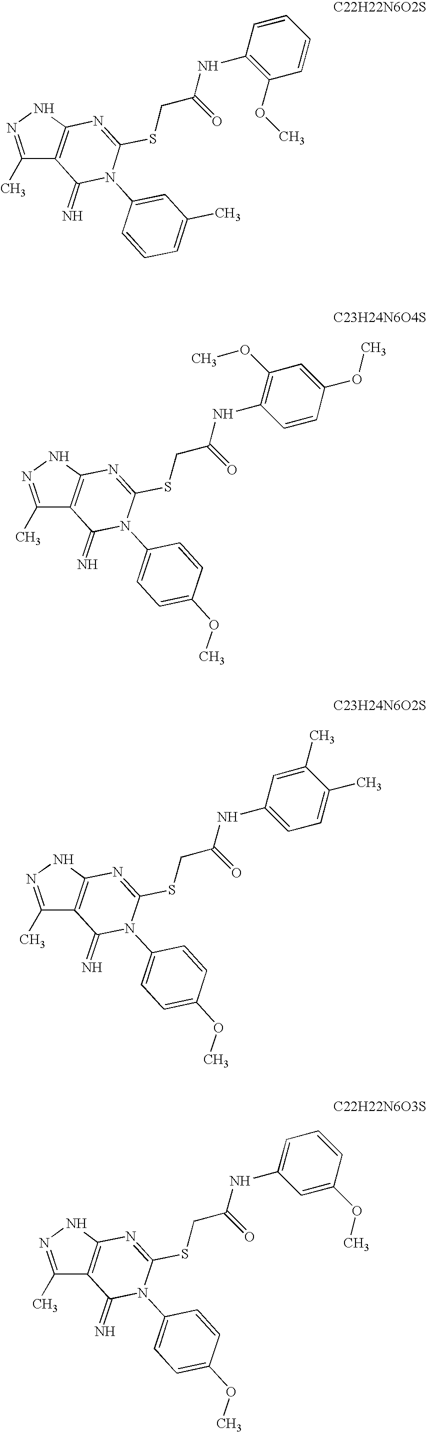

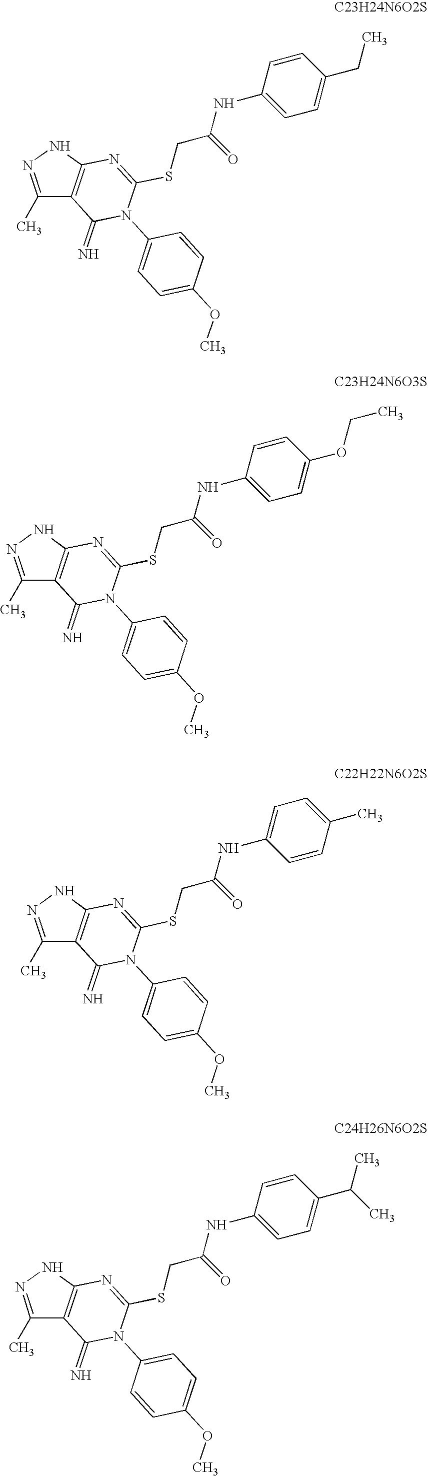

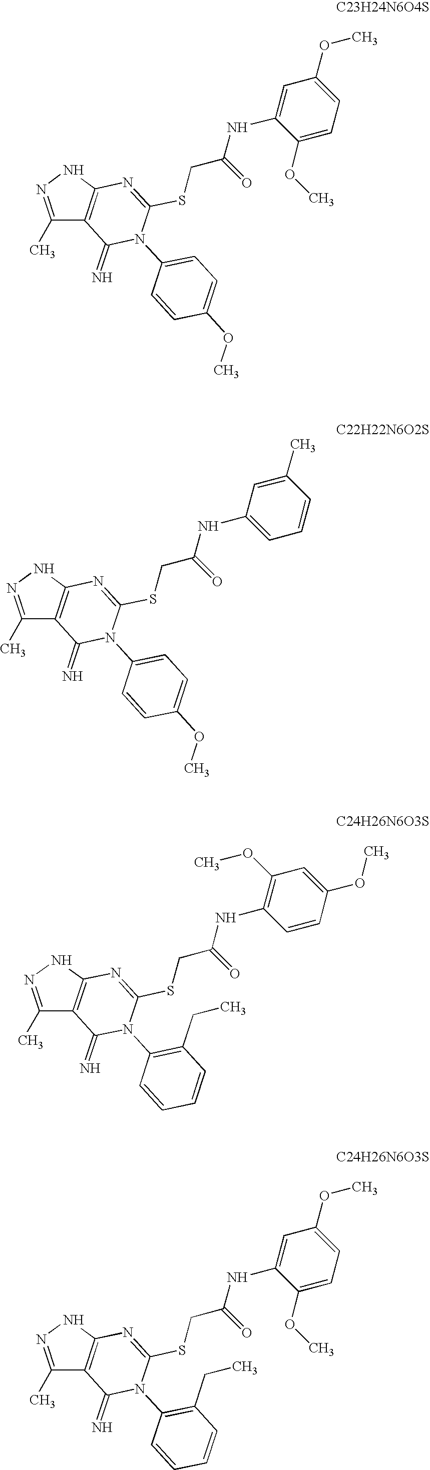

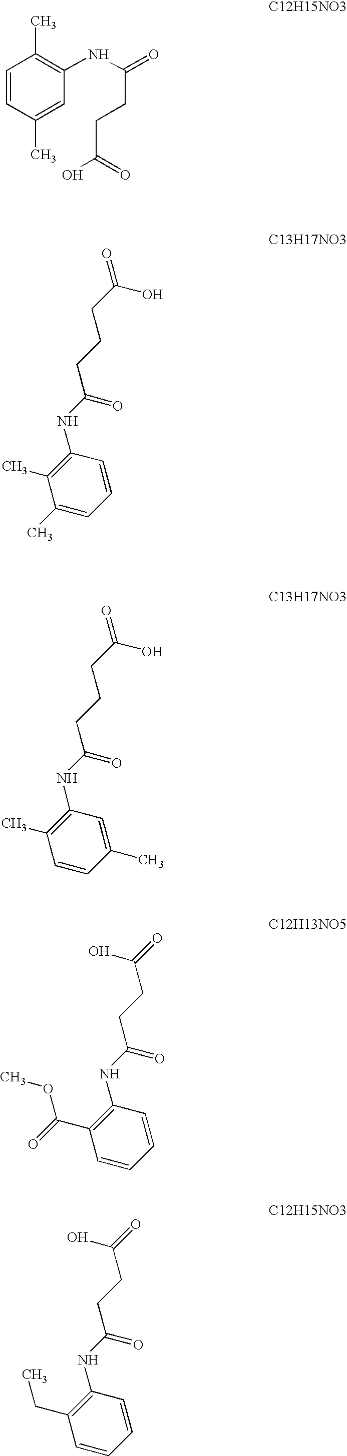

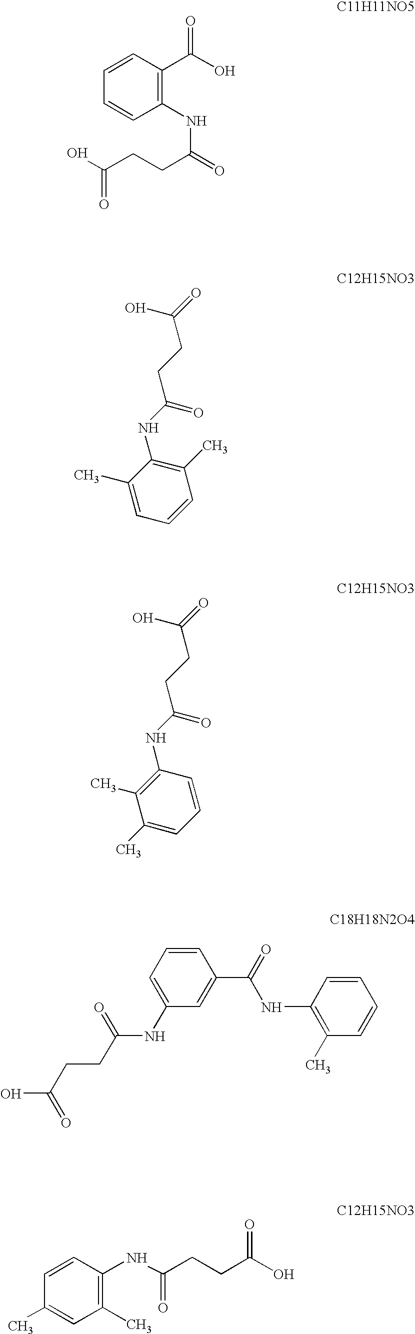

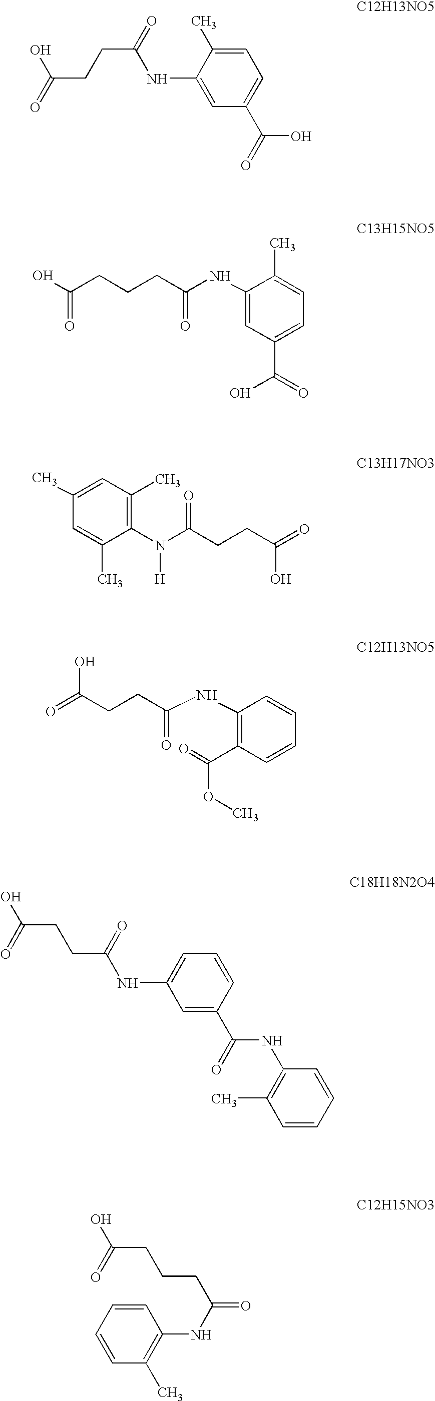

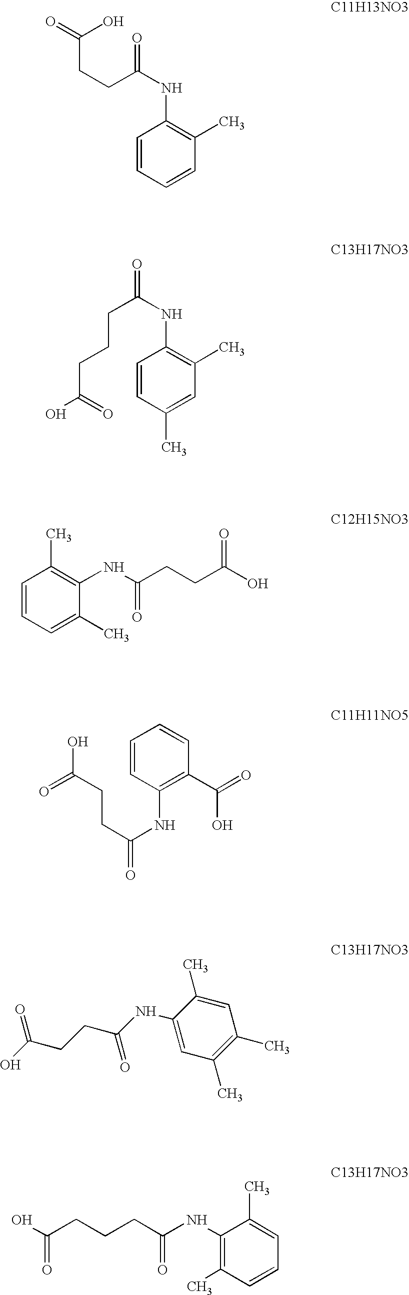

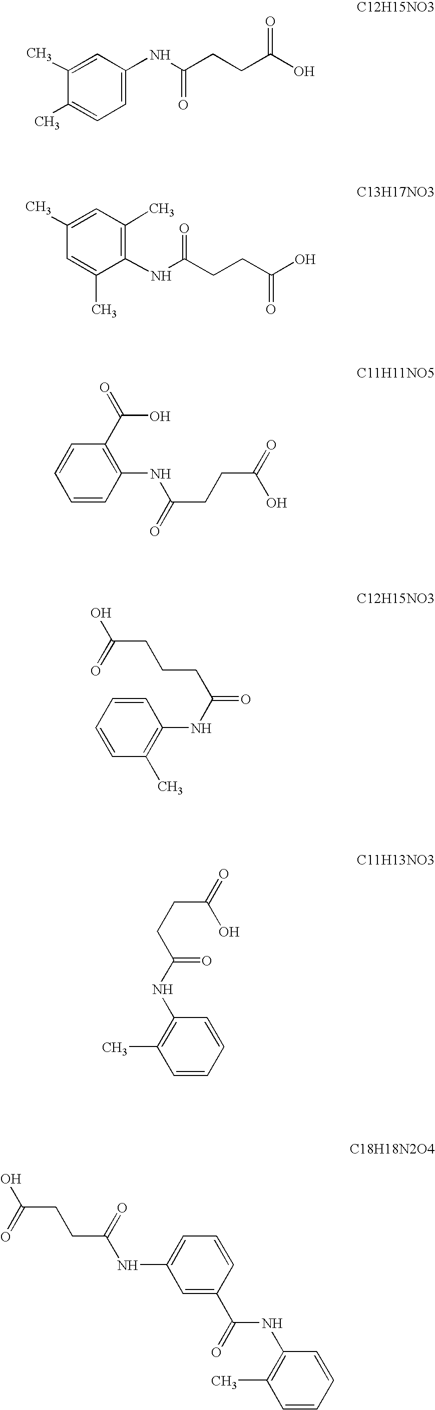

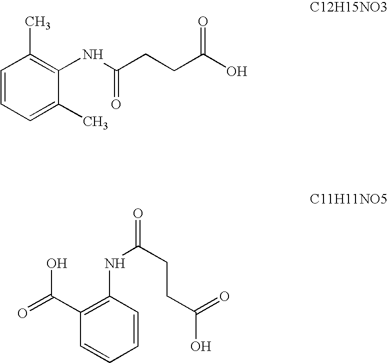

All compounds can be prepared fully conventionally, using known reaction chemistry, starting from known materials or materials conventionally preparable. [See, e.g., Houben-Weyl, Methoden der Organischen Chemie [Methods of Organic Chemistry], Georg-Thieme-Verlag, Stuttgart]. Most compounds of the invention are readily available from standard sources, such as chemical supply houses, or can be generated from commercially available compounds by routine modifications. All tested compounds were purchased from commercial vendors and all compounds specifically described in the application are known compounds.

Among the advantages of the compounds of the invention are that the molecules are not susceptible to enzymatic hydrolysis (as are certain peptide and protein modulators of protein tyrosine kinase activity), and that they exhibit good cell permeability characteristics.

Without wishing to be bound to any particular mechanism, this invention relates, e.g., to compounds that interact specifically with proteins, e.g., protein tyrosine kinases, which are involved in intracellular signaling pathways, in particular to compounds that interact with SH2 domains of such tyrosine kinases, and more particularly to compounds that interact with an SH2 domain of the p56^{lck} src family tyrosine kinase. Among other functions, the p56^{lck} protein is involved in signal transduction pathways involved in T cell antigen receptor activation signaling required for mounting an active immune response, and in aspects of cell proliferation, e.g., proliferation of neoplastic cells. It is proposed that compounds of the invention, by interacting with p56^{lck}, particularly with an SH2 domain thereof, modulate the kinase activity of the protein and/or modulate its ability to interact with a corresponding cellular binding protein, and thereby modulate immune responses, directly or indirectly, and neoplastic cell proliferation. Compounds of the invention can either enhance or inhibit signal transduction pathways, including downstream signal transduction processes in a signal transduction pathway, or they can be biphasic, either enhancing or inhibiting, depending on conditions. The effect of any given compound can be routinely determined by screening in one or more of the assays described herein or other fully conventional assays.

The non-catalytic domains of p56^{lck} kinase, e.g. the SH2 domain(s), mediate specific intramolecular and intermolecular interactions that are important for the regulation of p56^{lck} function; they exert both negative and positive effects on kinase activity. In general, the intramolecular interaction keeps p56^{lck} in an inactive state, and the intermolecular interactions facilitate p56^{lck} kinase action. For example, the SH2 domain can positively regulate p56^{lck} enzymatic activity by targeting p56^{lck} to specific cellular sites [ITAM (immunoreceptor tyrosine based activation motifs) phosphotyrosines containing peptides] where substrate phosphorylation is needed; and p56^{lck} that is bound to phosphtyrosine sites via its SH2 domain can exhibit higher enzymatic activity, thereby enhancing further phosphorylation of substrates. Without wishing to be bound to any particular mechanism as to how this is accomplished, it is proposed that the compounds which bind to the SH2 domain can either increase (activate, enhance, stimulate), decrease (suppress, inhibit, depress), or have no effect on, kinase activity and attendant cellular phosphorylation events (e.g., processes involved in intracellular signaling). p56^{lck} plays an important role in modulating immune responses. p56^{lck} is a T-cell specific kinase, the majority of which is associated with CD4 (in T_{H} cells) and CD8 (in cytotoxic T cells). The p56^{lck} kinase is responsible, e.g., for an early step in activating T cells —the phosphorylation of ITAM in CD3 chains—which in turn initiates multiple intracellular cascades of biochemical events leading to, e.g., actin polymerization, enhanced gene transcription, cellular proliferation and differentiation. p56^{lck} also plays an important role in a second important step in the activation of T cells—immunological synapse formation. The compounds of the invention can modulate the immune response by, e.g. modulating T-cell activation, or indirectly by modulating downstream processes of a signal transduction pathway. As used in this application, the term “modulate” means to change, e.g., to increase (activate, enhance, stimulate) or decrease (suppress, inhibit, depress) a reaction or an activity. Compounds of the invention can be said to modulate the binding of a p56^{lck} SH2 domain to a “corresponding cellular binding protein,” which term, as used herein, refers to any cellular binding protein whose binding to p56^{lck} is mediated by SH2 domains. Such corresponding cellular binding proteins include, e.g., CD3 chains, ZAP-70, p62, Lad, CD45, Sam68 or the like.

Many protein tyrosine kinases play a role in regulating cellular events, including gene activation and/or regulation, and thus, e.g., in cell proliferation. p56^{lck} is a proto-oncogene, which has been implicated in a number of pathological conditions that involve undesirable hyperproliferation of cells. For example, overexpression of constitutively active p56^{lck} has been observed in murine and human lymphomas, suggesting that p56^{lck}-mediated phosphorylation of cellular proteins stimulates lymphocyte proliferation. In addition, overexpression and activation of p56^{lck} appears to play an important role in the human lymphoid cell transformation-induced by Epstein-Barr virus and Herpesvirus Saimiri. Moreover, transgenic mice overexpressing wild type p56^{lck} and a constitutively active form of p56^{lck} in thymocytes develop thymoma, suggesting that even the overexpression of wild type p56^{lck} can transform cells under these conditions. Compounds of the invention, e.g. compounds which inhibit p56^{lck} activity, are useful for the treatment of conditions involving hyperproliferative cell growth, either in vitro (e.g., transformed cells) or in vivo. Conditions which can be treated or prevented by the compounds of the invention include, e.g., a variety of neoplasms, including benign or malignant tumors, a variety of hyperplasias, or the like. Compounds of the invention can achieve the inhibition and/or reversion of undesired hyperproliferative cell growth involved in such conditions.

As used herein, the term “hyperproliferative cell growth” refers to excess cell proliferation. The excess cell proliferation is relative to that occurring with the same type of cell in the general population and/or the same type of cell obtained from a patient at an earlier time. “Hyperproliferative cell disorders” refer to disorders where an excess cell proliferation of one or more subsets of cells in a multicellular organism occurs, resulting in harm (e.g., discomfort or decreased life expectancy) to the multicellular organism. The excess cell proliferation can be determined by reference to the general population and/or by reference to a particular patient (e.g., at an earlier point in the patient's life). Hyperproliferative cell disorders can occur in different types of animals and in humans, and produce different physical manifestations depending upon the affected cells. Hyperproliferative cell disorders include, e.g., cancers, blood vessel proliferative disorders, fibrotic disorders, and autoimmune disorders.

Activities and other properties of the compounds of the invention (and comparisons of those activities to those of art-recognized, comparison compounds) can be measured by any of a variety of conventional procedures.

A variety of in vitro assays can be used to measure biological and/or chemical properties of the compounds, and are conventional in the art. For example, in vitro binding studies can determine the affinity and the specificity of binding of the compounds, e.g., to a p56^{lck} SH2 domain. Assay Example 4 illustrates a method to determine K_{D} and IC₅₀ values, using tritiated compounds and purified, recombinant p56^{lck} SH2 domains. Similar assays can show that compounds bind selectively in vitro to a particular site, e.g., to the p56^{lck} SH2 domain, but not to other sites, e.g., Hck, Fyn, Src, Shc or ZAP-70 SH2 domains. Assay Example 5 illustrates an in vitro co-immunoprecipitation (IP) kinase assay. Again, similar assays can show the specificity of binding of the compounds. Assay Example 6 illustrates an assay to determine specificity of the binding.

Other conventional in vitro assays can measure the effect (e.g., inhibition or enhancement) of the compounds on biological activities associated with tyrosine protein kinases, e.g., p56^{lck}. p56^{lck} activities which are involved in immune responses include, e.g., the phosphorylation of, e.g., tyrosine in the ITAM consensus sequence present in certain molecules, e.g., CD3 chains; immunological synapse formation, e.g., with corresponding cellular binding proteins; or the like. Assay Example 1 illustrates an in vitro assay for Jurkat cell-activation-dependent phosphorylation, an activity that is correlated with T-cell activation. Assay Example 2 illustrates an in vitro assay for cell viability, which indicates if a compound is cytotoxic or cytostatic. Assay Example 3 illustrates an in vitro assay for IL-2 production, an activity Which is correlated with T-cell activation. Assay Example 7 illustrates a mixed lymphocyte culture assay.

A variety of in vivo assays can be used to demonstrate immunomodulatory properties of the compounds. Such in vivo assays, and appropriate animal models for disease conditions that can be treated with the compounds, are well-known to those of skill in the art. For example, animal models for rheumatoid arthritis are illustrated in Assay Example 8.

Assays to measure the effect of compounds (e.g., phosphotyrosine kinase inhibitors) on cell growth (proliferation) and cell transformation are conventional. A variety of typical assays are described, e.g., in Kelloff, G. J., et al., Cancer Epidemiol Biomarkers Prev., 1996. 5(8), p. 657-66; Wakeling, A. E., et al., Breast Cancer Res Treat, 1996, 38(1), 67-73; Yano, S., et al., Clin Cancer Res, 2000, 6(3), p. 957-65; Reedy, K. B., et al., Cancer Res, 1992, 52(13), p. 3636-41; Peterson, G. and S. Barnes, Prostate, 1993; 22(4), p. 335-45; Scholar, E. M. and M. L. Toews, Cancer Lett, 1994, 87(2); 159-62; Spinozzi, F., et al., Leuk Res, 1994, 18(6), p. 431-9; Kondapaka, B. S. and K. B. Reddy, Mol Cell Endocrinol, 1996, 117(1), p. 53-8; Moasser, M. M., et al., Cancer Res, 1999, 59(24), p. 6145-52; Li, Y., M. Bhuivan & F. H. Sarkar, Int J Oncol, 1999, 15(3), p. 525-33; Baguley, B. C., et al. Eur J Cancer, 1998, 34(7), p. 1086-90; and bhatia, R., H. A. Munthe, and C. M. Verfaillie, Leukemia, 1998, 12(11), p. 1708-17.

Variations of the assays described herein, as well as other conventional assays, are well known in the art. Such assays can, of course, be adapted to a high throughput format, using conventional procedures.

The compounds of the invention are effective for binding to, e.g., p56^{lck} SH2 domains, and for modulating the activity of, e.g., p56^{lck} in animals, e.g., mammals, such as mouse, rat, rabbit, pets, (e.g., mammals, birds, reptiles, fish, amphibians), domestic (e.g., farm) animals, and primates, especially humans. The inventive compounds exhibit, e.g., immunomodulatory activity and/or antineoplastic activity, and are effective in treating diseases in which, e.g., aberrant regulation or activity of tyrosine kinase (e.g., p56^{lck}) and/or intracellular signaling responses are involved. For example, compounds which stimulate immune responses (immunostimulants) are useful for treating or preventing naturally occurring immunosuppression or immunosuppression from a variety of conditions and diseases. Compounds which depress immune responses (immunosuppressants) are useful for treating or preventing, e.g., autoimmune diseases which are characterized by inflammatory phenomena and destruction of tissues caused by the production, by the immune system, of the body's own antibodies, or for suppressing rejection during, e.g., tissue or organ transplantation. Compounds which inhibit cell proliferation are useful for treating conditions characterized by cell hyperproliferation, e.g., as antineoplastic agents. Compounds of the invention are also useful as research tools, e.g., to investigate cell signaling.

In accordance with a preferred embodiment, the present invention includes methods of treating patients suffering from depressed immune systems resulting from, e.g., chemotherapy treatment, radiation treatment, radiation sickness, or HIV/AIDs; conditions associated with primary B-cell deficiency (such as, e.g., Bruton's congenital a-γ-globulinemia or common variable immunodeficiency) or primary T-cell deficiency (such as, e.g., the DiGeorge and Nezelof syndromes, ataxia telangiectasia or Wiskott-Aldrich syndrome); severe combined immunodeficiency (SCID), etc.; with an immunostimulant of the invention. The immunostimulants can also be used for vaccines (e.g., anti-bacterial, anti-fungal, anti-viral or anti-protozoiasis), particularly for patients having immunocompromised states; or for anti-neoplastic vaccines.

In another preferred embodiment, the invention includes methods of treating patients suffering from autoimmune disorders, such as, e.g., rheumatoid arthritis, glomerulonephritis, Hashimoto's thyroiditis, multiple sclerosis, T cell leukemia, systemic lupus erythematosus, myasthenia gravis, autoimmune hemolytic anemia, autoimmune thrombocytopenic purpura, type 1 diabetes, Chrohn's disease, Grave's disease, celiac disease, or the like, with an immunosuppressant of the invention. Immunosuppressants of the invention are also useful for treating tissue or organ transplant rejection, e.g., hyper-acute or chronic graft-vs-host disease, allograft or xenograft rejection, etc.

As mentioned, the compounds of the invention also inhibit hyperproliferation of cells, e.g., they can exhibit anti-neoplastic activity. As a result, the inventive compounds are useful in the treatment of a variety of conditions, e.g. cancers involving T cells and B cells. Among the types of cancer which can be treated with compounds of the invention are e.g., leukemias, lymphomas, ovarian cancer and breast cancer.

Compounds of the invention can be attached to an agent that, e.g., targets certain tumors, such as an antibody which is specific for a tumor-specific antigen. In this manner, compounds of the invention can be transported to a target cell in which they then can act. The compounds can be further attached to a conventional cytotoxic agent (such as a toxin or radioactivity). When the inventive molecule binds to its target, e.g., p56^{lck}, it not only will inhibit the enzymatic activity, but will also destroy the target, and/or the cell in which the target resides, by means of the toxin.

The preferred aspects include pharmaceutical compositions comprising a compound of this invention and a pharmaceutically acceptable carrier and, optionally, another active agent as discussed below; a method of inhibiting or stimulating a p56^{lck} kinase, e.g., as determined by a conventional assay or one described herein, either in vitro or in vivo (in an animal, e.g., in an animal model, or in a mammal or in a human); a method of modulating an immune response, e.g., enhancing or inhibiting an immune reaction; a method of treating a disease state, e.g., an autoimmune disease, a neoplasm, etc.; a method of treating a disease state modulated by p56^{lck} kinase activity, in a mammal, e.g., a human, including those disease conditions mentioned herein.

The present invention also relates to useful forms of the compounds as disclosed herein, such as pharmaceutically acceptable salts and prodrugs of all the compounds of the present invention. Pharmaceutically acceptable salts include those obtained by reacting the main compound, functioning as a base, with an inorganic or organic acid to form a salt, for example, salts of hydrochloric acid, sulfuric acid, phosphoric acid, methane sulfuric acid, camphor sulfonic acid, oxalic acid, maleic acid, succinic acid and citric acid.

Pharmaceutically acceptable salts also include those in which the main compound functions as an acid and is reacted with an appropriate base to form, e.g., sodium, potassium, calcium, magnesium, ammonium, and chlorine salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts are prepared by reacting the compounds of the invention with the appropriate base via a variety of known methods.

The following are further examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates,.hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropiionates, picrates, pivalates, propionates, succinates, tartrates, thiocyannates, tosylates, mesylates and undecanoates.

Preferably, the salts formed are pharmaceutically acceptable for administration to mammals. However, pharmaceutically unacceptable salts of the compounds are suitable as intermediates, for example, for isolating the compound as a salt and then converting the salt back to the free base compound by treatment with an alkaline reagent. The free base can then, if desired, be converted to a pharmaceutically acceptable acid addition salt.

The compounds of the invention can be administered alone or as an active ingredient of a formulation. Thus, the present invention also includes pharmaceutical compositions of compounds of formulas I to XVII, preferably of formulae I to IX, or a salt thereof, containing, for example, one or more pharmaceutically acceptable carriers.

Numerous standard references are available that describe procedures for preparing various formulations suitable for administering the compounds according to the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Isol, editor), 1553-1593 (current edition).

In view of their high degree of selective p56^{lck} kinase inhibition or stimulation, the compounds of the present invention can be administered to anyone requiring p56^{lck} kinase inhibition or stimulation. Administration may be accomplished according to patient needs, for example, orally, nasally, parenterally (subcutaneously, intravenously, intramuscularly, intrastemally, and by infusion) by inhalation, rectally, vaginally, topically and by ocular administration. Injection can be, e.g., intramuscular, intraperitoneal, intravenous, etc.

Various solid oral dosage forms can be used for administering compounds of the invention including such solid forms as tablets, gelcaps, capsules, caplets, granules, lozenges and bulk powders. The compounds of the present invention can be administered alone or combined with various pharmaceutically acceptable carriers, diluents (such as sucrose, mannitol, lactose, starches) and excipients known in the art, including but not limited to suspending agents, solubilizers, buffering agents, binders, disintegrants, preservatives, colorants, flavorants, lubricants and the like. Time-release capsules, tablets and gels are also advantageous in administering the compounds of the present invention.

Various liquid oral dosage forms can also be used for administering compounds of the inventions, including aqueous- and non-aqueous solutions, emulsions, suspensions, syrups, and elixirs. Such dosage forms can also contain suitable inert diluents known in the art such as water and suitable excipients known in the art such as preservatives, wetting agents, sweeteners, flavorants, as well as agents for emulsifying and/or suspending the compounds of the invention. The compounds of the present invention may be injected, for example, intravenously, in the form of an isotonic sterile solution. Other preparations are also possible.

Suppositories for rectal administration of the compounds of the present invention can be prepared by mixing the compound with a suitable excipient such as cocoa butter, salicylates and polyethylene glycols. Formulations for vaginal administration can be in the form of a pessary, tampon, cream, gel, paste, foam, or spray formula containing, in addition to the active ingredient, such suitable carriers as are known in the art.

For topical administration the pharmaceutical composition can be in the form of creams, ointments, liniments, lotions, emulsions, suspensions, gels, solutions, pastes, powders, sprays, and drops suitable for administration to the skin, eye, ear or nose. Topical administration may also involve transdermal administration via means such as transdermal patches.

Aerosol formulations suitable for administering via inhalation also can be made. For example, for treatment of disorders of the respiratory tract, the compounds according to the invention can be administered by inhalation in the form of a powder (e.g., micronized) or in the form of atomized solutions or suspensions. The aerosol formulation can be placed into a pressurized acceptable propellant.

The compounds can be administered as the sole active agent or in combination with other pharmaceutical agents, such as other agents which inhibit or stimulate tyrosine kinases, signal transduction processes, cell proliferation and/or immune responses. Inhibitory agents include, e.g., cyclosporine, FK506, rapamycin, leflunomide, butenamindes, corticosteroids, atomeric acid, dipeptide derivative, tyrphostin, Doxorubicin or the like. In such combinations, each active ingredient can be administered either in accordance with its usual dosage range or a dose below its usual dosage range.

The dosages of the compounds of the present invention depend upon a variety of factors including the particular syndrome to be treated, the severity of the symptoms, the age, sex and physical condition of the patient, the route of administration, the frequency of the dosage interval, the particular compound utilized, the efficacy, toxicology profile, pharmacokinetic profile of the compound, and the presence of any deleterious side-effects, among other considerations.

By “effective dose” or “therapeutically effective dose” is meant herein, in reference to the treatment of a cancer, an amount sufficient to bring about one or more of the following results: reduce the size of the cancer; inhibit the metastasis of the cancer; inhibit the growth of the cancer, preferably stop cancer growth; relieve discomfort due to the cancer; and prolong the life of a patient inflicted with the cancer.

A “therapeutically effective amount,” in reference to the treatment of a hyper-proliferative cell disorder other than a cancer refers to an amount sufficient to bring about one or more of the following results: inhibit the growth of cells causing the disorder, preferably stopping the cell growth; relieve discomfort due to the disorder; and prolong the life of a patient suffering from the disorder.

A “therapeutically effective amount”, in reference to treatment of an autoimmune disorder refers to an amount sufficient to bring about one or more of the following results: inhibit or ameliorate the symptoms of the disease; inhibit progressive degeneration of cells involved in the disorder; relieve discomfort due to the disorder; and prolong the life of a patient suffering from the disorder.

A “therapeutically effective amount”, in reference to treatment of a patient undergoing tissue or organ transplantation refers to an amount sufficient to bring about one or more of the following results: inhibit or prevent rejection of the transplanted material; relieve discomfort resulting from rejection of the transplant; and prolong the life of a patient receiving a transplant.

A “therapeutically effective amount,” in reference to treatment of an immunosuppressive patient refers to an amount sufficient to bring about one or more of the following results: increase the number of T cells or number of activated T cells; reduce the immunosuppressed state of the patient; relieve discomfort due to the disorder; and prolong the life of a patient suffering from the disorder.

The compounds of the invention are administered at dosage levels and in a manner customary for p56^{lck} kinase inhibitors or stimulators, or other analogous drugs, such as those mentioned above. For example, cyclosporine is administered (for transplants) at about 7.95±2.81 mg/kg/day (see PDR(Physician's Desk Reference)); FK506 is administered (for transplants) at about 0.15-0.30 mg/kg/day (see PDR); and rapamycin is administered (for transplants) at about 2-6 mg/day, e.g., about 0.024 mg/kg/day for an 81 kg adult (see Thomas A. Stargy Transplantation Institute web site). See also, e.g., disclosures in U.S. Pat. Nos. 5,688,824, 5,914,343, 5,217,999, 6,133,301 and publications cited therein.

For example, compounds of formulae I to XVII, preferably of formulae I to IX, or a salt thereof, can be administered, in single or multiple doses, at a dosage level of, for example, 1 μg/kg to 500 mg/kg of body weight of patient/day, preferable between about 100 μg/kg/day and 25 mg/kg/day. Dosages can be adjusted so as to generate an immunostimulatory or immunosuppressive effect, as desired. A lower dosage (immunostimulatory) can be between about 1 μg /kg/day and 750 μg /kg/day, preferably between about 10 μg /kg/day and 500 mg/kg/day. A higher dosage (immunosuppressive) can be between about 1 mg/kg/day and 750 mg/kg/day, preferably between about 10 mg/kg/day and 450 mg/kg/day.

In carrying out the procedures of the present invention it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

In the foregoing and in the following examples, all temperatures are set forth in degrees Celsius; and, unless otherwise indicated, all parts and percentages are by weight.

### EXAMPLES

Virtual screening techniques resulted in the identification of several sets of compounds that are likely to target the pY+3 binding site and are likely potent inhibitors of the Lck SH2 domain. Compounds for biological assay were selected from the sets. Selecting compounds from the sets was based on several criteria, such as adequate solubility (ClogP values ≦5), molecular weight (≦500 Dalton), the number of the hydrogen bond donors and acceptors (≦10) and chemical stability.

In vitro Assay for the Inhibition of Binding of the CD3ζ ITAM2 Peptide to the SH2 Domain of p56 Lck. The peptide H-CAEApYSEIG(Nle)-OH was synthesized (Anaspec, San Jose, Calif.) and bound to agarose beads (Sulfolink Coupling kit, PIERCE, Rockford, Ill.). Inhibition of binding was assayed by affinity precipitation of p56Lck in cell lysates as follows.

a. Cell Culture and Lysis.

Jurkat cells were grown in RPMI-1640 medium containing glutamine (RPMI, BioSource, Camarillo, Calif.) with 10% supplemented calf serum (HyClone, Logan, Utah). Confluent cells were harvested, washed 3 times in chilled, sterile phosphate buffered saline (PBS, BioSource), and were resuspended (5×10⁵ /ml) in lysis buffer containing 1% Triton X-100 (Fisher), 1 mM EDTA (Fisher), 0.2 mM sodium orthovanadate, 500 μM AEBSF, 150 nM aprotinin, 1 μM E-64, 0.5 mM EDTA, and 1 μM leupeptin (Protease Cocktail Set I, Calbiochem, San Diego, Calif.). The suspension was sonicated on ice at 60 W power (VibraCell, Sonics and Materials, Danbury, Conn.) for 6×5 second bursts with a 1 minute cooling period between bursts. The resulting lysate was then centrifuged at 20,000 x g (RC-5B, Sorvall, Newtown, Conn.) at 4° C., and the supernatent was used in the assay.

b. Addition of Putative Inhibitors.

Putative inhibitors to be tested were freshly dissolved in dimethyl sulfoxide (DMSO, cell culture grade, Sigrna) at a concentration of 10 mM before the assay and 10 μl of this was added to 1 ml of cell lysate in 1.5 ml tubes (Sarstedt, Newton, North Carolina). The tubes were placed on an end-over-end rotator (Model C400110, Barnstead/Thermolyne, Dubuque, Iowa) at 4° C. and allowed to rotate for 30 minutes. 20 μl of a 50% slurry of the ITAM-conjugated agarose beads (in PBS; EM Science, Gibbstown, New Jersey) was then added to each tube, and tubes were rotated overnight.

c. Western Blotting.

The beads were washed 3 times in 800 μl lysis buffer. Excess lysis buffer was aspirated off, and 10 ml sample loading buffer containing 3% β-mercaptoethanol (Fisher) was added to the beads and, after brief vortexing and soiication, tubes were immersed in a boiling water bath for 2 minutes. They were then centrifuged and the supernatent was loaded onto 8% Tris-HCl gels (GeneMate, ISC Bioexpress, Kaysville, Utah) and run at 100 V for 1.5 hours in a Protean III Mini Gel Unit (Bio-Rad, Hercules, Calif.). The gels were transferred onto polyvinylidene fluoride membrane (Immobilon-P, Millipore, Bedford, Mass.) in a Mini Trans-Blot Electrophoretic Transfer Cell ,(Bio-Rad). The membrane was blocked with PBS (EM Science) solution containing 5% powdered milk (Nestle, Solon, Ohio) for 1 hour; exposed to primary antibody (anti-lck, rabbit polyclonal IgG, Upstate Biotechnologies, Lake Placid, N.Y.) at 1:5000 dilution in 1% w/v powdered milk (Nestle, Solon, Ohio) in PBST [PBS containing 0.5% v/v Tween 20 (Sigma, St. Louis, Mo.)] and washed 3 times for 5 minutes each in PBST. The membrane was then exposed to secondary antibody (peroxidase-labelled goat anti-rabbit IgG (KPL, Gaithersburg, Md.) at 1:5000 dilution in 1% milk-PB ST and washed 3 times for 15 minutes each in PBST. Immunoblots were visualized with SuperSignal West Femto Maximum Sensitivity Substrate (PIERCE) in the EpiChemi³ Darkroom equipped with a 12 lit cooled CCD camera (UVP, Upland, Calif.).

Physical Characterization of Active Compounds.

All the compounds showing activity in the in vitro assay were subjected to physical characterization. Molecular weights of the compounds were confirmed by mass spectrometry. Briefly, the compounds were dissolved in methanol with 0.1% acetic acid (both from Fisher Scientific, Pittsburgh, Pa.) and injected on a LCQ instrument (Finnigan Mat, San Jose, Calif.) and spectra were acquired using Thermo Finnigan software. The purity of the compounds was confirmed by thin layer chromatography. Briefly, compounds were dissolved in 5% methanol/chloroform (Fisher) and run on Silica Gel GHLF plates (Uniplate, Analtech, Newark, Del.) and visualized under ultraviolet light in an EpiChemi³ Darlkoom (UVP, Upland, Calif.) and also under iodine vapor (Fisher).

Mixed Lymphocyte Culture.

B6 black and balb/c mice (Jackson Laboratories, Bar Harbor, Me.) were sacrificed by cervical dislocation and mesenteric lymph nodes and spleens were collected. Cells were teased out from the collected tissues in RPMI (BioSource) and washed twice with the same. A single cell suspension containing 2×106 cells/ml for each strain was prepared in RPMI containing 10% supplemented calf serum (Hyclone). The two cell suspensions were mixed and 100 μl of the mix was dispensed into sterile 96 well plates (Sarstedt). 100 mM solutions of compounds were prepared in DMSO. For each compound, 20 μl of 5% bovine serum albumin (Sigma) was used to aid the dissolution process when adding the DMSO solution to RPMI. A DMSO-BSA-solution solution of candidate compounds (100 μl) was added to the wells such that the final concentration was 100 μM while the volume percentage of the vehicle in which the compound was dissolved, DMSO (Sigma), was 0.1%. Analogously, wells containing 10, 1, and 0.1 μM candidate compound were also prepared with DMSO concentration 0.01, 0.001, and 0.0001% v/v. Blank DMSO controls were run for each compound/DMSO concentration (positive control), and mitomycin (Sigma, 25 μg/well) controls were run for each compound/DMSO concentration (negative control). The 96 well plates were incubated at 37° C., 5% CO₂ in an IncuSafe Incubator (Sanyo, Osaka, Japan). Compounds, blank DMSO, and mitomycin were replaced at 24 h. 1 μCu [³H] thymidine (Perkin Elmer, Boston, Mass.) diluted in 30 μl RPMI was added to each well at 42 h, and 6 h later, cells were harvested on a cell harvester (Inotech, Rockville, Md.) onto Glass Fibre Filter Type G-7 (Inotech). Filters were treated with methanol (Fisher) washed 3 times with PBS, and finally with methanol again. Filter papers were air-dried and the radioactivity was counted in Econosafe Scintillation Cocktail (Research Products International, Mount Prospect, Ill.) on a LS6500 counter (Beckman, Fullerton, Calif.). Percent inhibition was calculated as: [1−(sample cpm−mitomycin cpm)/(DMSO cpm−mitomycin cpm)]×100

Cell Viability. For each condition in the mixed lymphocyte assay, an additional well was prepared and was exposed to exactly the same conditions as above. The cells in these wells were resuspended at 48 h and scored for viability by the Trypan Blue Exclusion Assay. Briefly, an equal volume of 0.2% w/v trypan blue (Sigma) solution in PBS was added to the resuspended cells, and these were counted using a Hemacytometer (Bright-Line, Horsham, Pa.) on a CK2 microscope (Olympus, Tokyo, Japan).

Results and Discussion

Inhibition of the Phosphorylated ITAM2 Peptide Association with Lck SH2

Domain.

The compounds were initially tested for their ability to inhibit p56 Lck SH2 domain association with phosphotyrosine-containing C-terminal ITAM2 peptide. Immunoblots from the inhibition assays for compounds 92, 232, 239, 245, 262, 264, 276 and 73 are shown in FIG. 1. The inhibition of p56 Lck is reflected by the decreased intensity of the blots as compared to the control. FIG. 1 (panel A) shows that compounds 92, 232, 239, 245, 262, 264 and 276 have significant inhibitory activities at 100 μM. FIG. 1 (panel B) shows a dose dependent inhibition of co-precipitation by the inhibitor 73; at 40 μM (lane 5) the compound significantly blocked p56 Lck association with the ITAM2 peptide. The 34 preferred compounds identified herein were shown to have significant inhibitory activity at 100 μM. These compounds are the following.

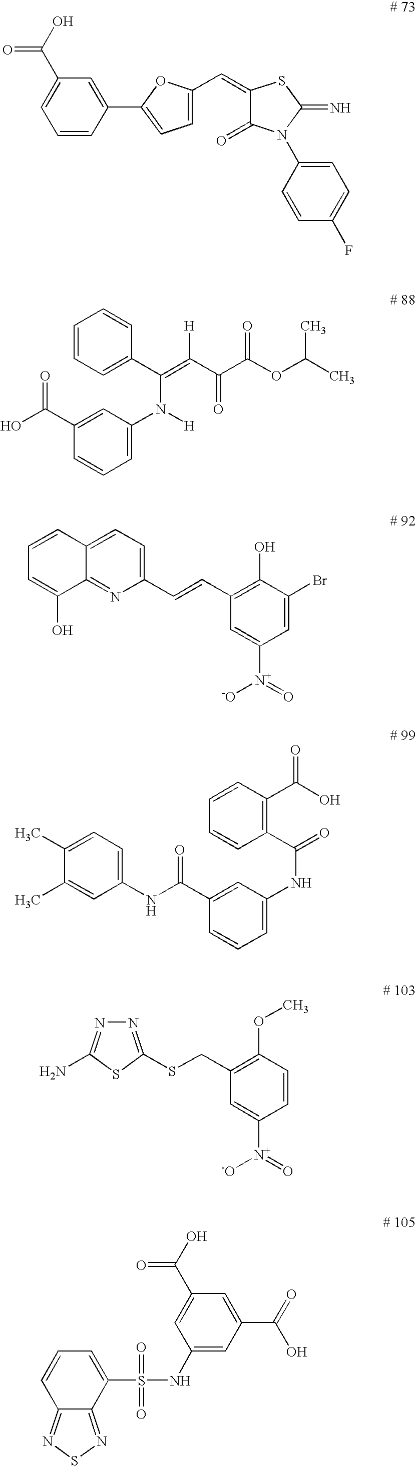

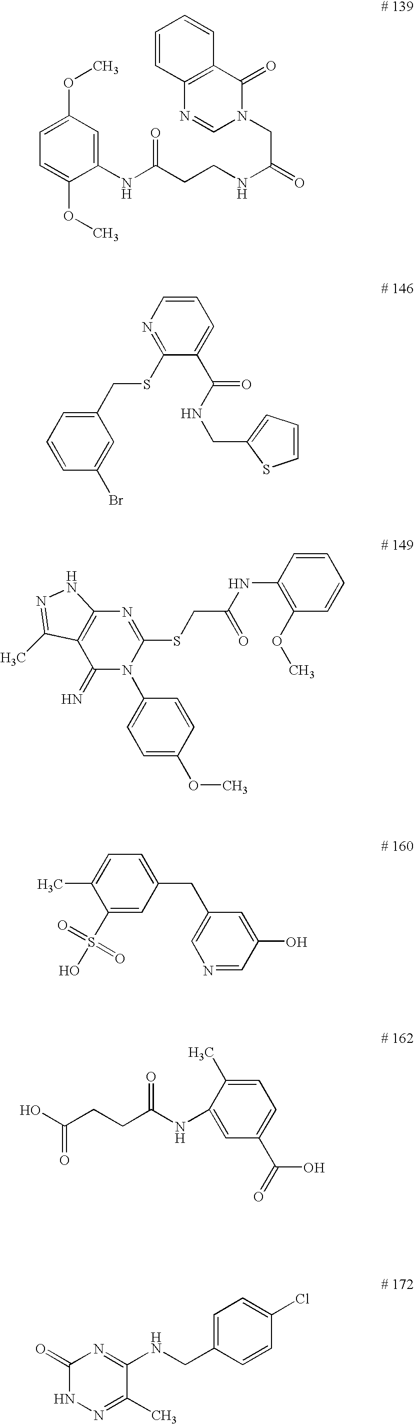

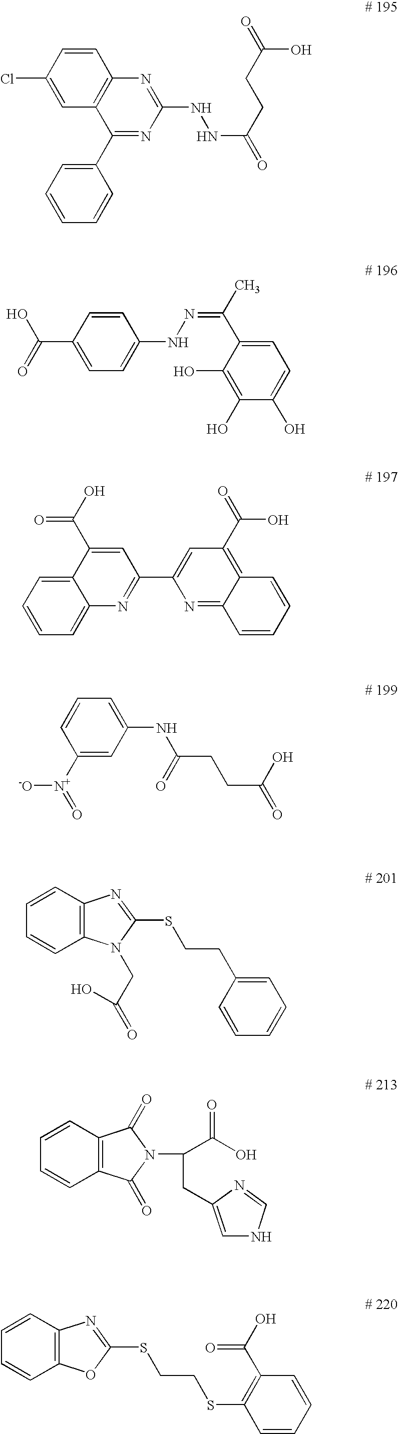

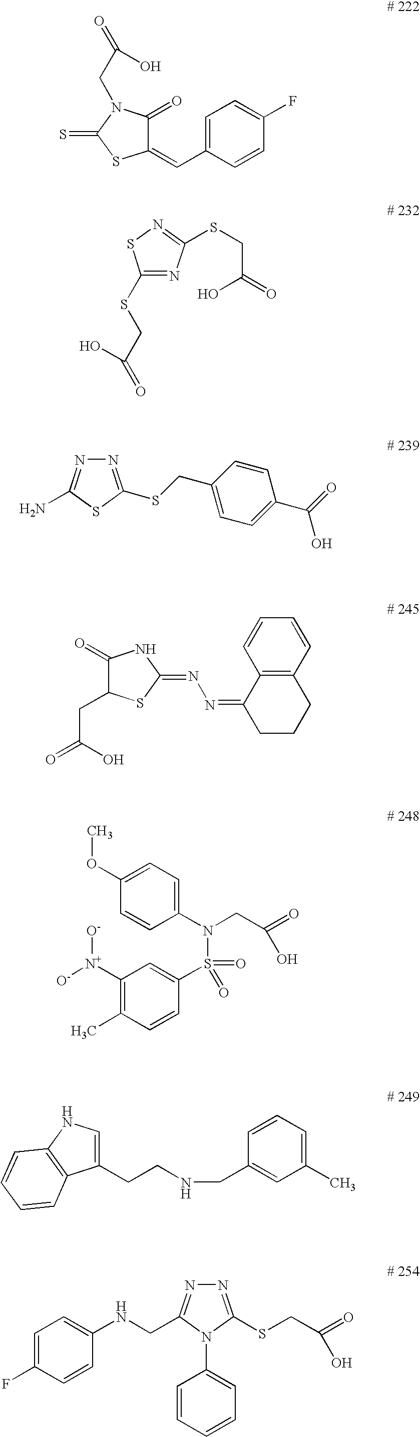

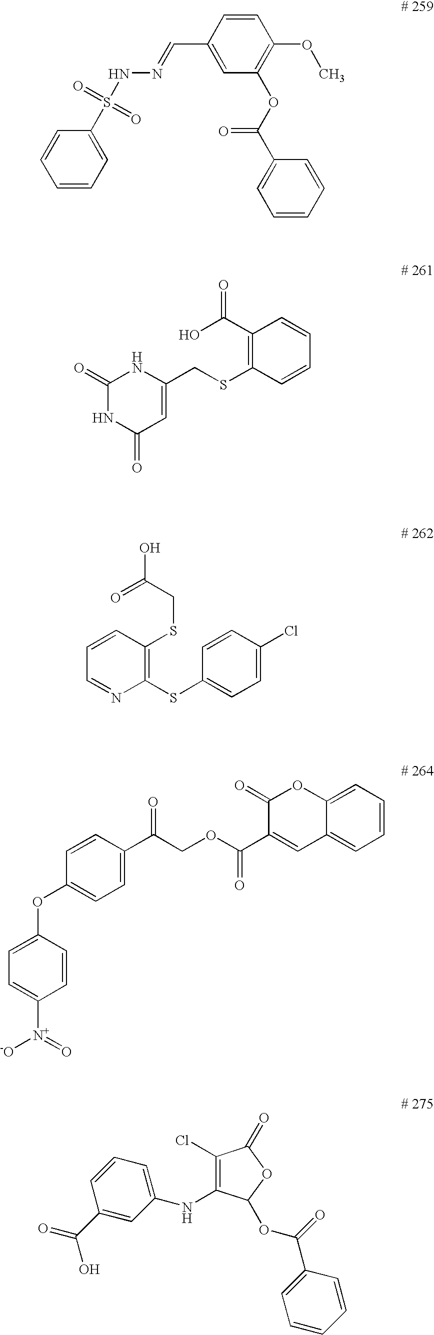

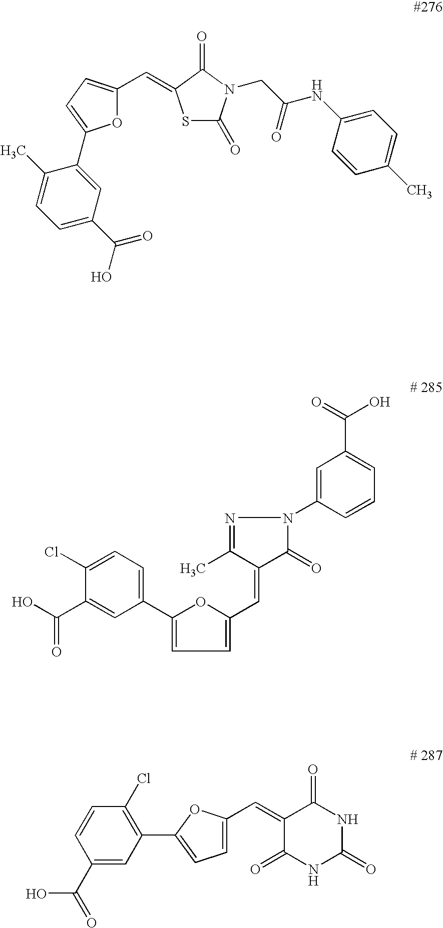

Of these, compounds 73 and 92 show strong inhibitory activity at 40 and 10 μM, respectively.

Inhibitory Activity in Mixed Lymphocyte Culture Assay.

Mixed lymphocyte culture assays were performed where lymphocytes from two different strains of mice with different histocompatibility antigens were mixed. Due to the difference in the histocompatibility antigens, resting T cells from both strains of mice will undergo blast transformation and propagate. As in any T cell activation process, the activation of Lck is essential. Therefore, the modulation of activation of Lck can be quantified as the downstream modulation in the levels of ³H-TdR incorporation into DNA. 24 out of the 34 identified compounds were tested in vivo, with 13 compounds showing inhibitory activity at a 100 μM concentration (FIG. 2). Thus, over 50% of the compounds identified by the in vitro assay also show activity in the cellular functional assay at the conditions described herein. These compounds represent the most promising leads for further development. For 7 compounds, biphasic activity was observed in the mixed lymphocyte culture assay, where positive inhibitory activity is observed at higher concentration (100 μM) and negative inhibition (i.e. activation) occurs at lower concentrations (1 μM). Such effects may be associated with the regulatory mechanism of p56 Lck.

The SH2 domain is involved in both the recruitment of substrates and auto-regulation of p56 Lck. It has been postulated that the Lck SH2 domain can bind with Lck C tail domain containing the phosphorylated Tyr505 to create an inactive “closed” conformation. The intramolecular interaction between C tail and SH2 domain responsible for inactivation of Lck kinase is quite weak as compared with the binding of the SH2 domain to optimal high-affinity ITAMs.⁴³⁻⁴⁵ High affinity inhibitors (e.g. 73 and 92), may block the intermolecular interaction at all tested concentrations, leading to inhibition in the mixed lymphocyte culture assay. Biphasic compounds (e.g. 99 and 139) at lower concentrations may only block the intramolecular interaction that leads to activation in the mixed lymphocyte culture assay, while at higher concentrations, they may block the intermolecular interaction, leading to inhibition in the mixed lymphocyte culture assay.

### Assay Example 1

Jurkat Cell Activation-Dependent Phosphorylation

Phosphorylation in Jurkat cells activated by the monoclonal antibody, OKT-3, is correlated with T-cell activation.

A. Compounds are tested for their effect on OKT-3 activated Jurkat cells. OKT-3 is a monoclonal antibody against CD3-ε chain. Treatment of Jurkat cells with OKT-3 antibody for 5 min at 37° C. activates Jurkat cells and induces tyrosine phosphorylation of several cellular proteins. In JCaM1.6 Jurkat cells expressing p56^{lck} with defective kinase activity, the OKT-3-mediated phosphorylation of cellular proteins does not take place, indicating that p56^{lck} plays an essential role in this process. Experiments are carried out to determine if compounds of the invention, or known compounds which can serve as standards, affect cellular tyrosine phosphorylation stimulated by OKT-3 in Jurkat cells.

The assay is performed as follows: Jurkat cells (1×10⁶ cells) in RPMI 1640 supplemented with 10% fetal bovine serum (FBS) are treated with OKT-3 antibody (0.2 μg) and compounds (100 μM) for 5 min at 37° C. Cultures are immediately placed on ice after the incubation period, washed 3 times with cold PBS and lysed in 20 μl of SDS-sample buffer. Samples are briefly sonicated and boiled for 5 min and applied to SDS-PAGE (12% gel). After SDS-PAGE, proteins are blotted onto Immobilon P membrane (Millipore) for western blot analysis. Membranes are blocked overnight with 5% dried milk in TBST (Tris buffered saline containing 1% triton X-100). After washing, blots are incubated with monoclonal anti-phosphotyrosine antibody for 1 h in TBST followed by 1 hr incubation with horseradish peroxidase (HRP)-conjugated goat anti-mouse IgG. Blots are developed using enhanced chemiluminescence (ECL, Pierce).

B. Dose response experiments are performed.

The assay is performed as follows: Western blot analyses using anti-phosphotyrosine antibody are performed. Compounds are added at concentrations of 0.1, 1, 10 and 100 μM. Cells are cultured as above.

### Assay Example 2

Effect on Jurkat Cell Viability

Compounds can be tested to determine whether they are cytotoxic or cytostatic. To test whether a compound which inhibits OKT-3-mediated stimulation of tyrosine phosphorylation has a cytotoxic effect on Jurkat cells, its effect on the growth of Jurkat cells is tested.

### AssaY Example 3

Inhibition of IL-2 Production in Jurkat Cells

Interleukin 2 (IL-2) is an autocrine growth factor for T cells, the production of which requires T cell antigen receptor and co-receptor generated activation signals in normal T cells. The production of IL-2 is a hallmark of T cell activation signaling, leading to the clonal expansion of antigen-specific T cell clones. In Jurkat cells, treatment of cells with OKT-3 leads to the production of IL-2. In order to test whether a compound which inhibits OKT-3 induced tyrosine phosphorylation of cellular proteins also inhibits IL-2 production, Jurkat cells are treated with OKT-3 in the presence or absence of the compound.

The assay is performed as follows: Jurkat cells (1×10⁶ cells) are treated with OKT-3 antibody (0.2 μg) in the presence or absence of compounds (0.1, 1 and 10, μM) for 24 h in RPMI 1640+10% FBS. At the end of the incubation period, culture media are harvested and assayed for human IL-2 by RIA. Cells treated with OKT-3 only and PMA+lonomycin serves as a positive control. Untreated cells serve as a negative control. IL-2 production is not detected in untreated cells.

### Assay Example 4

Binding to the SH2 Domain Using [³H]-Compound

Purified recombinant p56^{lck}-SH2 domain expressed as a GST-fusion protein in bacteria is bound to either anti-GST agarose beads or glutathione-agarose beads. These beads bind the GST-p56^{lck}-SH2 protein and facilitate the separation of [³H]-test compound bound SH2 domain from unbound compound. Alternatively,. dextran-coated activated charcoal solution is used to separate bound from unbound compounds.
Determination of K_{D}: The binding affinity of a compound is determined by applying standard Scatchard analysis where the binding assay is performed in the presence of a fixed amount of the [³H]-compound and an increasing amount of cold compound. The K_{D} of the compound is calculated using the Ligand Program (Munson & Rodbard 1980), Analytical Biochem., 107,220-239. The co-P is evident from, e.g., Sun et al., 1987, Biochem. Biophys. Res. Comm. 148, 603-608.
Determination of IC₅₀: In addition to the determination of K_{D}, the IC₅₀ value for the compounds to inhibit p56^{lck}-SH2 domain binding to the N-terminal pY of CD3 ζITAM2 is measured. The SH2 domain of p56^{lck} has the highest binding affinity (0.1 μM) to the N-terminal pY of the second ITAM of CD3 ζ chains. A synthetic peptide corresponding to this region is made and conjugated to agarose beads (ζ-NpY-ITAM2-agarose). P56^{lck} binds to ζ-NpY-ITAM2-agarose through its SH2 domain and can be precipitated. Using the recombinant p56^{lck} GST-SH2 protein, binding assays using ζ-NpY-ITAM2-agarose are carried out in the presence or absence of the compounds at various concentrations (0.01˜100 μM) in RIPA buffer. After 2 h of the binding reaction at room temperature, beads are harvested and washed three times with RIPA buffer. The relative amounts of GST-SH2 bound to the ζ-NpY-ITAM2-agarose are measured by a colorimetric assay after incubating the beads with anti-GST antibody and HRP-conjugated secondary antibody and HRP enzyme substrate. The OD read out from the binding of GST-SH2 to the ζ-NpY-ITAM2-agarose in the absence of the compound serves as positive control and represents the 100% bound level. Background (ζ-NpY-ITAM2-agarose without GST-SH2) is subtracted from each value. Assays are carried out in triplicate and the average from three independent assays are used to calculate IC₅₀ values for each compound.

### Assay Example 5

Co-Immunoprecipitation Experiments

The binding of compounds to the p56^{lck} SH domain pY+3 pocket affects the SH2-mediated interaction of p56^{lck} with phosphotyrosine residues of cellular target proteins. For example, the treatment of cells with an inhibitor compound inhibits the association of p56^{lck} with CD3ζand ZAP-70, whereas an increase in the association occurs following treatment with a stimulator compound. The presence of these molecular interactions is assessed by co-immunoprecipitation assays in activated Jurkat cells in the presence or absence of the compounds. Using this assay (in conjunction with p56^{lck} kinase assays as described elsewhere herein), one can estimate the IC₅₀ of a compound for blocking the molecular interactions and the ED₅₀ of a compound for activating kinase activity. These values are generally in the vicinity of the K_{D} of the compound binding to the SH2 domain, provided that the effect of the compound is mediated by binding to the SH2 domain of p56^{lck}.

Jurkat cells (5×10⁶ to 1×10⁷/ml; 1 ml/condition) are activated using OKT-3 antibody (1 μg/5×10⁶ cells) in the presence or absence of the compound to be tested (0.01 to 100 μm). Following 10 min of activation at 37° C., cells are harvested and lysed using RIPA or NP-40 lysis buffer. After removing insoluble materials, the supernatant is treated with 1 to 2 μg of antibody against either CD3ζor ZAP-70. Immune complex is precipitated using Protein A or G conjugated agarose beads (10 μl of 50% slur) at 4° C. Beads are harvested and washed three times with lysis buffer, boiled for 2 min., resolved on SDS PAGE (e.g., 12.5%) and blotted on Immobilon-P membrane for Western blot analysis, using anti-p56^{lck} antibody. Samples from non-activated cells and activated cells in the presence of the compound serve as negative and positive controls. Blots are also re-probed with the precipitating antibody to ensure that equal amounts of protein (either CD3ζ or ZAP-70) precipitates from each sample. The presence of co-immunoprecipitation p56^{lck} is examined. A semi-quantitative analysis can also be carried out by altering the compound concentrations in the assay and measuring the relative amount of co-immunoprecipitation p56^{lck}. Quantitation is performed by performing EIA (using spectrophotometric assay following HRP-conjugated 2° antibody+chromogenic substrate), chemoluminescence (using image analyzer following HRP-conjugated 2° antibody+ECL) or phosphor image analysis (using [¹²⁵I]-2° antibody). IC₅₀ values for compounds are determined.

### Assay Example 6

Demonstration of Specificity of Binding

Based on sequence homology and other considerations five additional SH2 domain containing kinases, including ZAP-70, which contains two pY binding sites, are selected for testing of specificity, both computationally and experimentally. These proteins are listed in Table 1:

TABLE 1
SH2 domains used for determination of selectivity

| SH2 domain | EF loop | FB loop | βG strand | PDB Entry |
|---|---|---|---|---|
| lck | Ser-Pro-Arg | Pro | Arg-Pro-Cys | 1LKK |
| Hck | Ser-Pro-Arg | Ser | Val-Pro-Cys | 1QCF |
| Fyn | Thr-Thr-Arg | Glu | Val-Pro-Cys | 1AOT |
| Src | Thr-Ser-Arg | Ser | Asn-Val-Cys | 1BKL |
| Shc | Lys-Asp | Glu | Gln-Pro-Val | 1TCE |
| ZAP-70, N^{a} | Ala-Gly-Gly | Cys | Lys-Pro-Cys | NA^{b} |
| ZAP-70, C^{a} | Pro-Glu-Gly | Asp | Glu-Ala-Cys | NA^{b} |

^{a}ZAP-70 contains 2 phosphotyrosine binding sites in the N and C-terminal regions, designated ZAP-70, N and ZAP-70, C, respectively
^{b}NA: Not available from the PDB, however, the structure was obtained by the authors.

Specificity is determined via differential binding of the SH2 domains in Table 1 with the phosphopeptides listed in Table 2. The SH2 domains are selected based on homology with p56^{lck} as well as the availability of 3D structural data. Phosphopeptides are selected based on their SH2 domain specificity. The ζ-ITAM-2-C and ζ-ITAM-1 peptides are specific for p56^{lck} and ZAP-70, respectively. All Src kinases including p56^{lck} and Shc are known to bind with similar affinity to the hamster polyoma middle T antigen peptide that contains the pYEEI sequence.

TABLE 2

Phosphopeptides used in the selectivity
binding experiments

| | |
|---|---|
| pY Peptide | SH2 domain |

| | | |
|---|---|---|
| SEQ ID No. 1 | TATEGQpYQPQP (ζ-ITAM-2-C) | p56^{lck} |
| SEQ ID No. 2 | GQNQLpYNELNLGRREEpYDVLDKR | ZAP-70 |
| | (ζ-ITAM-1) | |
| SEQ ID No. 3 | EQpYEEIPIA (hamster | p56^{lck,} |
| | polyoma middle T) | Src, Hck, |
| | | Shc, Fyn |

A solid phase binding competition assay between the compounds and the phosphopeptides is used. SH2 domains are PCR amplified and expressed as GST-fusion proteins in E. coli. The GST-fusion protein is purified on a glutathione column. SH2 domains are cleaved off using thrombin and purified on a gel filtration column. Synthetic phosphotyrosine containing peptides listed in Table 2 are synthesized using conventional methods. These peptides are biotinylated at the N-terminus, away from the SH2 docking site, using kits available from Pierce (Rockford, Ill.). A 96-well EIA plate is coated with a purified SH2 domain (1 ,μg/well; ˜100 nmol). Biotinylated peptide (˜200 nmol) in the presence or absence of varying concentrations (pmol to μmol) of compound is added. Biotinylated peptide bound to the SH2 domain is then measured in various ways, such as by colorimetric assay using HRP-streptavidin+substrate, by fluorescence using FITC-labeled streptavidin or by scintillation counter using [¹²⁵I]-labeled streptavidin. OD (or cpm) reading of ζ-ITAM2-C peptide without compound constitutes 100% bound. OD (or cpm) reading of SH42 only constitutes the blank. IC₅₀ values for each compound are determined. Alternatively, the assay is adapted to use glutathione-agarose beads to separate SH2 domain after the incubation period. K_{D} for each compound is determined using binding competition assay.

### Assay Example 7

Mixed Lymphocyte-Culture Assay

Another readout assay that is used to measure biological response is a mixed lymphocyte culture assay in which lymphocytes from two. different strains of mice with different histocompatibility antigens are mixed. Due to the difference in the histocompatibility antigens, resting T cells from both strains of mice undergo blast transformation and propagate. As in any T cell activation process, the activation of p56^{lck} is essential. Therefore, the modulation of p56^{lck} activity can be quantified as the downstream modulation in the levels of [³H]-TdR incorporation into DNA.

Lymph node or splenic lymphocytes are harvested from two different allogeneic strains of mice. Cells (1×10⁶) from each strain are mixed in 96 well culture plates containing 200 μl of culture medium in the presence or absence of the compound (0.1˜100 μM) and cultured for 72 h. Six hours before harvest, 0.5 μCi of [³H]-TdR is added to each well. At the end of the culture period, cells are harvested on a glass fiber filter using a cell harvester. Filters are washed with PBS and then with ethanol and [³H]-TdR incorporated into DNA is measured using scintillation counting. Experiments are carried out in triplicate. Cells cultured in the absence of the compound serve as a positive control. Cells from each strain of mouse cultured in the absence of allogeneic lymphocytes serve as the negative control. The compounds are added every 12 h. The compounds that inhibit protein phosphorylation and IL-2 production have a similar effect on [³H]-TdR incorporation.

### Assay Example 8

Inhibition in vivo of Immune Response of Delayed-Type Hypersensitivity (DTH) and Anti-Type II Collagen-Induced Rheumatoid Arthritis in Mice

One experimental model is DTH response to PPD. DTH reaction is a typical T cell immune response and, thus, well suited to be used for assessing the in vivo effect of the compounds. Mice are immunized using BCG in complete Freund's adjuvant (CFA). After the initial immunization, a tuberculin skin test is performed. Bilateral regions of BCG immunized mouse skin are treated with hair removal cream. Interdermal injection of tuberculin is administered in two hair-removed sites. The compound dissolved in DMSO is applied to one of the sites every 12 h. The other site is treated with DMSO only and serves as an internal positive control. After one week, diameters of DTH reactions for both control and treated sites are measured. Five mice are used in a group. Statistical analysis is carried out to determine effective dose required for 50% reduction in the.DTH skin test over control. Treatment is carried out with animals anesthetized (using vapolizer, 2% isoflurane). Animals are kept under anesthesia till DMSO is completely absorbed to the skin. The amount of DMSO applied to each site is kept minimal (˜10 μl).

In addition to the DTH reaction, experiments are performed using collagen-induced arthritis (CIA) in mice. CIA is an experimental model for rheumatoid arthritis (RA) in human. A commercial kit is available to reliably induce CIA in mice in a short period of time. Monoclonal antibody cocktail against type II collagen is injected into mice, i.v. RA develops within 24-48 h after injection of the antibody cocktail and exacerbated swelling of the two hind paws becomes evident by day 6. One of the paws from each animal is treated with the compound dissolved in DMSO. The other paw is treated with DMSO only and serves as an internal positive control. Swelling of the paws is measured. Thickness of the paws from saline injected, DMSO-treated control mice serves as negative control. Hind paws are dipped in DMSO solution (with or without the compound to be tested) for 10 seconds twice daily at 12 h intervals. Animals are kept under anesthesia till DMSO is completely absorbed. Five mice are used in a group. Statistical analysis is carried out to determine the effective dose required for 50% reduction in the swelling of paws over the control for the inhibitory compounds. Treatment is carried out using mice under anesthesia.

From the foregoing description, one skilled in the art can easily ascertain the essential characteristics of this invention, and without departing from the spirit and scope thereof, can make changes and modifications of the invention to adapt it to various usage and conditions.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosure of all applications, patents and publications, cited herein are hereby incorporated in their entirety by reference.

### BRIEF DESCRIPTION OF DRAWINGS

FIG. 1. Immunoblots from compounds inhibiting p56 Lck SH2 domain association with phosphotyrosine-containing ITAM2 peptide. The inhibition of p56 Lck is reflected by the decreased intensity of the blots as compared to the control. A: Lane 1: control; Lane 2-8: compounds 92, 232,.239, 245, 262, 264 and 276 at 100 μM, respectively. B: Lane 1: control; Lane 2-8: compound 73 at 10, 20, 30; 40, 60, 80 and 100 μM, respectively. FIG. 2. Percent inhibition of ³H-thymidine uptake in mixed lymphocyte culture by 13 compounds selected by in vitro screening. Legend: Black bars: 100 μM; grey bars: 10 μM; white bars: 1 μM. Compounds 73 and 92 were not tested at 100 μM concentration due to solubility issues. Each bar represents mean±standard deviation of three replicates.

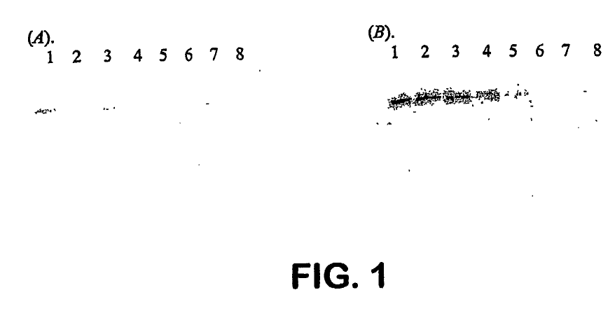

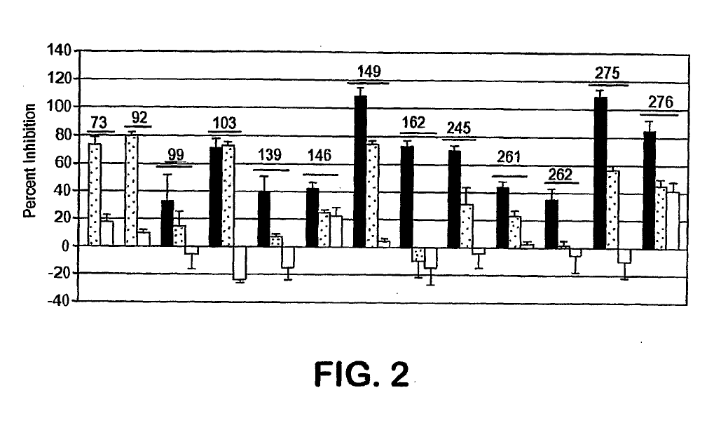

### REFERENCES

(1) Tong, L.; Warren, T. C.; King, J.; Betageri, R.; Rose, J.; Jakes, S. Crystal structures of the human p56^{lck} SH2 domain in complex with two short phosphotyrosyl peptides at 1.0 A and 1.8 A resolution. J. Mol. Biol. 1996, 256, 601-610.
(2) Broadbridge, R. J.; Sharma, R. P. The Src Homology-2 Domains (SH2 domains) of the protein tyrosine kinase p56lck: structure, mechanism and drug design. Current Drug Targets 2000, 1, 365-386.
(3) Straus, D. B.; Weiss, A. Genetic evidence for the involvement of the lck tyrosine kinase in signal transduction through the T cell antigen receptor. Cell 1992, 70, 585-593.
(4) Weiss, A.; Littman, D. R. Signal transduction by lymphocyte antigen receptors. Cell. 1994, 76, 263-274.
(5) Straus, D. B.; Chan, A. C.; Patai, B.; Weiss, A., SH2 domain function is essential for the role of the Lck tyrosine kinase in T cell receptor signal transduction. J Biol. Chem. 1996, 271, 9976-9981.
(6) Pawson, T.; Gish, G. D. SH2 and SH3 domains: from structure to function. Cell. 1992, 71, 359-362.
(7) Cody, W. L.; Lin, Z.-W.; Panek, R. L.; Rose, D. W.; Rubin, J. R. Progress in the development of inhibitors of SH2 domains. Current Pharmaceutical Design 2000, 6, 59-98.
(8) Cousins-Wasti, R.; Ingraham, R. H.; Morelock, M. M.; Grygon, C. A. Determination of affinities for lck SH2 binding peptides using a sensitive fluorescence assay: comparison between the pYEEIP and pYQPQP consensus sequences reveals context-dependent binding specificity. Biochemistry. 1996, 35, 16746-16752.
(9) Sawyer, T. K. Src homology-2 domains: structure, mechanisms, and drug discovery. Biopolymers. 1998, 47, 243-261.
(10) Beaulieu, P. I.; Cameron, D. R.; Ferland, J. M.; Gauthier, J.; Ghiro, E.; Gillard, J.; Gorys, V.; Poirier, M.; Rancourt, J.; Wemic, D.; Llinas-Bnimet, M.; Betageri, R.; Cardozo, M.; Hickey, E. R.; Ingraham, R.; Jakes, S.; Kabcenell, A.; Kirrane, T.; Lukas, S.; Patel, U.; Proudfoot, J.; Sharma, R.; Tong, L.; Moss, N. Ligands for the tyrosine kinase p56lck SH2 domain: discovery of potent dipeptide derivatives with monocharged, nonhydrolyzable,, phosphate replacements. J. Med. Chem. 1999, 42, 1757-1766.
(11) Lee, T., R.; Lawrence, D. S. SH2-directed ligands of the Lck tyrosine kinase. J. Med. Chem. 2000, 43, 1173-1179.
(12) Kuriyan, J.; Cowburn, D. Modular peptide recognition domains in eukaryotic signaling. Annu. Rev. Biophys. Biomol. Struct. 1997, 26, 259-288.
(13) Songyang, Z.; Cantley, L. C. Recognition and specificity in protein tyrosine kinase-mediated signalling. Trends Biochem. Sci. 1995, 20, 470-475.
(14) Marengere, L.; Songyang, Z.; Gish, G. D.; Schaller, M. D.; Parsons, J. T.; Stem, M. J.;

Cantley, L. C.; Pawson, T. SH2 domain specificity and activity modified by a single residue. Nature. 1994, 369, 502-505.
(15) Songyang, Z.; Gish, G.; Mbamalu, G.; Pawson, T.; Cantley, L. C. A single point mutation switches the specificity of group III Src homology (SH2) domains to that of group I SH2 domains. J. Biol., Chem. 1995, 270, 26029-26032.
(16) Brennan, M. B. Drug Discovery Filtering Out Failures Early in the Game. Chemical & Engineering News 2000, 78, 63-73.
(17) Estrada, E.; Uriarte, E.; Montero, A.; Teijeira, M.; Santana, L. D.; Clercq, E. A novel approach for the virtual screening and rational design of anticancer compounds. J Med. Chem. 2000, 43, 1975-1985.
(18) Baxter, C. A.; Murray, C. W.; Waszkowycz, B.; Li, J.; Sykes, R. A.; Bone, R. G.; Perkins, T. D.; Wylie, W. New approach to molecular docking and its application to virtual screening of chemical databases. J. Chem. Inf. Comput. Sci. 2000, 40, 254-262.
(19) Walters, W. P.; Stahl, M. T.; Murcko, M. A. Virtual screening—an overview. Drug Discov. Today 1998, 3, 160-178.
(20) Massova, I.; Martin, P.; Bulychev, A.; Kocz, R.; Doyle, M.; Edwards, B. F.; Mobashery, S. Templates for design of inhibitors for serine proteases: application of the program DOCK to the discovery of novel inhibitors for thrombin. Bioorg. Med. Chem. Lett. 1998, 8, 2463-2466.
(21) Li, S.; Gao, J.; Satoh, T.; Friedman, T. M.; Edling, A. E.; Koch, U.; Choksi, S.; Han, X.; Komgold, R.; Huang, Z.-A computer screening approach to immunoglobulin superfamily structures and interactions: discovery of small non-peptidic CD4 inhibitors as novel immunotherapeutics. Proc. Natl. Acad. Sci. U S A. 1997, 94, 73-78.
(22) Chen, I.-J.; Neamati, N.; Nicklaus, M. C.; Orr, A.; Anderson, L.; Barchi, J. J. J.; Kelley, J. A.; Pommier, Y.; MacKerell, A. D. J. Identification of HIV-1 integrase inhibitors via three-dimensional database searching using ASV and HIV-1 integrases as targets. Bioorg. Med. Chem. 2000, 8, 2385-2398.
(23) Deborah, A. L.; Willian, V. M.; Linda, K. J. Application of Virtual screening tools to a protein-protein interaction: database mining studies on the growth hormone. Med. Chem. Res. 1999, 9, 579-591.
(24) Pan, Y.; Huang, N.; Cho, S.; MacKerell, A. D., Jr. Consideration of Molecular Weight during Compound Selection in Virtual Target-Based Database Screening. J Chem. Inf. Comput. Sci. 2003, 43, 267-272.
(25) SYBYL; 6.7 ed.; Tripos Associates: St. Louis. Mo.
(26) Gasteiger, J.; Rudolph, C.; Sadowski, J. Automatic generation of 3D-atomic coordinates for organic molecules. Tetrahedron Comput. Methodol. 1990, 3, 537.
(27) Berman, H. M.; Westbrook, J.; Feng, Z.; Gilliland, G.; Bhat, T. N.; Weissig, H.; Shindyalov, I. N.; Bourne, P. E. The Protein Data Bank. Nucleic Acids Research 2000, 28, 235-242.
(28) Meng, E. C.; Shoichet, B. K.; Kuntz, I. D. Automated docking with grid-based energy evaluation. J. Comput. Chem. 1992, 13, 505-524.
(29) Leach, A. R.; Kuntz, I. D. Conformational analysis of flexible ligands in macromolecular receptor sites. J. Comput. Chem. 1992, 13, 730-748.
(30) Connolly, M. L. Solvent-accessible surfaces of proteins and nucleic acids. Science 1983, 221, 709-713.
(31) Ferrin, T. E.; Huang, C. C.; Jarvis, L. E.; Langridge, R. The MIDAS display system. J. Mol. Graphics 1988, 6, 13-27.
(32) Goodford, P. J. A computational procedure for determining energetically favorable binding sites on biologically important macromolecules. J. Med. Chem. 1984, 28, 849-857.
(33) Ewing, T. J. A.; Kuntz, I. D. Critical evaluation of search algorithms used in automated molecular docking. J Comput. Chem. 1997, 18, 1175-1189.
(34) Oprea, T. I. Property distribution of drug-related chemical databases. J. Comput. Aided Mol. Des. 2000, 14, 251-264.
(35) Oprea, T. I.; Davis, A. M.; Teague, S. J.; Leeson, P. D. Is There a Difference between Leads and Drugs? A Historical Perspective. J. Chem. Inf Comput. Sci. 2001, 41, 1308-1315.
(36) Teague,. S. J.; Davis, A. M.; Leeson, P. D.; Oprea, T. I. The design of leadlike combinatorial libraries. Angew. Chem., Int. Ed. 1999, 38, 3743-3748.
(37) Godden, J. W.; Xue, L.; Bajorath, J. Combinatorial preferences affect molecular similarity/diversity calculations using binary fingerprints and Tanimoto coefficients. J. Chem. Inf. Comput. Sci. 2000, 40, 163-166.
(38) MOE; 2002 ed.; Chemical Computing Group Inc.: Montreal, Quebec, Canada.
(39) Lipinski, C. A.; Lombardo, F.; Dominy, B. W.; Feeney, P. J. Experimental and computational approaches to estimate solubility and permeability in drug discovery development settings. Adv. Drug Delivery Research 1997, 23.
(40) Su, A-L.; Lorber, D. M.; Weston, G. S.; Baase, W. A.; Matthew, B. W.; Schoichet, B. K. Docking molecules by families to increase the diversity of hits in database screening: computational strategy and experimental evaluation. Proteins 2001, 42, 279-293.
(41) Kubinyi, H. Structure-based design of enzyme inhibitors and receptor ligands. Current Opinion in Drug Discovery and Development 1998, 1, 4-15.
(42), Shoichet, B. K.; Leach, A. R.; Kuntz, I. D. Ligand solvation in molecular docking. PROTEINS: Structure, Function and Genetics. 1999, 34, 4-16.
(43) Payne, G.; Stolz, L. A.; Pei, D.; Band, H.; Shoelson, S. E.; Walsh, C. T. The phosphopeptide- binding specificity of Src family SH2 domains. Chemistry and Biology 1994, 1, 99-105.
(44) Songyang, Z.; Shoelson, S. E.; Chaudhuri, M.; Gish, G.; Pawson, T.; Haser, W. G.; King, F.; Roberts, T.; Ratnofsky, S.; Lechleider, R. J.; Neel, B. G.; Birge, R. B.; Fajardo, J. E. F.; Chou, M. M.; Harafusa, H.; Schaffhausen, B.; Cantley, L. C. SH2 domains recognize specific phosphopeptide sequences. Cell 1993, 72, 767-778.
(45) Liu, X.; Brodeur, S. R.; Gish, G.; Songyang, Z.; Cantley, L. C.; Laudano, A. P.; Pawson, T. Regulation of c-Src tyrosine kinase activity by the Src SH2 domain. Oncogene 1993, 8, 1119-1126.
(46) Humphrey, W.; Dalke, A.; Schulten, K. VMD--visual molecular dynamics. J Mol. Graphics 1996, 14, 33-38.

We claim:

1. A method of achieving an immunomodulatory effect, achieving an antineoplastic effect, or inhibiting hyperproliferative cell growth in a patient in need thereof, comprising administering to said patient an effective amount of a compound formulae I to XVII or a pharmaceutically acceptable salt thereof 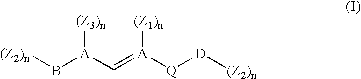
wherein,
B is a phenyl ring,
D is a phenyl ring or a 5-membered saturated or partially or fully unsaturated heterocyclic ring containing 1, 2 or 3 heteroatoms selected from O, S, and N,
A is, in each case independently of each other, a 5-membered saturated or partially or fully unsaturated heterocyclic ring containing 1, 2 or 3 heteroatoms selected from O, S, and N,
Q is a bond or an alkylene or alkenylene group containing 1-5 carbon atoms, which is optionally substituted with ═O, and in which optionally a carbon atom is replaced with an N atom,
Z₁ is, in each case independently, —NH₂, ═O, ═NH, or ═N-phenyl, -phenyl, or alkyl containing 1 to 5 carbon atoms,
Z₂ is, in each case independently, —OH, halogen, alkyl containing 1-5 carbon atoms, which is optionally substituted with halogen, and/or substituted with ═O and/or —OH, and in which one C atom is optionally replaced with and O atom,
Z₃ is, in each case independently, alkyl containing 1-5 carbon atoms, and
n is, in each case independently, 0, 1, 2, or 3; 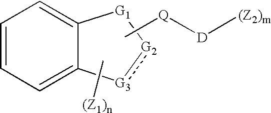
wherein,
G₁, G₂, and G₃ are, in each case independently, C, O, S, or N,
D is a phenyl ring or a 5- or 6-membered saturated or partially or fully unsaturated heterocyclic ring containing 1, 2 or 3 heteroatoms selected from O, S, and N,
Q is a straight chain or branched alkylene or alkenylene group containing 1-5 carbon atoms, which is optionally substituted with ═O and/or —OH, and in which optionally 1 or 2 carbon atoms, independently of each other, are replaced with an N or S atom,
Z₁ is, in each case independently, ═O, halogen, or an alkyl group containing 1-5 carbon atoms, which is optionally substituted with ═O and/or —OH,
Z₂ is, in each case independently, ═O, or an alkyl group containing 1-5 carbon atoms, which is optionally substituted with ═O and/or —OH,
n is 0, 1, or 2, and
m is 0,or 1; 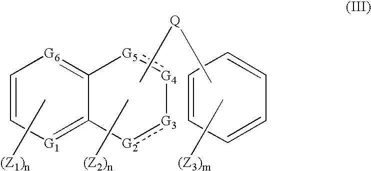
wherein,
G₁, G₂, G₃, G₄, G₅ and G₆ are, in each case independently, C, O, S, or N, such that four or five of G₁, G₂, G₃, G₄, G₅ and G₆ are C atoms and the remaining G₁, G₂, G₃, G₄, G₅ and G₆ are O, S, or N,
Q is a bond or a straight chain or branched alkylene or alkenylene group. containing 1-10 carbon atoms which is optionally substituted with ═O in one or two places, and in which optionally 1 or 2 carbon atoms, independently of each other, are replaced with an N or O atom, and in which optionally a carbon atom is replaced with a 6-membered heterocyclic group containing 1 or 2 nitrogen atoms when the alkylene or alkenylene group is a straight chain group,
Z₁ is, in each case independently, —OH, halogen, or an alkyl group containing 1-5 carbon atoms,
Z₂ is, in each case independently, ═O, halogen, or an alkyl group containing 1-10 carbon atoms which is optionally substituted with ═O in one or two places and/or —OH, and in which optionally 1 or 2 carbon atoms, independently of each other, are replaced with an N and/or S atom,
Z₃ is, in each case independently, —OH, halogen, —NO₂, an alkyl group containing 1-5 carbon atoms, in which optionally a carbon atom is replaced with an O atom, and which is optionally substituted with ═O in one or two places, or is —O-phenyl, wherein the phenyl group in the —O-phenyl is optionally substituted with an —NO₂ group,
n is 0, 1, or 2, and
m is 0, 1, 2, or 3; 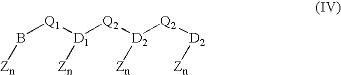
wherein,
B is a phenyl ring,
D₁ is a phenylene ring or a 6-membered saturated or partially or fully unsaturated heterocyclic ring containing 1, 2, or 3 heteroatoms selected from O, S, and N,
D₂ and D₂′ are, each independently of each other, absent or a phenyl or phenylene ring or a 5- or 6-membered saturated or partially or fully unsaturated heterocyclic ring containing 1, 2 or 3 heteroatoms selected from O, S, and N,
Q₁ is a bond or a branched or straight chain allcylene or alkenylenrie group containing 1-10 carbon atoms, which is optionally substituted with 1 to 5 ═O and/or OH groups, in which optionally 1, 2, or 3 carbon atoms are, in each case independently, replaced with an, N, O or S atom, wherein S is optionally substituted with 1 or 2 ═O groups,
Q₂ and Q₂′ are, each independently of each other, a bond or a branched or straight chain alkylene group containing 1-5 carbon atoms, which is optionally substituted with an ═O group, in which optionally a carbon atom is replaced with an N, S, or O atom, wherein Q₂ is absent when D₂ is absent and Q₂′ is absent when D₂′ is absent,
Z is, in each case independently, ═O, ═S, —OH, —NH₂, —NO₂, —C N, —SO₃H, is halogen, or a straight chain or branched alkyl or alkenyl group containing 1 to 10, which is optionally substituted with 1 to 3 ═O and/or OH groups, and in which optionally a carbon atom is replaced with an N, O or S atom, or is a cyclic alkyl group containing 3 carbon atoms,
n is, in each case independently, 0, 1, 2, 3, 4 or 5; 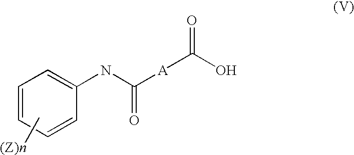
wherein,
Z is, in each case independently, —NO₂, an alkyl containing 1 to 5 carbon atoms, in which optionally a carbon atom is replaced with an O atom, and whidh is optionally substituted with an ═O group,
A is a straight chain alkylene group containing 1 to 5 carbon atoms, and
n is 1, 2 or 3; 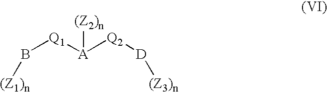
wherein,
B is a phenyl ring,
D is absent, or is a phenyl ring or a 6-membered saturated or partially or fully unsaturated heterocyclic ring containing 1, 2, or 3 heteroatoms selected from O, S, and N,
A is a 5-membered saturated or partially or fully unsaturated heterocyclic ring containing 1, 2, 3 or 4 heteroatoms selected from O, S, and N,
Q₁ and Q₂ are, in each case independently of each other, a bond or a straight chain or branched alkylene group containing 1-5 carbon atoms, in which optionally a carbon atom is replaced with an O, N or S atom, and in which optionally 1 or 2 —C— groups are replaced with —C═0 or ═C— groups, and which is optionally substituted with an ═O group, wherein Q₂ is absent when D is absent,
Z₁ is, in each case independently, —NO₂, —OH, halogen, or an alkyl group containing 1-5 carbon atoms, which is optionally substituted with up to 3 ═O and/or OH groups, and in which optionally a carbon atom is replaced with and N, S or O atom,
Z₂ is, in each case independently, —NH₂, —OH, ═NH, ═O, ═S, phenyl, halogen, or an alkyl group containing 1-5 carbon atoms, which is optionally substituted with up to 3 ═O and/or OH groups, and in which optionally a carbon atom is replaced with an S atom,
Z₃ is, in each case independently, ═O, —OH, NO₂, NH₂, halogen, or an alkyl group containing 1-5 carbon atoms, which is optionally substituted with up to 3═O and/or OH groups, and in which optionally a carbon atom is replaced with an O atom, and
n is, in each case independently, 0, 1, 2 or 3; 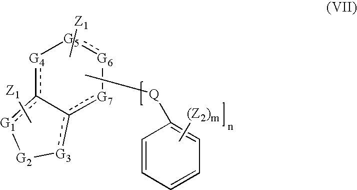
wherein,
G₁ to G₇ are, in each case independently, C, O, S, orN, wherein at least 3 of G₁ to G₇ are C atoms,
Z₁ is, in each case independently, absent, or ═O, ═NH or an alkyl group containing 1 to 5 carbon atoms,
Z₂ is, in each case independently, a straight chain or branched alkyl group containing 1 to 5 carbon atoms, in which optionally a carbon atom is replaced with an O atom, and which is optionally substituted with 1 or 2 ═O and/or —OH groups,
Q is, in each case independently, a bond or an alkylene group containing 1-5 carbon atoms, which is optionally substituted with ═O, in which optionally 1, 2, or 3 carbon atoms are, in each case independently, replaced with an N or S atom, wherein S is optionally substituted with 1 or 2 ═O groups, and
n is 0, 1 or 2, and
m is 1 or 2; 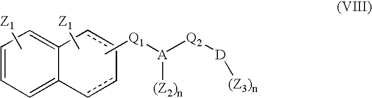
wherein,
A is a 5-membered saturated or partially or fully unsaturated heterocyclic ring containing 1, 2 or 3 heteroatoms selected from O, S, and N, or is a C₁₀ aromatic bi-cyclic ring containing 1, 2 or 3 heteroatoms selected fom O, S, and N,
D is absent or is a fully or partially saturated or unsaturated cyclic ring containing 6 or 7 carbon atoms,
Q₁ and Q₂ are, each independently of each other, a bond or a straight chain or branched alkylene group containing 1-10 carbon atoms, which is optionally substituted with an ═O group, and in which optionally 1, 2 or 3 carbon atoms, independently of each other, are replaced with an N or O atom, and wherein optionally 1-3 carbon atoms are replaced with a —C═and/or ═C—, and/or when the alkylene group is straight chain with a phenyl group, wherein Q₂ is absent when D is absent,
Z₁ is, in each case independently, absent or an alkyl group containing 1 to 5 carbon atoms, in which optionally a carbon atom is replaced with an —O- group, and which is optionally substituted with one or two ═O or —OH groups,
Z₂ is, in each case independently, ═O or an alkyl group containing 1-5 carbon atoms, which is optionally substituted with ═O in one or two places and/or —OH,
Z₃ is halogen, or an alkyl group containing 1 to 5 carbon atoms, which is optionally halogenated, and
n is 1 or 2; 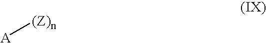
wherein,
A is a 5- or 6-membered saturated or partially or fully unsaturated heterocyclic ring containing 2 or 3 heteroatoms selected from S and N,
Z is, in each case independently, a straight chain or branched alkyl group containing 3-5 carbon atoms, which is substituted with ═O and/or OH groups, and in which a carbon atom is replaced with an S atom, and
n is 1, 2, or 3; 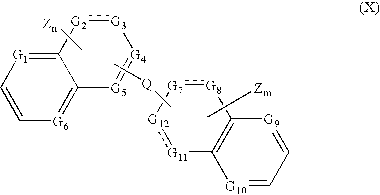
wherein,
G₁ to G₁₂ are, each independently of each other, C, N, S or O,
Z is, in each case independently, an alkyl containing 1 to 5 carbon atoms, which is optionally substituted with 1 to 2 ═O and/or —OH groups,
Q is a bond or an alkylene group containing 1 to 5 carbon atoms,
m 0, 1, 2 or 3,
n 0, 1, 2 or 3, such that
m+n 1; 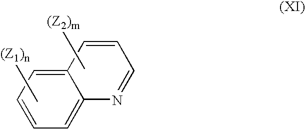
wherein,
Z₁ is, in each case independently, halogen, —NO₂ or —OH,
Z₂ is, in each case independently, an alkyl group containing 1-5 carbon atoms, which is optionally substituted with an ═O and/or —OH group, and in which optionally a carbon atom is replaced with an S atom,
n is 0, 1, 2, or 3,
m is 0, 1, 2, or 3, and
n+m is 3 or more; 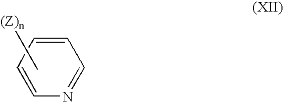
wherein,
Z is, in each case independently, —C N, halogen, or an alkyl group containing 1-5 carbon atoms, which is optionally substituted with halogen, and/or is substituted with one or more ═O and/or —OH groups, and in which optionally a carbon atom is replaced with an S atom, and
n is 2, 3, 4 or 5; 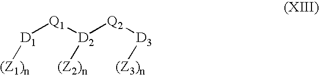
wherein,
D₁ is a 5- or 6-membered saturated or partially or fully unsaturated heterocyclic ring containing 1, 2, or 3 heteroatoms selected from O, S, and N,
D₂ is a 6-membered saturated or partially or fully unsaturated heterocyclic ring containing 1, 2, or 3 heteroatoms selected from O, S, and N, or is optionally a phenylene group when D₃ is present,
D₃ is absent or a 5- or 6-membered saturated or partially or fully unsaturated heterocyclic ring containing 1, 2, or 3 heteroatoms selected from O, S, and N,
Q₁ is —O—, or a straight chain alkylene group containing 1-5 carbon atoms, in which optionally a carbon atom is replaced with an N, O or S atom, and which is optionally substituted with an ═O atom,
Q₂ is absent when D₃ is absent or is a bond or an —O— group,
Z₁ is, in each case independently, ═O or halogen,
Z₂ is, in each case independently, ═O, —C N, —COOH, —NO₂ or halogen,
Z₃ is, in each case independently, halogen, and is absent when D₃ is absent, and
n is, in each case independently, 0, 1, 2, or 3; 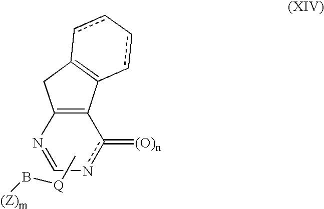
wherein,
B is a phenylene group,
Q is a straight chain allcylene group containing 1-10 carbon atoms, in which optionally up to three carbon atoms are replaced with an N O or S atom, and which is optionally substituted with 1 or 2 ═O groups,
Z is, in each case independently, halogen, or an alkyl group containing 1-5 carbon atoms, in which optionally a carbon atom is replaced with an O atom,
n is 0 or 1, and
m is 1 or 2; 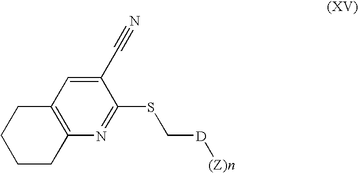
wherein,
D is, a 6-membered saturated or partially or fully unsaturated heterocyclic ring containing 1, 2, or 3 heteroatoms selected from O, S, and N,
Z is ═O
n is 1, or 2; 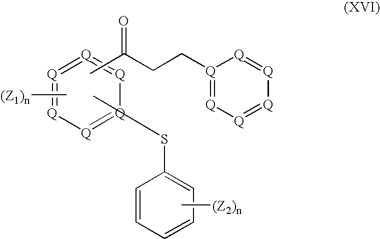
wherein,
Q is, each independently, C or N, wherein,
Z₁ is a phenyl group, or 2 of Z₁ together form with the Q atoms to which they are bound a 6-membered aromatic ring containing only C atoms,
Z₂ is halogen, preferably Cl, and
n isl,or2; 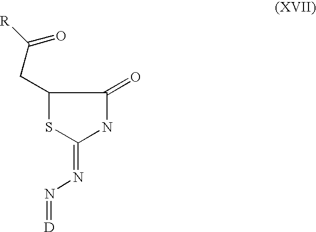
wherein,
D is, a carbocyclic group containing 8 to 10 carbon atoms, and
R is —OH or an alkyl group containing 1 to 5 carbon atoms, in which optionally a carbon atom is replaced with an N or O atom or with a phenyl group, and which is optionally substituted with 1 to 2 ═O and/or —OH groups.

2. A method of modulating the binding of a p56^{lck} molecule via an SH2 domain thereof to a corresponding cellular binding protein, or modulating the activity of a p56^{lck} molecule via an SH2 domain thereof, comprising administering a compound of formula I to XVII or a pharmaceutically acceptable salt thereof 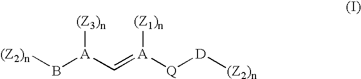
wherein,
B is a phenyl ring,
D is a phenyl ring or a 5-membered saturated or partially or fully unsaturated heterocyclic ring containing 1, 2 or 3 heteroatoms selected from O, S, and N,
A is, in each case independently of each other, a -5-membered saturated or partially or fully unsaturated heterocyclic ring containing 1, 2 or 3 heteroatoms selected from O, S, and N,
Q is a bond or an alkylene or alkenylene group containing 1-5 carbon atoms, which is optionally substituted with ═O, and in which optionally a carbon atom is replaced with. an N atom,
Z₁ is, in each case independently, —NH₂, ═O, ═NH, or ═N-phenyl, -phenyl, or alkyl containing 1 to 5 carbon atoms,
Z₂ is, in each case independently, —OH, halogen, alkyl containing 1-5 carbon atoms, which is optionally substituted with halogen, and/or substituted with ═O and/or —OH, and in which one C atom is optionally replaced with and O atom,
Z₃ is, in each case independently, alkyl containing 1-5 carbon atoms, and
n is, in each case independently, 0, 1, 2, or 3; 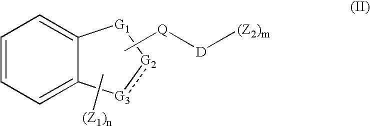
wherein,
G₁, G_{2,} and G₃ are, in each case independently, C, O, S, or N,
D is a phenyl ring or a 5- or 6-membered saturated or partially or fully unsaturated heterocyclic ring containing 1, 2 or 3 heteroatoms selected from O, S, and N,
Q is a straight chain or branched alkylene or alkenylene group containing 1-5 carbon atoms, which is optionally substituted with ═O and/or —OH, and in which optionally 1 or 2 carbon atoms, independently of each other, are replaced with an N or S atom,
Z₁ is, in each case independently, ═O, halogen, or an alkyl group containing 1-5 carbon atoms, which is optionally substituted with ═O and/or —OH,
Z₂ is, in each case independently, ═O, or an alkyl group containing 1-5 carbon atoms, which is optionally substituted with ═O and/or —OH,
n is 0, 1, or 2, and.
m is 0, or 1; 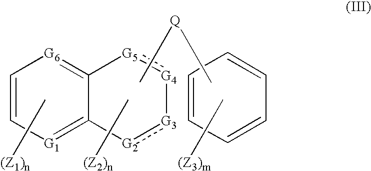
wherein,
G₁, G₂, G₃, G₄, G₅ and G₆ are, in each case independently, C, O, S, or N, such that four or five of G₁, G₂, G₃, G₄, G₅ and G₆ are C atoms and the remaining G₁, G₂, G₃, G₄, G₅ and G₆ are O, S, or N,
Q is a bond or a straight chain or branched alkylene or alkenylene group containing 1 -10 carbon atoms which is optionally substituted with ═O in one or two places, and in which optionally 1 ot 2 carbon atoms, independently of each other, are replaced with an N or O atom, and in which optionally a carbon atom is replaced with a 6-membered heterocyclic group containing 1 or 2 nitrogen atoms when the alkylene or aikenylene group is a straight-chain group,
Z₁ is, in each case independently, —OH, halogen, or an alkyl group containing 1-5 carbon atoms,
Z₂ is, in each case independently, ═O, halogen, or an alkyl group containing 1-10 carbon atoms which is optionally substituted with ═O in one or two places and/or —OH, and in which optionally 1 or 2 carbon atoms, independently of each other, are replaced with an N and/or S atom,
Z₃ is, in each case independently, —OH, halogen, —NO₂, an alkyl group containing 1-5 carbon atoms, in which optionally a carbon atom is replaced with an O atom, and which is optionally substituted with ═O in one or two places, or is —O-phenyl, wherein the phenyl group in the —O-phenyl is optionally substituted with an —NO₂ group,
n is 0, 1, or 2, and
m is 0, 1,2, or 3; 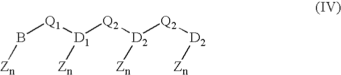
wherein,
B is a phenyl ring,
D₁ is a phenylene ring or a 6-membered saturated or partially or fully unsaturated heterocyclic ring containing 1, 2, or 3 heteroatoms selected from O, S, and N,
D₂ and D₂′ are, each independentlyof each other, absent or a phenyl or phenylene ring or a 5- or 6-membered saturated or partially or fully unsaturated heterocyclic ring containing 1, 2 or 3 heteroatoms selected from O, S, and N,
Q₁ is a bond or a branched or straight chain alkylene or alkenylene group containing 1-10 carbon atoms, which is optionally substituted with 1 to 5 ═O and/or OH groups, in which optionally 1, 2, or 3 carbon atoms are, in each case independently, replaced with an N, O or S atom, wherein S is optionally substityuted with. 1 or 2 ═O groups,
Q₂ and Q₂′ are, each independently of each other, a bond or a branched or straight chain alkylene group containing 1-5 carbon atoms, which is optionally substituted with an ═O group, in which optionally a carbon atom is replaced with an N, S, or O atom, wherein Q₂ is absent when D₂ is absent and Q₂′ is absent when D₂′ is absent,
Z is, in each case independently, ═O, ═S, —OH, —NH_{2,} —NO₂, —C N, —SO₃H, is halogen, or a straight chain or branched allcyl or alkenyl group containing 1 to 10, which is optionally substituted with 1 to 3 ═O and/or OH groups, and in which optionally a carbon atom is replaced with an N, O or S atom, or is a cyclic alkyl group containing 3 carbon atoms,
n is, in each case independently, 0, 1, 2, 3, 4 or 5; 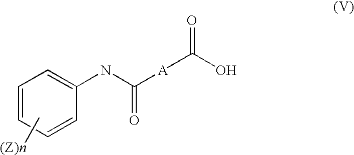
wherein,
Z is, in each case independently, —NO₂, an alkyl containing 1 to 5 carbon atoms, in which optionally a carbon atom is replaced with an O atom, and which is optionally substituted with an ═O group,
A is a straight chain alkylene group containing 1 to 5 carbon atoms, and
n is 1, 2 or 3; 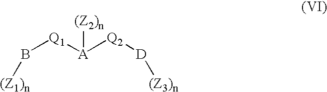
wherein,
B is a phenyl ring,
D is absent, or is a phenyl ring or a 6-membered saturated or partially or fully unsaturated heterocyclic ring containing 1, 2, or 3 heteroatoms selected from O, S, and N,
A is a 5-membered saturated or partially or fully unsaturated heterocyclic ring containing 1, 2, 3 or 4 heteroatoms selected from O, S, and N,
Q₁ and Q₂ are, in each case independently of each other, a bond or a straight chain or branched alkylene group containing 1-5 carbon atoms, in which optionally a carbon atom is replaced with an O, N or S atom, and in which optionally 1 or 2 —C— groups are replaced with —C═ or ═C— groups, and which is optionally substituted with an ═O group, wherein Q₂ is absent when D is absent,
Z₁ is, in each case independently, —NO₂, —OH, halogen, or an alkyl group containing 1-5 carbon atoms, which is optionally substituted with up to 3 ═O and/or OH groups, and in which optionally a carbon atom is replaced with and N, S or O atom,
Z₂ is, in each case independently, —NH₂, —OH, ═NH, ═O, ═S, phenyl, halogen, or an alkyl group containing 1-5 carbon atoms, which is optionally substituted with up to 3 ═O and/or OH groups, and in which optionally a carbon atom is replaced with an S atom,
Z₃ is, in each case independently, ═O, —OH, NO₂, NH₂, halogen, or an alkyl group containing 1-5 carbon atoms, which is optionally substituted with up to 3 ═O and/or OH groups, and in which optionally a carbon atom is replaced with an O atom, and
n is, in each case independently, 0, 1, 2 or 3; 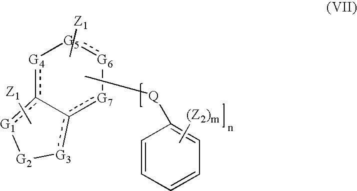
wherein,
G₁ to G₇ are, in each case independently, C, O, S, or N, wherein at least 3 of G₁ to G₇ are C atoms,
Z₁ is, in each case independently, absent, or ═O, ═NH or an alkyl group containing 1 to 5 carbon atoms,
Z₂ is, in each case independently, a straight chain or branched alkyl group containing 1 to 5 carbon atoms, in which optionally a carbon atom is replaced with an O atom, and which is optionally substituted with 1 or 2 ═O and/or —OH groups,
Q is, in each case independently, a bond or an alkylene group containing 1-5 carbon atoms, which is optionally substituted with ═O, in which optionally 1, 2, or 3 carbon atoms are, in each case independently, replaced with an N or S atom, wherein S is optionally substituted with 1 or 2 ═O groups, and
n is 0, or 2, and
m is 1 or 2; 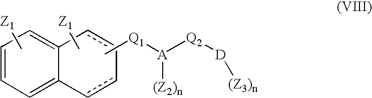
wherein,
A is a 5-membered saturated or partially or fully unsaturated heterocyclic ring containing 1, 2 or 3 heteroatoms selected from O, S, and N, or is a C₁₀ aromatic bi-cyclic ring containing 1, 2 or 3 heteroatoms selected from O, S, and N,
D is absent or is a fully or partially saturated or unsaturated cyclic ring containing 6 or 7 carbon atoms,
Q₁ and Q₂ are, each independently of each other, a bond or a straight chain or branched alkylene group containing 1-10 carbon atoms, which is optionally substituted with an ═O group, and in which optionally 1, 2 or 3 carbon atoms, independently of each other, are replaced with an N or O atom, and wherein optionally 1-3 carbon atoms are replaced with a —C═ and/or ═C—, and/or when the alkylene group is straight chain with a phenyl group, wherein Q₂ is absent when D is absent,
Z₁ is, in each case independently, absent or an alkyl group containing 1 to 5 carbon atoms, in which optionally a carbon atom is replaced with an —O— group, and which is optionally substituted with one or two ═O or —OH groups,
Z₂ is, in each case independently, ═O or an alkyl group containing 1-5 carbon atoms, which is optionally substituted with ═O in one or two places and/or —OH,
Z₃ is halogen, or an alkyl-group containing 1 to 5 carbon atoms, which is optionally halogenated, and
n is 1 or 2; 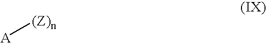
wherein,
A is a 5- or 6-membered saturated or partially or fully unsaturated heterocyclic ring containing 2 or 3 heteroatoms selected from S and N.
Z is, in each case independently, a straight chain or branched alkyl group containing 3-5 carbon atoms, which is substituted with ═O and/or OH groups, and in which a carbon atom is replaced with an S atom, and
n is 1, 2, or 3; 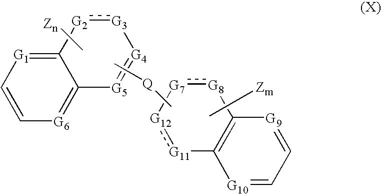
wherein,
G₁ to G₁₂ are, each independently of each other, C, N, S or O,
Z is, in each case independently, an alkyl containing 1 to 5 carbon atoms, which is optionally substituted with 1 to 2 ═O and/or —OH groups,
Q is a bond or an alkylene group containing 1 to 5 carbon atoms,
m 0, 1, 2 or 3,
n 0, 1, 2 or 3, such that
m+n 1; 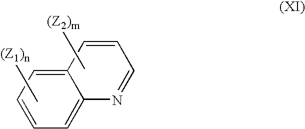
wherein,
Z₁ is, in each case independently, halogen, —NO₂ or —OH,
Z₂ is, in each case independently, an alkyl group containing 1-5 carbon atoms, which is optionally substituted with an ═O and/or —OH group, and in which optionally a carbon atom is replaced with an S atom,
n is 0, 1, 2, or 3,
m is 0, 1, 2, or 3, and
n+m is 3 or more; 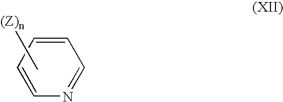
wherein,
Z is, in each case independently, —C N, halogen, or an alkyl group containing 1-5 carbon atoms, which is optionally substituted with halogen, and/or is substituted with one or more ═O and/or —OH groups, and in which optionally a carbon atom is replaced with an S atom, and
n is 2, 3, 4 or 5; 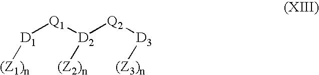
wherein,
D₁ is a 5- or 6-membered saturated or partially or fully unsaturated heterocyclic ring containing 1, 2, or 3 heteroatoms selected from O, S, and N,
D₂ is a 6-membered saturated or partially or fully unsaturated heterocyclic ring-containing 1, 2, or 3 heteroatoms selected from O, S, and N, or is optionally a phenylene group when D₃ is present,
D₃ is absent or a 5- or 6-membered saturated or partially or fully unsaturated heterocyclic ring containing 1, 2, or 3 heteroatoms selected from O, S, and N,
Q₁ is —O—, or a straight chain alkylene group containing 1-5 carbon atoms, in which optionally a carbon atom is replaced with an N, O or S atom, and which is optionally substituted with an ═O atom,
Q₂ is absent when D₃ is absent or is a bond or an —O— group,
Z₁ is, in each case independently, ═O or halogen,
Z₂ is, in each case independently, ═O, —C N, —COOH, —NO₂ or halogen,
Z₃ is, in each case independently, halogen, and is absent when D₃ is absent, and
n is, in each case independently, 0, 1, 2, or 3; 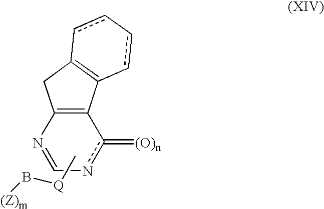
wherein,
B is a phenylene group,
Q is a straight chain alkylene group containing 1-10 carbon atoms, in which optionally up to three carbon atoms are replaced with an N, O or S atom, and which is optionally substituted with 1 or 2 ═O groups,
Z is, in each case independently, halogen, or an alkyl group containing 1-5 carbon atoms, in which optionally a carbon atom is replaced with an O atom,
n is 0 or 1, and
m is 1 or 2; 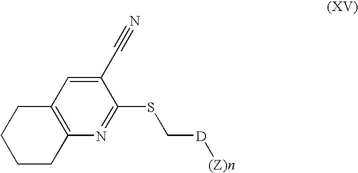
wherein,
D is, a 6-membered saturated or partially or fully unsaturated heterocyclic ring containing 1, 2, or 3 heteroatoms selected from O, S, and N,
z is ═O
n is 1, or 2; 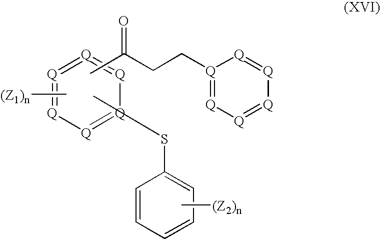
wherein,
Q is, each independently, C or N, wherein,
Z₁ is a phenyl group, or 2 of Z₁ together form with the Q atoms to which they are bound a 6-membered aromatic ring containing only C atoms,
Z₂ is halogen, preferably Cl, and
n is 1, or 2; 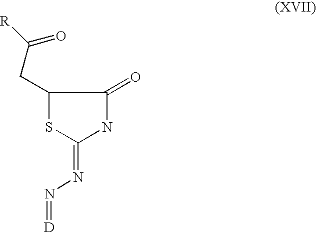
wherein,
D is, a carbocyclic group containing 8 to 10 carbon atoms, and
R is —OH or an alkyl group containing 1 to 5 carbon atoms, in which optionally a carbon atom is replaced with an N or O atom or with a phenyl group, and which is optionally substituted with 1 to 2 ═O and/or —OH groups.

3. A pharmaceutical composition comprising a compound of formula I to XVII or a pharmaceutically acceptable salt thereof 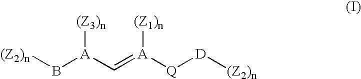
wherein,
B is a phenyl ring,
D is a phenyl ring or a 5-membered saturated or partially or fuilly unsaturated heterocyclic ring containing 1, 2 or 3 heteroatoms selected from O, S, and N,
A is, in each case independently of each other, a 5-membered saturated or partially or fully unsaturated heterocyclic ring containing 1, 2 or 3 heteroatoms selected from O, S, and N,
Q is a bond or an alkylene or alkenylene group containing 1-5 carbon atoms, which is optionally substituted with ═O, and in which optionally a carbon atom is replaced with an N atom,
Z₁ is, in each case independently, —NH₂, ═O, ═NH, or ═N-phenyl, -phenyl, or alkyl containing 1 to 5 carbon atoms,
Z₂ is, in each case independently, —OH, halogen, alkyl containing 1-5 carbon atoms, which is optionally substituted with halogen, and/or substituted with ═O and/or —OH, and in which one C atom is optionally replaced with and O atom,
Z₃ is, in each case independently, alkyl containing 1-5 carbon atoms, and
n is, in each case independently, 0, 1, 2, or 3; 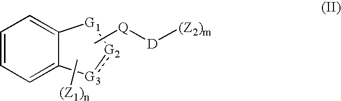
wherein,
G₁, G₂, and G₃ are, in each case independently, C, O, S, or N,
D is a phenyl ring or a 5- or 6-membered saturated or partially or fully unsaturated heterocyclic ring containing 1, 2 or 3 heteroatoms selected from O, S, and N,
Q is a straight chain or branched alkylene or alkenylene group containing 1-5 carbon atoms, which is optionally substituted with ═O and/or —OH, and in which optionally 1 or 2 carbon atoms, independently of each other, are replaced with an N or S atom,
Z₁ is, in each case independently, ═O, halogen, or an alkyl group containing 1-5 carbon atoms, which is optionally substituted with ═O and/or —OH,
Z₂ is, in each case independently, ═O, or an alkyl group containing 1-5 carbon atoms, which is optionally substituted with ═O and/or —OH,
n is 0, 1, or 2, and
m is 0, or 1; 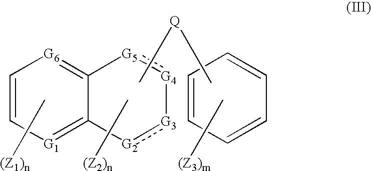
wherein,
G₁, G₂, G₃, G₄, G₅ and G₆ are, in each case independently, C, O, S, or N, such that four or five of G₁, G₂, G₃, G₄, G₅ and G₆ are C atoms and the remaining G₁, G₂, G₃, G₄, G₅ and G₆ are O, S, or N,
Q is a bond or a straight chain or branched alkylene or alkenylene group containing 1-10 carbon atoms which is optionally substituted with ═O in one or two places, and in which optionally 1 or 2 carbon atoms, independently of each other, are replaced with an N or O atom, and in which optionally a carbon atom is replaced with a 6-membered heterocyclic group containing 1 or 2 nitrogen atoms when the alkylene or alkenylene group is a straight chain group,
Z₁ is, in each case independently, —OH, halogen, or an alkyl group containing 1-5 carbon atoms,
Z₂ is, in each case independently, ═O, halogen, or an alkyl group containing 1-10 carbon atoms which is optionally substituted with ═O in one or two places and/or —OH, and in which optionally 1 or 2 carbon atoms, independently of each other, are replaced with an N and/or S atom,
Z₃ is, in each case independently, —OH, halogen, —NO₂, an alkyl group containing 1-5 carbon atoms, in which optionally a carbon atom is replaced with an O atom, and which is optionally substituted with ═O in one or two places, or is —O-phenyl, wherein the phenyl group in the —O-phenyl is optionally substituted with an —NO₂ group,
n is 0, 1, or 2, and
m is 0, 1, 2, or 3; 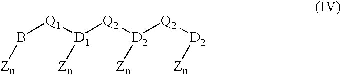
wherein,
B is a phenyl ring,
D₁ is a phenylene ring or a 6-membered saturated or partially or fully unsaturated heterocyclic ring containing 1, 2, or 3 heteroatoms selected from O, S, and N,
D₂ and D₂′ are, each independently of each other, absent or a phenyl or phenylene ring or a 5- or 6-membered saturated or partially or fully unsaturated heterocyclic ring containing 1, 2 or 3 heteroatoms selected from O, S, and N,
Q₁ is a bond or a branched or straight chain alkylene or alkenylene group containing 1-10 carbon atoms, which is optionally substituted with 1 to 5 ═O and/or OH groups, in which optionally 1, 2, or 3 carbon atoms are, in each case independently, replaced with an N, O or S atom, wherein S is optionally substituted with 1 or 2 ═O groups,
Q₂ and Q₂′ are, each independently of each other, a bond or a branched or straight chain alkylene group containing 1-5 carbon atoms, which is optionally substituted with an ═O group, in which optionally a carbon atom is replaced with an N, S, or O atom, wherein Q₂ is absent when D₂ is absent and Q₂′ is absent when D₂′ is absent,
Z is, in each case independently, ═O, ═S, —OH, —NH₂, —NO₂, —C N, —SO₃H, is halogen, or a straight chain or branched alkyl or alkenyl group containing 1 to 10, which is optionally substituted with 1 to 3 ═O and/or OH groups, and in which optionally a carbon atom is replaced with an N, O or S atom, or is a cyclic alkyl group containing 3 carbon atoms,
n is, in each case independently, 0, 1, 2, 3, 4 or 5; 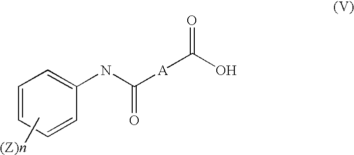
wherein,
z is, in each case independently, —NO₂, an alkyl containing 1 to 5 carbon atoms, in which optionally a carbon atom is replaced with an O atom, and which is optionally substituted with an ═O group,
A is a straight chain alkylene group containing 1 to 5 carbon atoms, and
n is 1, 2 or 3; 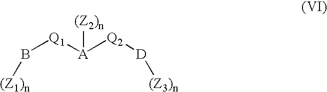
wherein,
B is a phenyl ring,
D is absent, or is a phenyl ring or a 6-membered saturated or partially or fully unsaturated heterocyclic ring containing 1, 2, or 3 heteroatoms selected from O, S, and N,
A is a 5-membered saturated or partially or fully unsaturated heterocyclic ring containing 1, 2, 3 or 4 heteroatoms selected from O, S, and N,
Q₁ and Q₂ are, in each case independently of each other, a bond or a straight chain or branched alkylene group containing 1-5 carbon atoms, in which optionally a carbon atom is replaced with an O, N or S atom, and in which optionally 1 or 2 —C— groups are replaced with —C═ or ═C— groups, and which is optionally substituted with an ═O group, wherein Q₂ is absent when D is absent,
Z₁ is, in each case independently, —NO₂, —OH, halogen, or an alkyl group containing 1-5 carbon atoms, which is optionally substituted with up to 3 ═O and/or OH groups; and in which optionally a carbon atom is replaced with and N, S or O atom,
Z₂ is, in each case independently, —NH₂, —OH, ═NH, ═O, =S, phenyl, halogen, or an alkyl group containing 1-5 carbon atoms, which is optionally substituted with up to 3 ═O and/or OH groups, and in which optionally a carbon atom is replaced with an S atom,
Z₃ is, in each case independently, ═O, —OH, NO₂, NH₂, halogen, or an alkyl group containing 1-5 carbon atoms, which is optionally substituted with up to 3 ═O and/or OH groups, and in which optionally a carbon atom is replaced with an O atom, and
n is, in each case independently, 0, 1, 2 or 3; 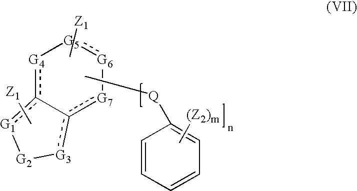
wherein,
G₁ to G₇ are, in each case independently, C, O, S, or N, wherein at least 3 of G₁ to G₇ are C atoms,
Z₁ is, in each case independently, absent, or ═O, ═NH or an alkyl group containing 1 to 5 carbon atoms,
Z₂ is, in each case independently, a straight chain or branched alkyl group containing 1 to 5 carbon atoms, in which optionally a carbon atom is replaced with an O atom, and which is optionally substituted with 1 or 2 ═O and/or —OH groups,
Q is, in each case independently, a bond or an alkylene group containing 1-5 carbon atoms, which is optionally substituted with ═O, in which optionally 1, 2, or 3 carbon atoms are, in each case independently, replaced with an N or S atom, wherein S is optionally substituted with 1 or 2 ═O groups, and
n is 0, 1 or 2, and
m is 1 or 2; 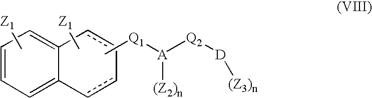
wherein,
A is a 5-membered saturated or partially or fully unsaturated heterocyclic ring containing 1, 2 or 3 heteroatoms selected from O, S, and N, or is a C₁₀ aromatic bi-cyclic ring containing 1, 2 or 3 heteroatoms selected from O, S, and N,
D is absent or is a fully or partially saturated or unsaturated cyclic ring containing 6 or 7 carbon atoms,
Q₁ and Q₂ are, each independently of each other, a bond or a straight chain or branched alkylene group containing 1-10 carbon atoms, which is optionally substituted with an ═O group, and in which optionally 1, 2 or 3 carbon atoms, independently of each other, are replaced with an N or O atom, and wherein optionally 1-3 carbon atoms are replaced with a —C═and/or ═C—, and/or when the alkylene group is straight chain with a phenyl group, wherein Q₂ is absent when D is absent,
Z₁ is, in each case independently, absent or an alkyl group containing 1 to 5 carbon atoms, in which optionally a carbon atom is replaced with an —O— group, and which is optionally substituted with one or two ═O or —OH groups,
Z₂ is, in each case independently, ═O or an alkyl group containing 1-5 carbon atoms, which is optionally substituted with ═O in one or two places and/or —OH,
Z₃ is halogen, or an alkyl group containing 1 to 5 carbon atoms, which is optionally halogenated, and
n is 1 or 2; 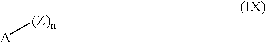
wherein,
A is a 5- or 6-membered saturated or partially or fully unsaturated heterocyclic ring containing 2 or 3 heteroatoms selected from S and N,
Z is, in each case independently, a straight chain or branched alkyl group containing 3-5 carbon atoms, which is substituted with ═O and/or OH groups, and in which a carbon atom is replaced with an S atom, and
n is 1, 2, or 3; 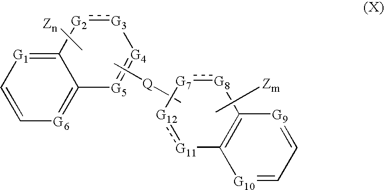
wherein,
G₁ to G₁₂ are, each independently of each other, C, N, S or O,
Z is, in each case independently, an alkyl containing 1 to 5 carbon atoms, which is optionally substituted with 1 to 2 ═O and/or —OH groups,
Q is a bond or an alkylene group containing 1 to 5 carbon atoms,
m 0, 1, 2 or 3,
n 0, 1, 2 or 3, such that
m+n 1; 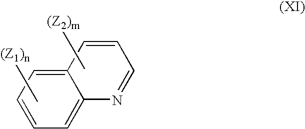
wherein,
Z₁ is, in each case independently, halogen, —NO₂ or —OH,
Z₂ is, in each case independently, an alkyl group containing 1-5 carbon atoms, which is optionally substituted with an ═O and/or —OH group, and in which optionally a carbon atom is replaced with an S atom,
n is 0, 1, 2, or 3,
m is 0, 1, 2, or 3, and
n +m is 3 or more; 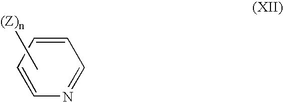
wherein,
Z is, in each case independently, —C N, halogen, or an alkyl group containing 1-5 carbon atoms, which is optionally-substituted with halogen, and/or is substituted with one or more ═O and/or —OH groups, and in which optionally a carbon atom is replaced with an S atom, and
n is 2, 3, 4 or 5; 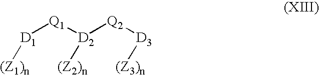
wherein,
D₁ is a 5- or 6-membered saturated or partially or fully unsaturated heterocyclic ring containing 1, 2, or 3 heteroatoms selected from O, S, and N,
D₂ is a 6-membered saturated or partially or fully unsaturated heterocyclic ring containing 1, 2, or 3 heteroatoms selected from O, S, and N, or is optionally a phenylene group when D₃ is present,
D₃ is absent or a 5- or 6-membered saturated or partially or fully unsaturated heterocyclic ring containing 1, 2, or 3 heteroatoms selected from O, S, and N,
Q₁ is —O—, or a straight chain alkylene group containing 1-5 carbon atoms, in which optionally a carbon atom is replaced with an N, O or S atom, and which is optionally substituted with an ═O atom,
Q₂ is absent when D₃ is absent or is a bond or an —O— group,
Z₁ is, in each case independently, ═O or halogen,
Z₂ is, in each case independently, ═O, —C N, —COOH, —NO₂ or halogen,
Z₃ is, in each case independently, halogen, and is absent when D₃ is absent, and
n is, in each case independently, 0, 1, 2, or 3; 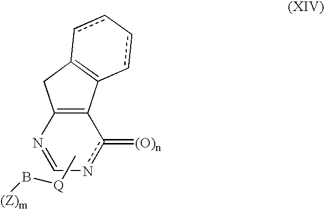
wherein,
B is a phenylene group,
Q is a straight chain alkylene group containing 1-10 carbon atoms, in which optionally up to three carbon atoms are replaced with an N, O or S atom, and which is optionally substituted with 1 or 2 ═O groups,
Z is, in each case independently, halogen, or an alkyl group containing 1-5 carbon atoms, in which optionally a carbon atom is replaced with an O atom,
n is 0 or 1, and
m is 1 or 2; 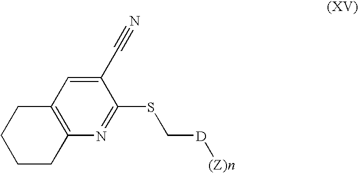
wherein,
D is, a 6-membered saturated or partially or fully unsaturated heterocyclic ring containing 1, 2, or 3 heteroatoms selected from O, S, and N,
Z is ═O
n is 1, or 2; 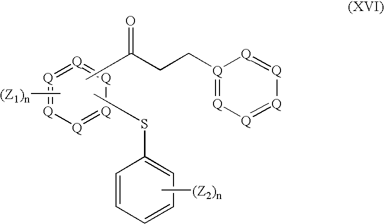
wherein,
Q is, each independently, C or N, wherein,
Z₁ is a phenyl group, or 2 of Z₁ together form with the Q atoms to which they are bound a 6-membered aromatic ring containing only C atoms,
Z₂ is halogen, preferably Cl, and
n is 1, or 2; 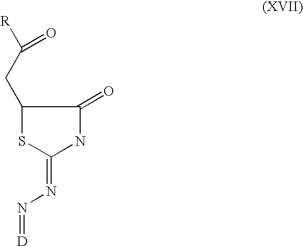
wherein,
D is, a carbocyclic group containing 8 to 10 carbon atoms, and
R is —OH or an alkyl group containing 1 to 5 carbon atoms, in which optionally a carbon atom is replaced with an N or O atom or with a phenyl group, and which is optionally substituted with 1 to 2 ═O and/or —OH groups.

4. A method of claim 1, wherein immunosuppression is affected.

5. A method of claim 1, wherein said patient suffers from an autoimmune disease or from transplant rejection.

6. A method of claim 5, wherein said patient suffers from rheumatoid arthritis.

7. A method of claim 1, wherein said patient suffers from a neoplasm or a hyperplasia.

8. A method of claim 7, wherein said patient suffers from a benign or malignant tumor.

9. A method of claim 1, wherein said patient suffers from a depressed immune system.

10. A method of claim 1, wherein said patient suffers from leukemia, lymphoma, ovarian cancer and breast cancer.

11. A method of claim 1, wherein said patient is human.

12. A method of claim 1, wherein one of the following compounds or a pharmaceutically acceptable salt thereof is administered

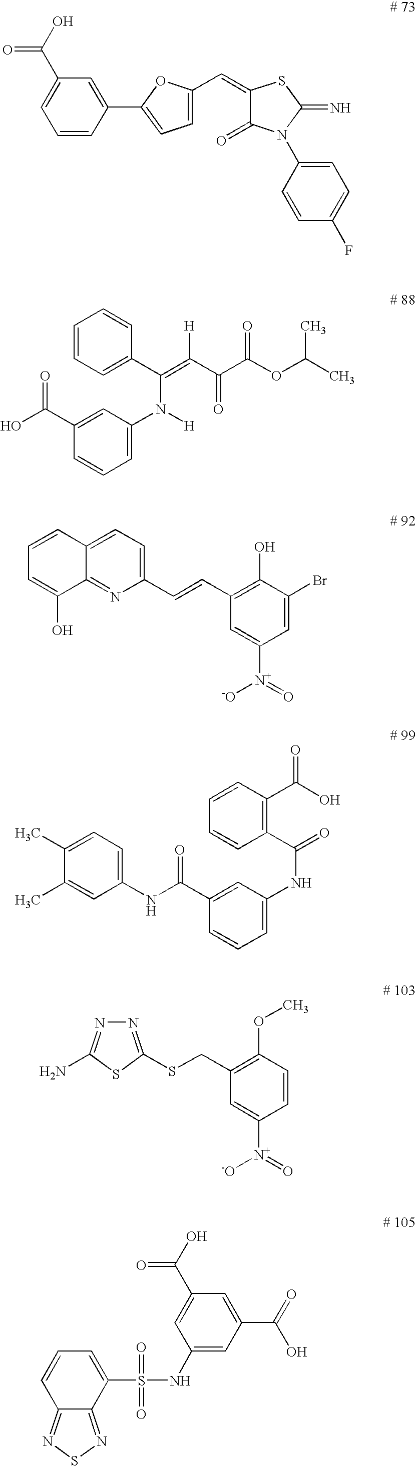

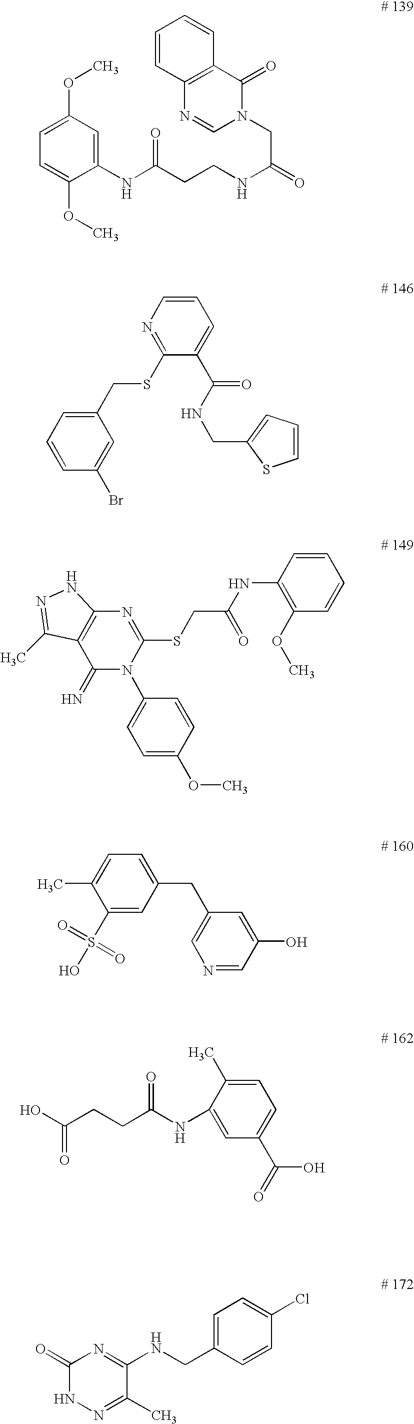

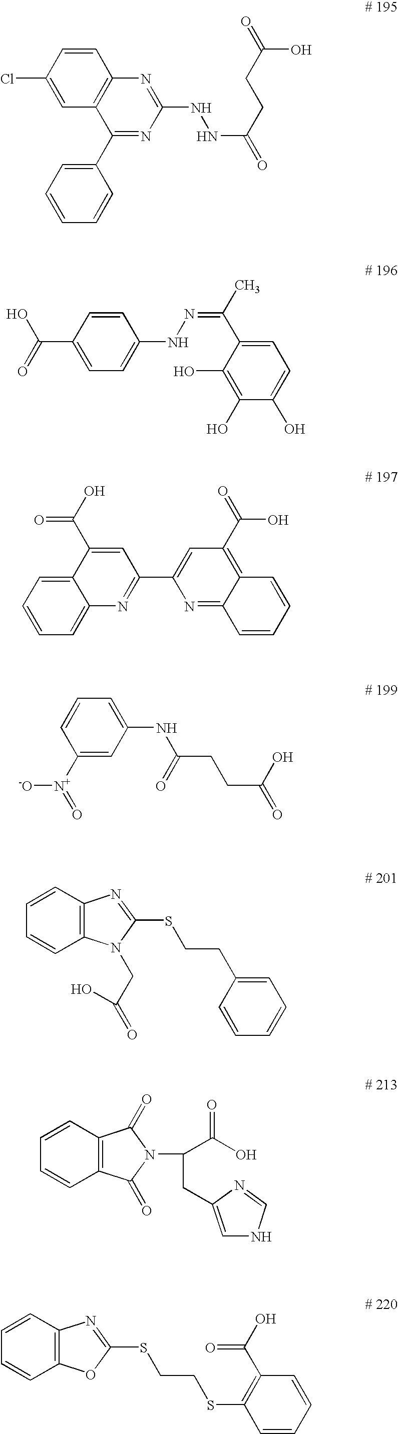

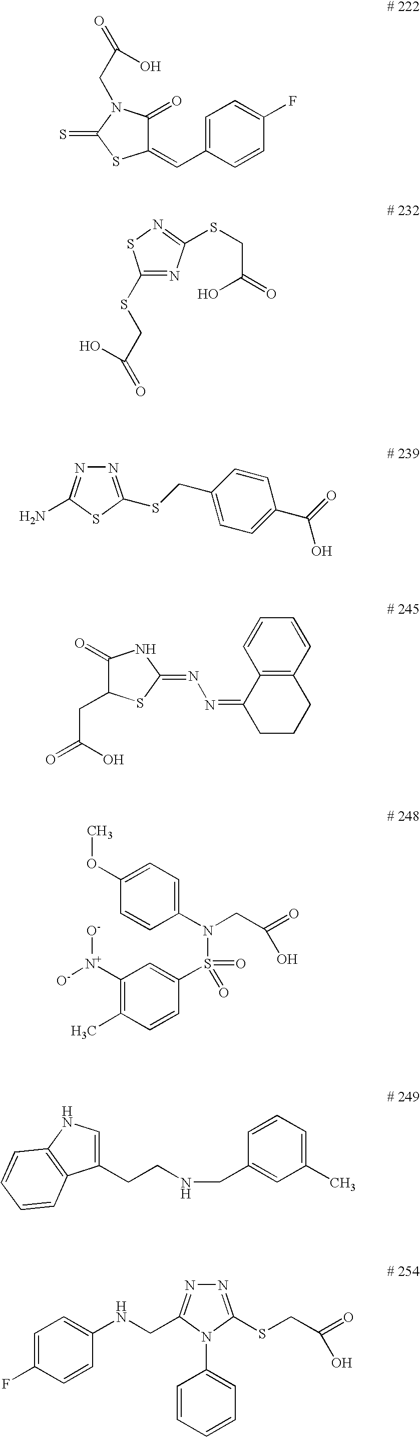

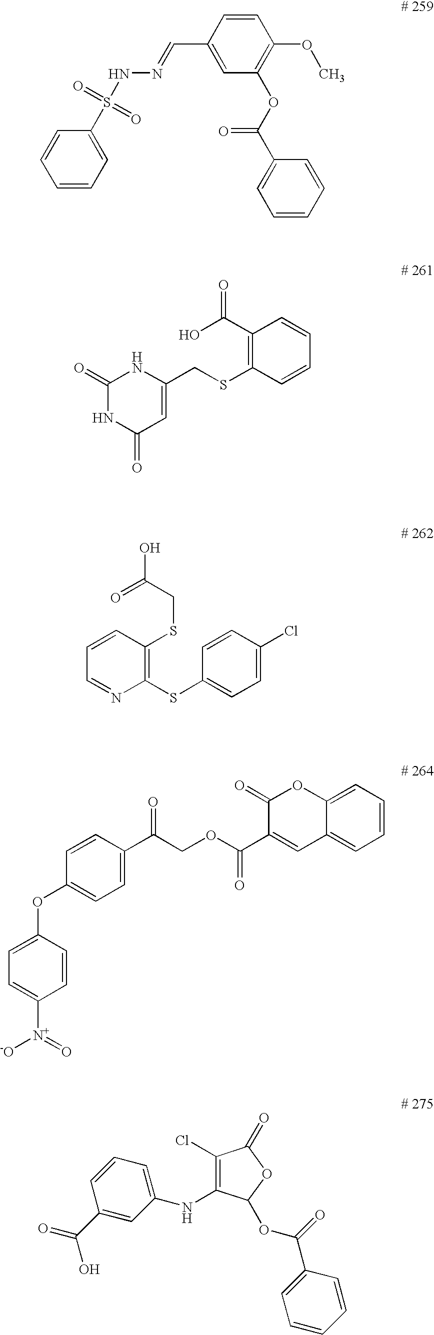

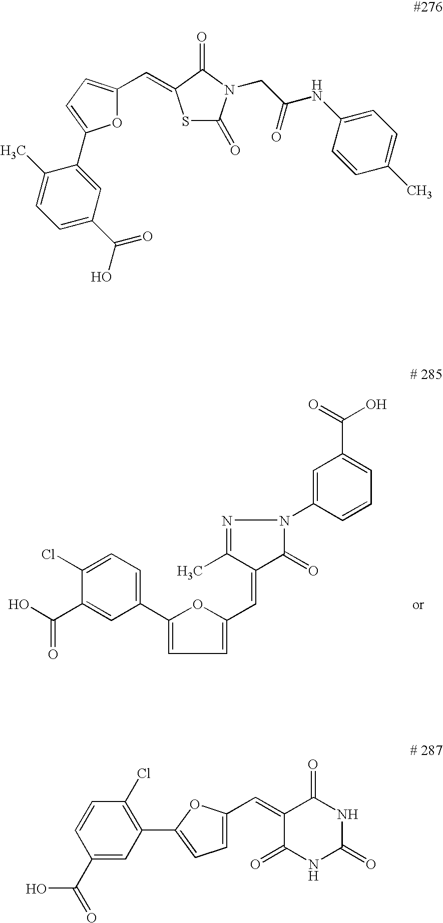

13. A method of claim 2, wherein one of the following compounds or a pharmaceutically acceptable salt thereof is administered

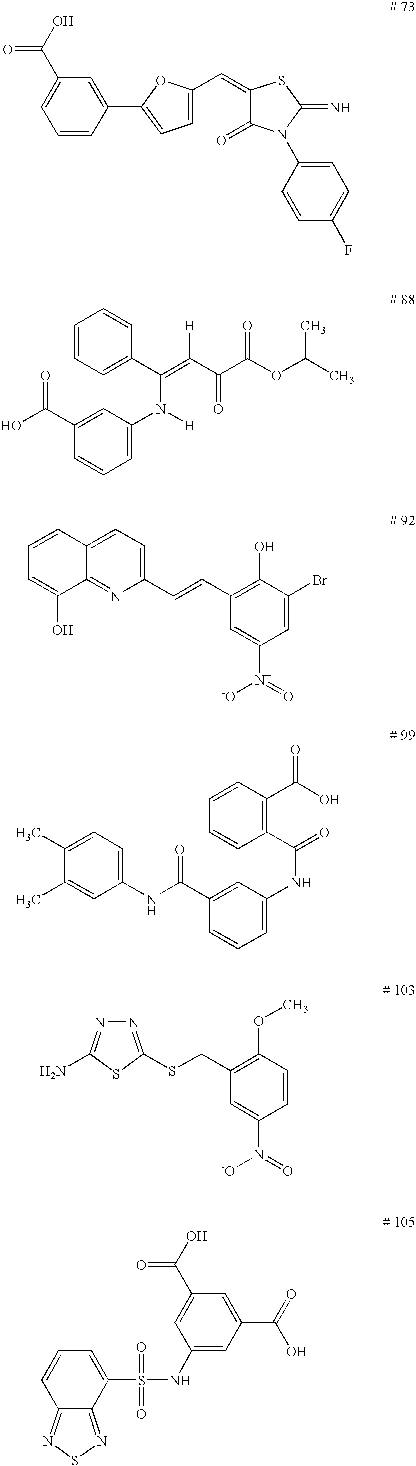

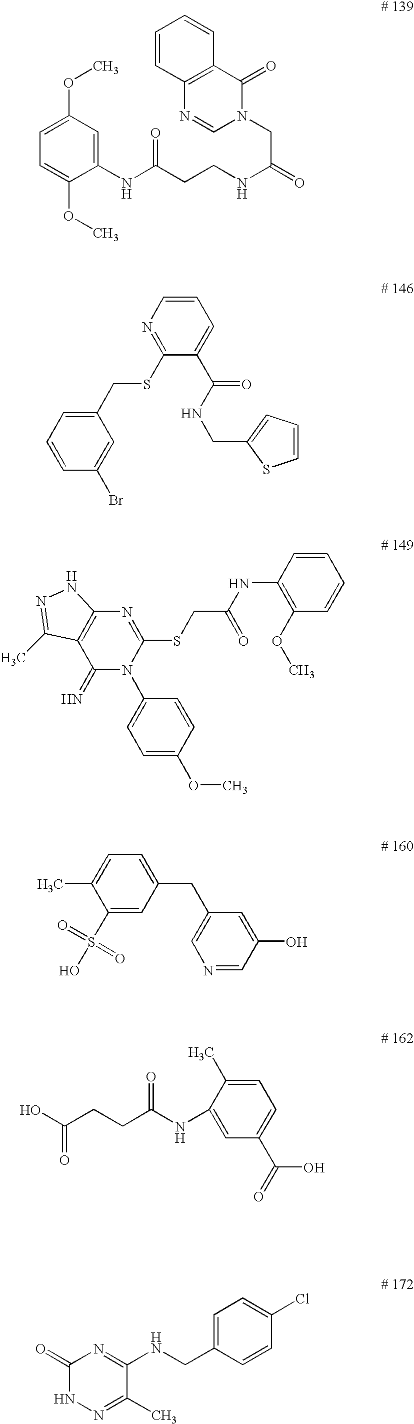

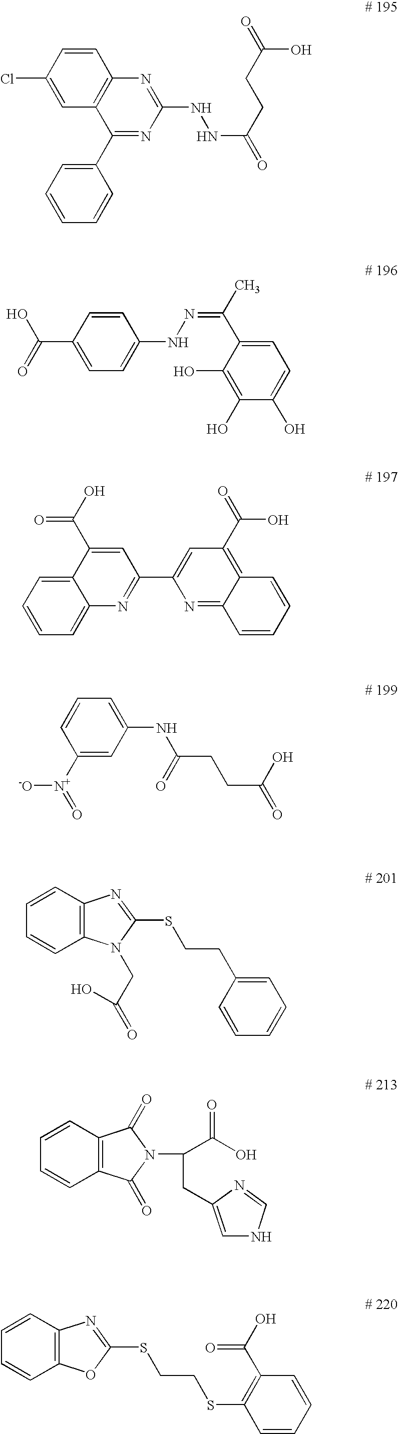

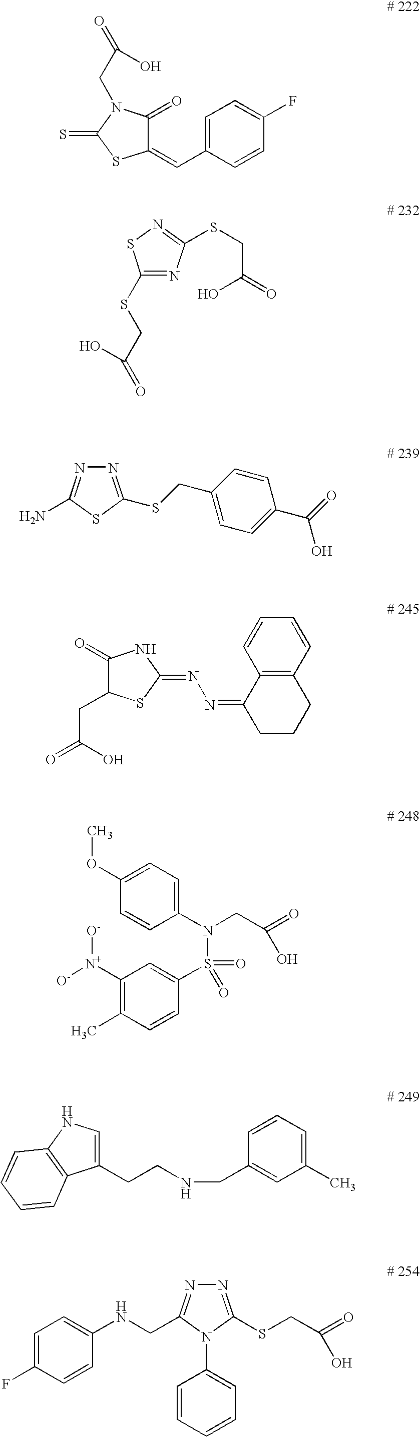

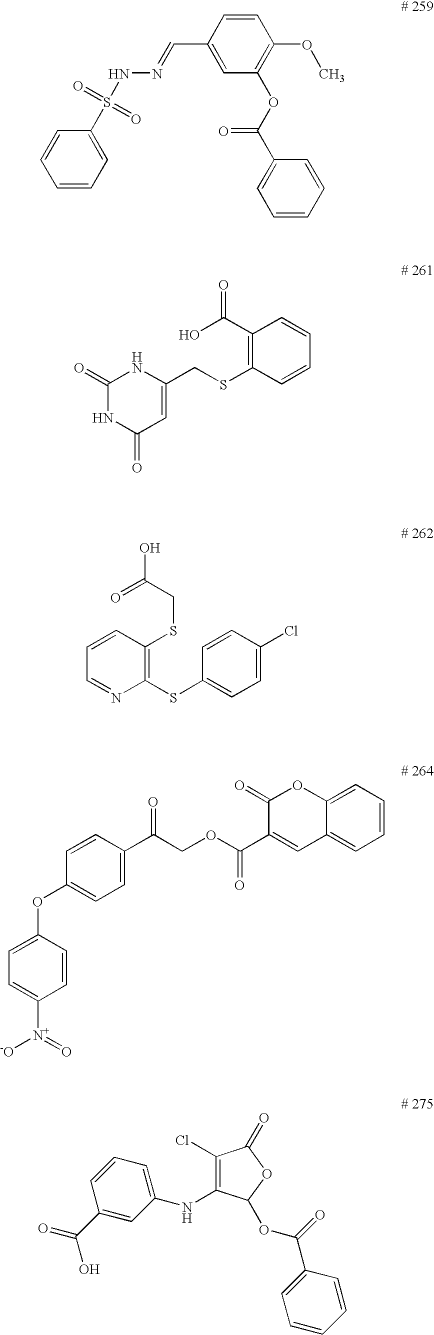

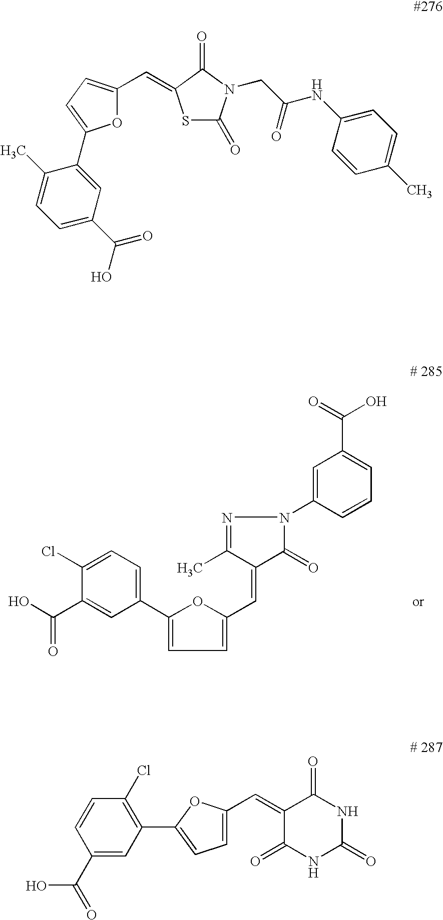

14. A pharmaceutical composition according to claim 3, comprising one of the following compounds or a pharmaceutically acceptable salt thereof

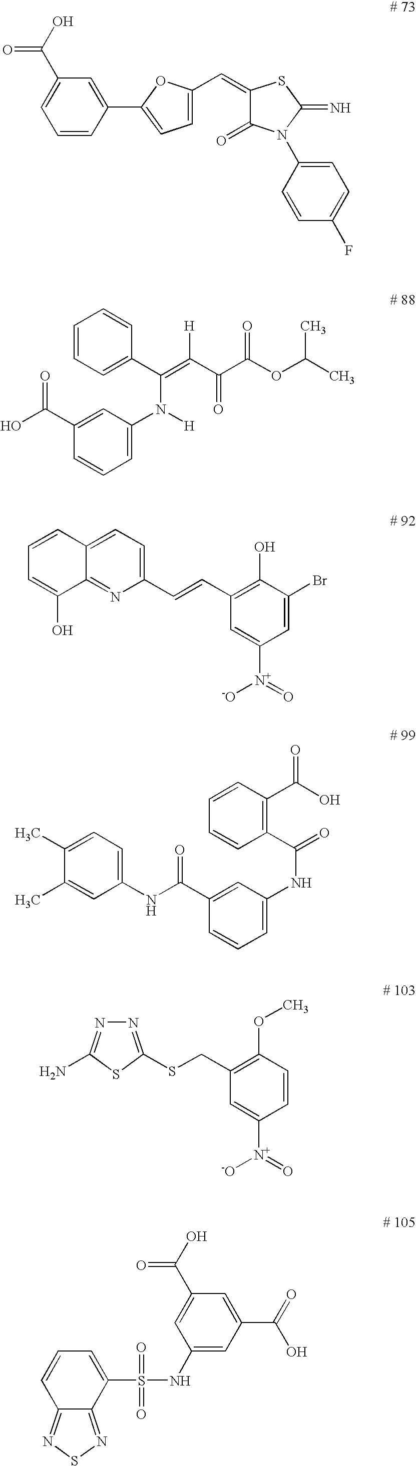

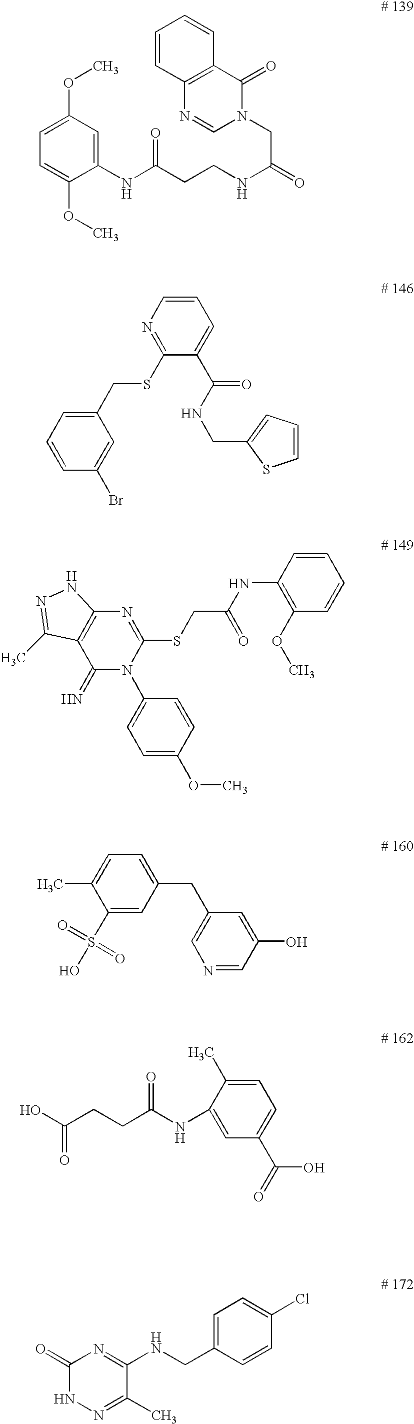

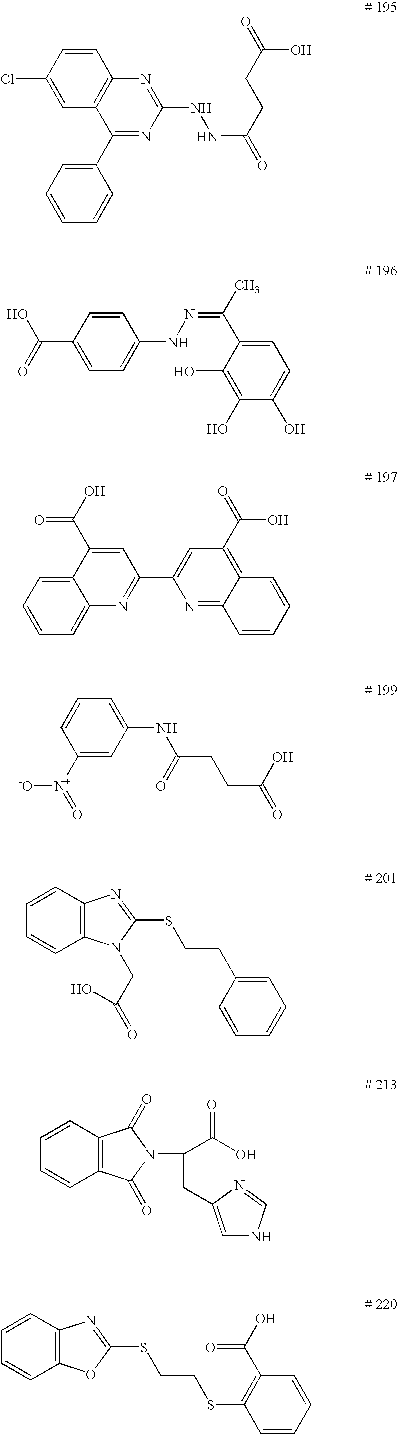

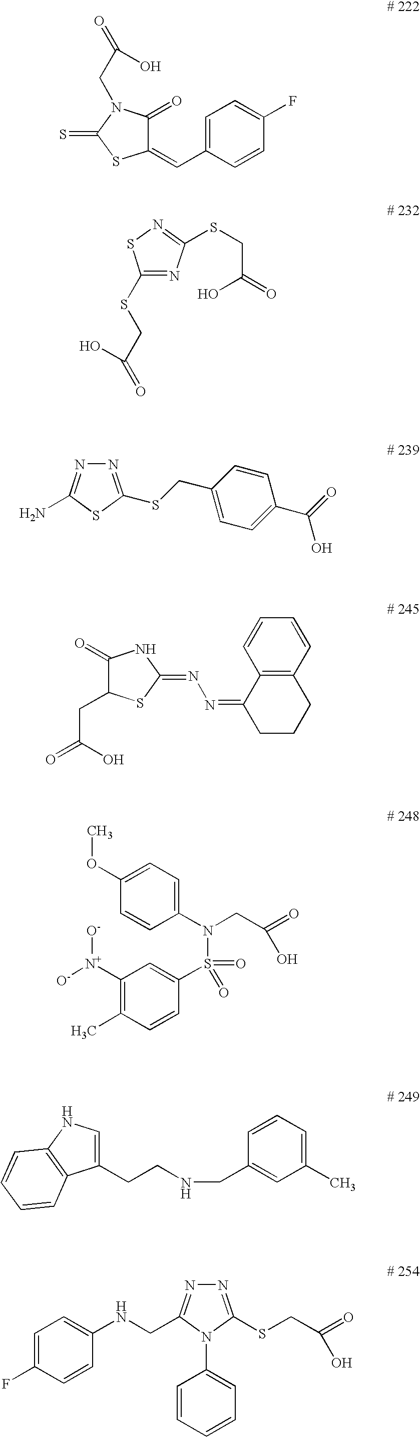

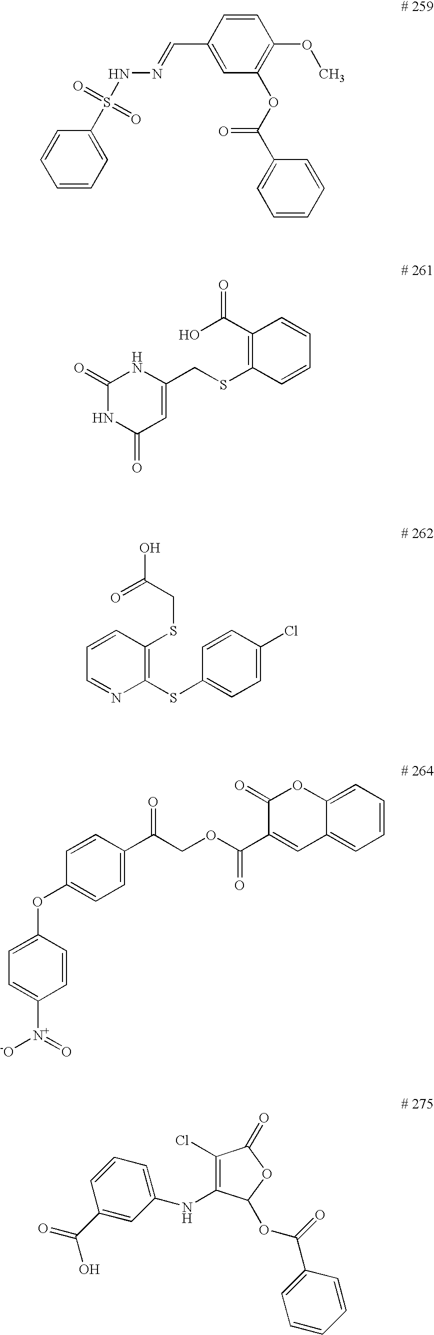

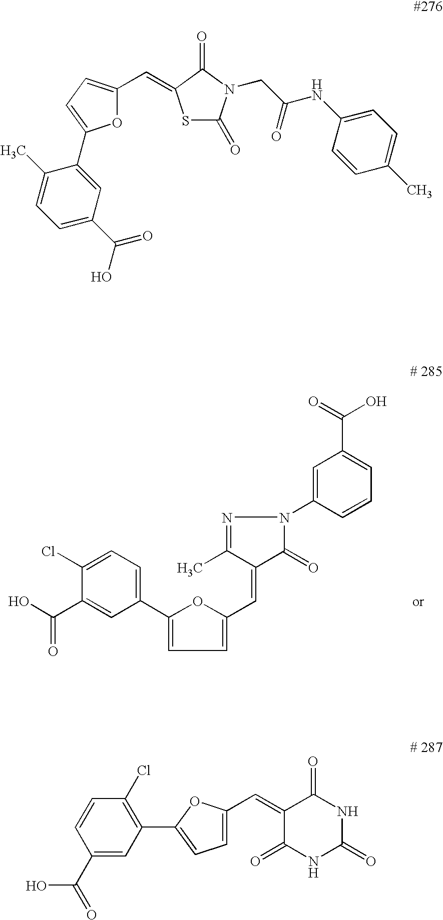

15. A method of claim 12, wherein the compound 73 or 92 ora pharmaceutically acceptable salt thereof is administered.

16. A method of claim 13, wherein the compound 73 or 92 or a pharmaceutically acceptable salt thereof is administered.

17. A pharmaceutical composition according to claim 14, comprising the compound 73 or 92 or a pharmaceutically acceptable salt thereof.

18. A method of claim 1, wherein the compound of formulae I to XVII has a solubility such that the ClogP value is ≦5, a molecular weight of ≦500 Daltons, and ≦10 hydrogen bond donors and acceptors.

19. A method of claim 2, wherein the compound of formulae I to XVII has a solubility such that the ClogP value is ≦5, a molecular weight of ≦500 Daltons, and ≦10 hydrogen bond donors and acceptors.

20. A pharmaceutical composition according to claim 3, wherein the compound of formulae I to XVII has a solubility such that the ClogP value is ≦5, a molecular weight of ≦500 Daltons, and ≦10 hydrogen bond donors and acceptors.

21. A method according to claim 1 comprising administering an effective amount of a compound formulae I to IX or a pharmaceutically acceptable salt thereof.

22. A method according to claim 2 comprising administering a compound of formula I to IX or a pharmaceutically acceptable salt thereof.

23. A pharmaceutical composition according to claim 3 comprising a compound of formula I to IX or a pharmaceutically acceptable salt thereof.
